SEQUENCE LISTING (58 sequences)

SEQ ID NO: 1 - DNA, 1278 bp, Anabaena sp.
  gene (1)..(1278)
atgttccaaaataaaaaacatcggatcgcacttatttctgtttctggagatccagccgttgaaataggtcaagaagaagccggtggtcagaacgtatatgttcgagaagtaggctatgcactagccgaacaaggttggcaagttgatatgttcactcgccgtatcagtcccgaccaggccgagattgtccaacatagtcctaattgccgcactatccgcttacaagcggggccggttgaatttatcggacgtgatcacgtatttgattatttaccggaatttgttgccgaattccaacgcttccaaaagcgccaaggttataactatcaactcattcacacaaattactggttgtcatcttgggtgggaatgcaactgaaaaagcaacaacccttggtgttggtgcatacataccactcattaggagcaatcaaatatcaaacgatcgcagatatacccgccattgcgaatcagcgattagctatagaaaaagcttgtttagagagtgtagacacagtagttgccaccagcccccaagaacagcaacatatgcgcgccctggtttctaagaagggacgcatagagatgattccttgcgggactgacattaataacttcggaaacattgaaaagtcggctgcacgggaaaaactgggaattgagcctgatgccaagatggtattttatgtaggtcgttttgatccccgtaaaggcatagaaaccttagtcagagcggttgctcagtctaggttgagaggtgaagcaaacctccagttagtaattggtggtggtagccgtcctggtcaaagtgatggcagagagcgcgatcgcattgcgaatattgtggctgaactagaactgaacgattgcaccaccttcgctggtcgcctagatcatgaaatcctcccttactactacgctgcggctgatgtttgcgttgtccccagtcactacgaaccctttggtttagttgctattgaagcgatggctagcaaaactcccgtaatcgccagtaatgtaggtggattgcaatttacagtagttccagaagtcacaggtttacttgcacctccacaagatgagtcagcttttgctacagccatagaccgcatattagccaacccaacttggcgagatcagctaggcacagccgcccgccagcgagtggaaaccaccttcagctgggccggtgtagcatcccaattgagtcagctatacactcatctgttaactcaaaatgcgccagaaaagaaggaaaaagaggctgttgcagcgtag

SEQ ID NO: 2 - protein, 425 aa, Anabaena sp.
  PEPTIDE (1)..(425)
MFQNKKHRIALISVSGDPAVEIGQEEAGGQNVYVREVGYALAEQGWQVDMFTRRISPDQAEIVQHSPNCRTIRLQAGPVEFIGRDHVFDYLPEFVAEFQRFQKRQGYNYQLIHTNYWLSSWVGMQLKKQQPLVLVHTYHSLGAIKYQTIADIPAIANQRLAIEKACLESVDTVVATSPQEQQHMRALVSKKGRIEMIPCGTDINNFGNIEKSAAREKLGIEPDAKMVFYVGRFDPRKGIETLVRAVAQSRLRGEANLQLVIGGGSRPGQSDGRERDRIANIVAELELNDCTTFAGRLDHEILPYYYAAADVCVVPSHYEPFGLVAIEAMASKTPVIASNVGGLQFTVVPEVTGLLAPPQDESAFATAIDRILANPTWRDQLGTAARQRVETTFSWAGVASQLSQLYTHLLTQNAPEKKEKEAVAA

SEQ ID NO: 3 - DNA, 1269 bp, Anabaena sp.
  gene (1)..(1269)
atgaactctaacactgaaaaacgcatagctttaatttcagttcacggagacccagcaatcgaaattggcaaagaagaagctggagggcaaaatgtttacgtgcgcgaagtgggtaaagcattagcccaactgggatggcaagtggatatgtttagccgcaaagtgagtcctgaacaagagttaattgttcaccatagcccactttgtcggacaattcggttaacagcagggccagaagaatttgtaccaagagataatggctttaaatatttaccagaatttgtacaacaactgcttcgattccaaaaagaaaacaacgttaattacccattagtgcatacaaactactggctttctagttgggtgggaatgcagttaaaagcaatccaaggaagcaaacaagttcatacttatcactctttaggagcagtcaagtacaaatctatagatacgattcctttggttgctactaaacgtttatcggtagaaaaacaagtattagaaacagcagaaagaatcgttgctaccagtcctcaagaacagcaacatatgcgatcgctagtttctactaaaggttacattgatatcgttccttgcggtacagatattcaccgctttggttcaattgctagacaagccgcaagagcagaattaggaattgatcaagaagcaaaagtggtcttgtatgtaggacgctttgatcaacgtaaaggcatagaaaccttagtacgtgccatgaatgagtctcaattgcgtgacacgaataaactcaaactaattattggtggtggtagtactcctggtaatagcgatggcagagagcgcgatcgcattgaggccattgtgcaagaattgggcatgacggaaatgactagtttcccaggccgcctcagccaagatgtcctccctgcttactacgctgcggctgatgtttgcgttgttcccagtcactatgaaccttttggattggtggcaattgaagcaatggcaagtggtacacctgtagtagccagcgatgttggtggacttcaatttacggtagtttccgagaaaaccggtttattggtaccaccaaaagatattgctgcgttcaacattgcaattgatagaattttgatgaatccacaatggcgggatgagttaggccttgctgcgaggaaacacgttacccacaaatttggttgggaaggagtagctagccaactggatggaatatacactcaattattgacacaacaggttaaagagccagcattggtaactaaatag

SEQ ID NO: 4 - protein, 422 aa, Anabaena sp.
  PEPTIDE (1)..(422)
MNSNTEKRIALISVHGDPAIEIGKEEAGGQNVYVREVGKALAQLGWQVDMFSRKVSPEQELIVHHSPLCRTIRLTAGPEEFVPRDNGFKYLPEFVQQLLRFQKENNVNYPLVHTNYWLSSWVGMQLKAIQGSKQVHTYHSLGAVKYKSIDTIPLVATKRLSVEKQVLETAERIVATSPQEQQHMRSLVSTKGYIDIVPCGTDIHRFGSIARQAARAELGIDQEAKVVLYVGRFDQRKGIETLVRAMNESQLRDTNKLKLIIGGGSTPGNSDGRERDRIEAIVQELGMTEMTSFPGRLSQDVLPAYYAAADVCVVPSHYEPFGLVAIEAMASGTPVVASDVGGLQFTVVSEKTGLLVPPKDIAAFNIAIDRILMNPQWRDELGLAARKHVTHKFGWEGVASQLDGIYTQLLTQQVKEPALVTK

SEQ ID NO: 5 - DNA, 1416 bp, Prochlorococcus marinus
  gene (1)..(1416)
atgattagtttgaaatttttatatttacatttgcatggtttaatacgttctaataatcttgaattaggtagagattcagacactggtggtcaaacgcaatatgttttggaattggtaaaaagcttggctaatacttcagaggttgatcaagttgatatagttacacgtctcattaaagatagtaaaattgatagttcttattcaaaaaagcaagaatttattgcaccgggagcacgaattttaagatttcaatttggacccaataagtatttaagaaaagaattattttggccttatttagatgaattaactcaaaatcttattcagcattatcaaaaatacgaaaataagccaagctttatccatgctcattatgcagatgctggctatgtgggcgttagattaagtcaagctttaaaagtaccttttattttcaccgggcattctttaggaagagagaaaaaaagaaaattactcgaggctggtttaaaaattaatcaaattgaaaagctttactgtataagcgaaagaattaatgcagaagaagagtctttaaaatatgcggatatcgttgtgacaagcactaaacaagaatctgtatctcaatattctcaatatcactctttttcatccgaaaaatcaaaagttattgctcctggagttgatcatactaagtttcatcatattcattcaacaaccgagacctctgaaattgataatatgatgattccttttttgaaagatataagaaagcctcctattttggctatttctagagcagtaagaagaaaaaatattccttctttagtagaagcttatggacgatcagaaaaattaaaaagaaaaactaacttagttttagttttaggttgtagggacaatacatttaaacttgattctcaacaaagagatgttttccaaaagatttttgaaatgatagacaaatataatttatatggaaaagtggcctatcccaaaaaacactctccagcaaatattccttctatatatagatgggcagctagtagtggaggaatttttgtcaatcctgcattaacagaaccttttggattaacattacttgaagcttcttcatgtggtttaccaattattgctacagatgatggaggtcctaatgaaattcatgcaaaatgtgaaaatggcttattagtaaatgtaactgacattaatcagttgaaaattgctcttgaaaaaggtatttcaaatagttctcaatggaagttatggagtagaaatggaatcgaaggagtccatagacattttagttggaatactcatgtcagaaattatctatcaatcttgcaaggccactatgaaaaatcgacaatagtttcatcatcaggaattaaagaaagttgtttgaaaggtagttcctcacttataaaaccccattga

SEQ ID NO: 6 - protein, 471 aa, Prochlorococcus marinus
  PEPTIDE (1)..(471)
MISLKFLYLHLHGLIRSNNLELGRDSDTGGQTQYVLELVKSLANTSEVDQVDIVTRLIKDSKIDSSYSKKQEFIAPGARILRFQFGPNKYLRKELFWPYLDELTQNLIQHYQKYENKPSFIHAHYADAGYVGVRLSQALKVPFIFTGHSLGREKKRKLLEAGLKINQIEKLYCISERINAEEESLKYADIVVTSTKQESVSQYSQYHSFSSEKSKVIAPGVDHTKFHHIHSTTETSEIDNMMIPFLKDIRKPPILAISRAVRRKNIPSLVEAYGRSEKLKRKTNLVLVLGCRDNTFKLDSQQRDVFQKIFEMIDKYNLYGKVAYPKKHSPANIPSIYRWAASSGGIFVNPALTEPFGLTLLEASSCGLPIIATDDGGPNEIHAKCENGLLVNVTDINQLKIALEKGISNSSQWKLWSRNGIEGVHRHFSWNTHVRNYLSILQGHYEKSTIVSSSGIKESCLKGSSSLIKPH

SEQ ID NO: 7 - DNA, 1443 bp, Nostoc punctiforme
  gene (1)..(1443)
atgaatactcttgcttctttaaatttagttaaatctctagaaaattctgcttcagaaacgccaaattctcaacccgtttatgccctaatctcagttcatggcgatccgacagctgaaattggtaaagaaggggcaggtggtcaaaatgtctatgtgcgagaattgggattagcattagcaaaacgtggctgtcaggttgatatgtttacccgacgcgaataccctgaccaagaagaaattgtggaattagcaccaggatgtcgcactattcgtttaaatgctgggccagcaaaattcattactagaaacgatttatttgaatatttaccagaatttgtagaagcctggctgaattttcaacaacgaacagggcgcagctataccttgattcacactaactattggctttctgcttgggtaggattagaacttaaatctcgattgggactaccccaagttcatacctatcactctataggtgcagttaaataccgcaatatggaaaatccgccgcagatttctgcaattcgtaattgtgtggagagggcaattttagaacaagcagattatgtaatatccactagccctcaagaagcggaagatttacgtcagttaatttcgcaacatggtcgtattaaagtcattccctgcgggattaatactgaacactttggttctgtcagtaaagaagttgctcgccaacagttggggattgcttcagattctcagataatcttgtatgtaggacgctttgacccccgcaagggagttgaaaccctggtcagagcttgcgccaatttgccttcagcatttcaactctatctagttggtggttgccgtgaagatggagcagacttcaaagaacaacagcgcattgaaagtttggtgaatgacctgggattggaagccgttacagttttcactggacgaatttctcaagcactgttacctacttactatgccgcaggggatatctgcgttgtaccgagttactatgagccttttggtttagtggcgattgaagcaatggcagccagaacacccgtaattgctagtaatgtgggaggattgcagcatacggtagtgcatggtgaaactggatttttagttcctcctcgtgattctaaagcattggcgatcgctattcacagtttattacaaaacccgactctcaaagagagctatggcaatgctgcacaaaattgggttcagtctcgttttagcactcagggagttgccgcccgagttcacgaactctatcaatctttaacacttgatacatttattcaagaaattattaaaactaaaaagttaactccagatttggaaagacaaatccaaaatttattgaaatcgaaagttttgaaatctaatgaaattaaagctctagaaaaattaattgactctttttctaatgacattgtgcagtgggcaaccagttaa

SEQ ID NO: 8 - protein, 480 aa, Nostoc punctiforme
  PEPTIDE (1)..(480)
MNTLASLNLVKSLENSASETPNSQPVYALISVHGDPTAEIGKEGAGGQNVYVRELGLALAKRGCQVDMFTRREYPDQEEIVELAPGCRTIRLNAGPAKFITRNDLFEYLPEFVEAWLNFQQRTGRSYTLIHTNYWLSAWVGLELKSRLGLPQVHTYHSIGAVKYRNMENPPQISAIRNCVERAILEQADYVISTSPQEAEDLRQLISQHGRIKVIPCGINTEHFGSVSKEVARQQLGIASDSQIILYVGRFDPRKGVETLVRACANLPSAFQLYLVGGCREDGADFKEQQRIESLVNDLGLEAVTVFTGRISQALLPTYYAAGDICVVPSYYEPFGLVAIEAMAARTPVIASNVGGLQHTVVHGETGFLVPPRDSKALAIAIHSLLQNPTLKESYGNAAQNWVQSRFSTQGVAARVHELYQSLTLDTFIQEIIKTKKLTPDLERQIQNLLKSKVLKSNEIKALEKLIDSFSNDIVQWATS

SEQ ID NO: 9 - DNA, 1272 bp, Nostoc punctiforme
  gene (1)..(1272)
atgttccagaataagaaacatcgcattgccctaatttctattgatggcgacccagcggttgaaattggtcaagaagaggctggaggtcaaaatgtttatgtgcgtcaagtaggttatgccttagcccagcaaggttggcaagtggatatgttcactcgtcgtagtaattctgaacaatctgcgattgctcaacatggcccaaactgtcgtactattcggttaaaagctggcccagccgaatttatcgggcgagataacttgttcgaccatttacctgaattcatcgaagaattccagaaatttcagcagcgccaagggtttcattactccttaattcataccaactactggttatcatcttgggtgggtatggaattgaaaaaacagcaatccctgattcaggtacatacttaccattctttaggagccgttaaatacagaagtattggtgatgttcccgtaattgcagcccagcgattagctgtagaaaaagcctgcttggaaactatagactgtgtagttgcaaccagtccacaagaacagaaacacatgcgggtactcgtttctagcaaagggaacattgaaatgattccctgtggcactgatactgacaaatttgggggaattcagcgaactgcggcgcgagaaaagttgggaattgccccagatgccaaaatagttctctatgttggtcgctttgaccgccgcaaaggaattgaaaccttggtaagagctgttgccaagtctagtttaaggggtgaagctaacctccagctagtaattggcggtggtagccgtcccggtcagagtgatgcaatagaacgcgatcgcattgctagcatcgtgactgaactcggattagaaaattgtacaacctttgccggtcgcctagatgaaactgttctccccttctactacgccgccgctgatgtctgcgtagtccccagccattatgaaccttttggtttagttgctattgaggcaatggctagtcagactccagtcgtagctagtgatgttggtgggttgcagtttactgttgtaccagaagtcacagggttacttgcgcctcctaaagatgaagtagcttttgctgctgctatagaccgtattcttattaacccaacttggcgagaccaattaggtgaagcggctcgacaacggacagaaattgcctttagttggtacagtgttggattccgactgactcaactttacactcgtttgttggctcaaactgcatccaatactcgaccccggattgcagcttaa

SEQ ID NO: 10 - protein, 423 aa, Nostoc punctiforme
  PEPTIDE (1)..(423)
MFQNKKHRIALISIDGDPAVEIGQEEAGGQNVYVRQVGYALAQQGWQVDMFTRRSNSEQSAIAQHGPNCRTIRLKAGPAEFIGRDNLFDHLPEFIEEFQKFQQRQGFHYSLIHTNYWLSSWVGMELKKQQSLIQVHTYHSLGAVKYRSIGDVPVIAAQRLAVEKACLETIDCVVATSPQEQKHMRVLVSSKGNIEMIPCGTDTDKFGGIQRTAAREKLGIAPDAKIVLYVGRFDRRKGIETLVRAVAKSSLRGEANLQLVIGGGSRPGQSDAIERDRIASIVTELGLENCTTFAGRLDETVLPFYYAAADVCVVPSHYEPFGLVAIEAMASQTPVVASDVGGLQFTVVPEVTGLLAPPKDEVAFAAAIDRILINPTWRDQLGEAARQRTEIAFSWYSVGFRLTQLYTRLLAQTASNTRPRIAA

SEQ ID NO: 11 - DNA, 1269 bp, Nostoc punctiforme
  gene (1)..(1269)
atgaactctaccaccgaaaaacgtatcgccttgatttccgtccacggagacccggcgattgaaatagggaaagaagaagctgggggacaaaatgtttatgtgcgccaagtgggtgaagcactagcgcagctgggatggcaagttgatatgtttacccgcaaggctagtctggagcaagattcgattgttgaacatagcgacaattgccgaactattcgtttaaaagctgggccccttgagtttgtgccgcgagatgaaatttttgaatatttgccagaatttgtggagaatttcctcaaatttcaggtaaaaaatgagattcaatatgagttagttcacactaattattggctctctagttgggtggggatgcagttaaagaaaatccaagggagtaaacaggttcacacctatcactcattaggagcagtcaaatacaacactatagaaaatattcctctgattgctagtcagcgattggcagtagaaaaacaggtgttagaaacagcagagcgaattgtagcgaccagtccgcaagaacagcaacacatgcgatcgctagtttccactgaaggcaatatcgatattatcccctgtggtacagatattcagcgttttggttccattgggcgagaagcagccagggctgaactggaaattgccaaagatgccaaagttgtattatatgtagggcgttttgaccaacgcaaaggtatagaaaccctagtgcgtgcagtcaacgagtctgaactacgcgactcgaagaatctcaagctaattattggcggtggtagtactccaggtaacagcgacggcatagaacgcgatcgcattgagcaaatcgtccacgaattaggaatcactgacttgaccatcttctctggtcgtctcagtcaagatattttaccaacttattacgctgctgccgatgtctgcgttgttcctagtcactacgaaccatttggactggttgcgatcgaagcgatggcaagcggtacgccggttgtggctagtgatgtcggtggacttcaatttactgtagttaatgaacaaactggtttattagcaccaccacaagatgtaggtgcttttgcgtctgctattgaccgaattctctttaatccagagtggcgagacgaattgggtaaagctggcagaaagcgtactgaaagccaatttagttggcatggtgtcgcaactcagttgagtgaactttacacccaattgttagaaccatcagcaaaagaacctgcattgcttgttaaatag

SEQ ID NO: 12 - protein, 422 aa, Nostoc punctiforme
  PEPTIDE (1)..(422)
MNSTTEKRIALISVHGDPAIEIGKEEAGGQNVYVRQVGEALAQLGWQVDMFTRKASLEQDSIVEHSDNCRTIRLKAGPLEFVPRDEIFEYLPEFVENFLKFQVKNEIQYELVHTNYWLSSWVGMQLKKIQGSKQVHTYHSLGAVKYNTIENIPLIASQRLAVEKQVLETAERIVATSPQEQQHMRSLVSTEGNIDIIPCGTDIQRFGSIGREAARAELEIAKDAKVVLYVGRFDQRKGIETLVRAVNESELRDSKNLKLIIGGGSTPGNSDGIERDRIEQIVHELGITDLTIFSGRLSQDILPTYYAAADVCVVPSHYEPFGLVAIEAMASGTPVVASDVGGLQFTVVNEQTGLLAPPQDVGAFASAIDRILFNPEWRDELGKAGRKRTESQFSWHGVATQLSELYTQLLEPSAKEPALLVK

SEQ ID NO: 13 - DNA, 2133 bp, Synechococcus sp.
  gene (1)..(2133)
atgggaaggggtgtccgtgtccttcatctgcacttgtacggtctgttccgttcccgggatctggagcttggtcgtgatgcggacaccggcggccagaccctctacgtgctggatctcgtgcgcagcctggcccagcgtcccgaggttgatcgggtcgatgtggtgacacgtcttgtgcaggaccgtcgggttgccgcggactatgagcgcccactcgaggtgattgctcccggtgctcgaatcctgcgctttccgtttggtccgaagcgttatctgcgcaaggaacagctttggccgcatctggaagatcttgccgatcagctggtgcatcacctcacccagcccggccacgaggtggattggattcacgcccattacgccgatgccggtttcgtcggggctctggtgagccaacggcttggtttacccctggtattcaccggtcattcccttggccgcgaaaagcaacgtcgtcttctcgccggcggtggcgatcgtcaacagatcgaacaggcctacgccatgagccgtcggattgaagcggaggagcaggcactcacccaggcggatctggtgatcaccagcacgcagcaggaagctgacctgcaatacgcccgctattcgcagtttcgtcgtgatcgcgtccaggtgatcccgcccggcgtggatgccggacgctttcaccccgtttcttccgccgctgaaggggatgctctcgatcagttgctcagcccctttcttcgcgatcccagcaagcctcccttgttggcgatttcccgtgctgtccgccgcaagaacatcccggctctgttggaagccttcggatcctcatcggtgctgcgggaccgccacaatctcgtgttggtgctggggtgccgtgaagatccccgacagatggagaagcagcaacgggatgtgttccagcaggtgttcgatctcgtcgatcgttacgacctctacggctcggtcgcctatccgaaacagcatcgccgctcgcaggtgccggccttctatcgctgggcagttcaacggggaggtctgtttgttaaccctgcgctgacggaaccattcggtctcacgttgctggaggccgcggcctgtggtctgccgatggtcgctaccgatgacggtggtcctcgcgatattcaggcccgctgtgagaacggcctgttggtggatgtaatcgatgccggtgccttgcaggaggcgctggaacgggctggcaaggatgccagtcgctggcggcgctggagtgacaacggcgtggaggcggtgtcgcgacatttcagttgggatgcccatgtctgtcgctatctgggactgatgcaagcccatctgcatcagctgccatcagtcggtccaaggcctcagggttccccggcctcatcgcatcggccggatcatctgttgttgctggatctcgacagcaccctcgattgtcccgatggcccgtcgctcaccgctttgcgcagccagcttgaacgcgatggtcagcgctacggcttggggatcctgaccggtcgatcattggcggcggcgcggcagcgctacggcgatctgcatctgccctcgccgttggtctggatcagccgggcaggcagtgagattcacctgggcgaggatcttcagcccgatcacatctgggcgcagcacatcgataccgattggcagcgtgaatccgtggaggctgtgatggaggatctccacgaccttttagaacttcaaagcgaagagcatcaggggccctggaagctgagttacctgcaacgtcagccggatgaatcggtgttgagccacgtgcgtcagcggctgaggcgggagggtctatcggctcggcctcaacggcgctgccactggtatctggacgtccttcctcggctggcgtcccgcagtgaggcgattcgtcacctagctctgcattggcagctgcccctcgagagggtcatggtgatggccagtcagcagggtgatggtgagttgctccgggggctgccggccacggtggtaccggcagatcatgatccctgcctcgtgcgccatcctcaacagaaacgggtgctgctttcaggtcgccccagccttgcggccgtgctggatggattaagtcattaccggtttcccagtcagcgctga

SEQ ID NO: 14 - protein, 710 aa, Synechococcus sp.
  PEPTIDE (1)..(710)
MGRGVRVLHLHLYGLFRSRDLELGRDADTGGQTLYVLDLVRSLAQRPEVDRVDVVTRLVQDRRVAADYERPLEVIAPGARILRFPFGPKRYLRKEQLWPHLEDLADQLVHHLTQPGHEVDWIHAHYADAGFVGALVSQRLGLPLVFTGHSLGREKQRRLLAGGGDRQQIEQAYAMSRRIEAEEQALTQADLVITSTQQEADLQYARYSQFRRDRVQVIPPGVDAGRFHPVSSAAEGDALDQLLSPFLRDPSKPPLLAISRAVRRKNIPALLEAFGSSSVLRDRHNLVLVLGCREDPRQMEKQQRDVFQQVFDLVDRYDLYGSVAYPKQHRRSQVPAFYRWAVQRGGLFVNPALTEPFGLTLLEAAACGLPMVATDDGGPRDIQARCENGLLVDVIDAGALQEALERAGKDASRWRRWSDNGVEAVSRHFSWDAHVCRYLGLMQAHLHQLPSVGPRPQGSPASSHRPDHLLLLDLDSTLDCPDGPSLTALRSQLERDGQRYGLGILTGRSLAAARQRYGDLHLPSPLVWISRAGSEIHLGEDLQPDHIWAQHIDTDWQRESVEAVMEDLHDLLELQSEEHQGPWKLSYLQRQPDESVLSHVRQRLRREGLSARPQRRCHWYLDVLPRLASRSEAIRHLALHWQLPLERVMVMASQQGDGELLRGLPATVVPADHDPCLVRHPQQKRVLLSGRPSLAAVLDGLSHYRFPSQR

SEQ ID NO: 15 - DNA, 31 bp, artificial sequence
  (1)..(31)
agatctccatggcccaaaataaaaaacatcg

SEQ ID NO: 16 - DNA, 31 bp, artificial sequence
  a fully synthesized primer sequence
gcgaattctcgagctacgctgcaacagcctc

SEQ ID NO: 17 - DNA, 31 bp, artificial sequence
  a fully synthesized primer sequence
agatctccatggcctctaacactgaaaaacg

SEQ ID NO: 18 - DNA, 34 bp, artificial sequence
  a fully synthesized primer sequence
gcgaattctcgagctatttagttaccaatgctgg

SEQ ID NO: 19 - DNA, 30 bp, artificial sequence
  a fully synthesized primer sequence
cgaggaattcgctgcaacagcctctttttc

SEQ ID NO: 20 - DNA, 31 bp, artificial sequence
  a fully synthesized primer sequence
gctcgaattcgctttagttaccaatgctggc

SEQ ID NO: 21 - DNA, 25 bp, artificial sequence
  a fully synthesized primer sequence
gatcacgtatttgattatttaccgg

SEQ ID NO: 22 - DNA, 25 bp, artificial sequence
  a fully synthesized primer sequence
ccggtaaataatcaaatacgtgatc

SEQ ID NO: 23 - DNA, 20 bp, artificial sequence
  a fully synthesized primer sequence
cggaaacattgaaaagtcgg

SEQ ID NO: 24 - DNA, 20 bp, artificial sequence
  a fully synthesized primer sequence
ccgacttttcaatgtttccg

SEQ ID NO: 25 - DNA, 20 bp, artificial sequence
  a fully synthesized primer sequence
gcgatggctagcaaaactcc

SEQ ID NO: 26 - DNA, 20 bp, artificial sequence
  a fully synthesized primer sequence
ggagttttgctagccatcgc

SEQ ID NO: 27 - DNA, 23 bp, artificial sequence
  a fully synthesized primer sequence
gttaattacccattagtgcatac

SEQ ID NO: 28 - DNA, 23 bp, artificial sequence
  a fully synthesized primer sequence
gtatgcactaatgggtaattaac

SEQ ID NO: 29 - DNA, 21 bp, artificial sequence
  a fully synthesized primer sequence
gtggtcttgtatgtaggacgc

SEQ ID NO: 30 - DNA, 21 bp, artificial sequence
  a fully synthesized primer sequence
gcgtcctacatacaagaccac

SEQ ID NO: 31 - DNA, 19 bp, artificial sequence
  a fully synthesized primer sequence
gcaatggcaagtggtacac

SEQ ID NO: 32 - DNA, 19 bp, artificial sequence
  a fully synthesized primer sequence
gtgtaccacttgccattgc

SEQ ID NO: 33 - DNA, 41 bp, artificial sequence
  a fully synthesized primer sequence
agatctccatggctagtttgaaatttttatatttacatttg

SEQ ID NO: 34 - DNA, 33 bp, artificial sequence
  a fully synthesized primer sequence
gcgaattctcgagtcaatggggttttataagtg

SEQ ID NO: 35 - DNA, 32 bp, artificial sequence
  a fully synthesized primer sequence
agagagaattcctgcatggggttttataagtg

SEQ ID NO: 36 - DNA, 34 bp, artificial sequence
  a fully synthesized primer sequence
gtaagatctgccaccatgggaaggggtgtccgtg

SEQ ID NO: 37 - DNA, 36 bp, artificial sequence
  a fully synthesized primer sequence
tagagaattcaagcttcagcgctgactgggaaaccg

SEQ ID NO: 38 - DNA, 30 bp, artificial sequence
  a fully synthesized primer sequence
agagagaattccgcgctgactgggaaaccg

SEQ ID NO: 39 - DNA, 39 bp, artificial sequence
  a fully synthesized primer sequence
gtaagatctgccaccatggctactcttgcttctttaaat

SEQ ID NO: 40 - DNA, 33 bp, artificial sequence
  a fully synthesized primer sequence
tagactcgagaagctttaactggttgcccactg

SEQ ID NO: 41 - DNA, 25 bp, artificial sequence
  a fully synthesized primer sequence
tagactcgagactggttgcccactg

SEQ ID NO: 42 - DNA, 34 bp, artificial sequence
  a fully synthesized primer sequence
gtaagatctgccaccatggtccagaataagaaac

SEQ ID NO: 43 - DNA, 33 bp, artificial sequence
  a fully synthesized primer sequence
tagactcgagaagctttaagctgcaatccgggg

SEQ ID NO: 44 - DNA, 25 bp, artificial sequence
  a fully synthesized primer sequence
tagactcgagagctgcaatccgggg

SEQ ID NO: 45 - DNA, 38 bp, artificial sequence
  a fully synthesized primer sequence
gtaagatctgccaccatggcctctaccaccgaaaaacg

SEQ ID NO: 46 - DNA, 37 bp, artificial sequence
  a fully synthesized primer sequence
tagactcgagaagcttctatttaacaagcaatgcagg

SEQ ID NO: 47 - DNA, 28 bp, artificial sequence
  a fully synthesized primer sequence
tagactcgagtttaacaagcaatgcagg

SEQ ID NO: 48 - DNA, 32 bp, artificial sequence
  a fully synthesized primer sequence
gtaagatatcatatgacaaccacgagcgaaac

SEQ ID NO: 49 - DNA, 36 bp, artificial sequence
  a fully synthesized primer sequence
tagactcgagaagctttcaatcgccgtcattccatg

SEQ ID NO: 50 - DNA, 27 bp, artificial sequence
  a fully synthesized primer sequence
tagactcgagatcgccgtcattccatg

SEQ ID NO: 51 - DNA, 22 bp, artificial sequence
  a fully synthesized primer sequence
gaaattgataatatgatgattc

SEQ ID NO: 52 - DNA, 18 bp, artificial sequence
  a fully synthesized primer sequence
gggataggccacttttcc

SEQ ID NO: 53 - DNA, 3180 bp, Zea mays
  gene (1)..(3180)
atggccgggaacgactggatcaacagctacctggaggctattctggacgctggcggggccgcgggagatctctcggcagccgcaggcagcggggacggccgcgacgggacggccgtggagaagcgggataagtcgtcgctgatgctccgagagcgcggccggttcagccccgcgcgatacttcgtcgaggaggtcatctccggcttcgacgagaccgacctctacaagacctgggtccgcacctcggctatgaggagtccccaggagcggaacacgcggctggagaacatgtcgtggaggatctggaacctcgccaggaagaagaagcagatagaaggagaggaagcctcacgattgtctaaacaacgcatggaatttgagaaagctcgtcaatatgctgctgatttgtctgaagacctatctgaaggagaaaagggagaaacaaataatgaaccatctattcatgatgagagcatgaggacgcggatgccaaggattggttcaactgatgctattgatacatgggcaaaccagcacaaagataaaaagttgtacatagtattgataagcattcatggtcttatacgcggggagaatatggagctgggacgtgattcagatacaggtggtcaggtgaaatatgttgtagaacttgctagggctttaggttcaacaccaggagtatacagagtggatctactaacaaggcagatttctgcacctgatgttgattggagttatggggaacctactgagatgctcagtccaataagttcagaaaactttgggcttgagctgggcgaaagcagtggtgcctatattgtccggataccattcggaccaagagacaaatatatccctaaagagcatctatggcctcacatccaggaatttgttgatggcgcacttgtccatatcatgcagatgtccaaggtccttggagaacaaattggtagtgggcaaccagtatggcctgttgttatacatggacactatgctgatgctggtgattctgctgctttactgtctggggcactcaatgtacccatggtattcactggtcattctcttggcagagataagttggaccagattttgaagcaagggcgtcaaaccagggatgaaataaatgcaacctataagataatgcgtcgaattgaggccgaggaactttgccttgatacatctgaaatcataattacaagtaccaggcaagaaatagaacagcaatggggattatatgatggttttgatctaactatggcccggaaactcagagcaagaataaggcgtggtgtgagctgctttggtcgttacatgccccgtatgattgcaatccctcctggcatggagtttagtcatatagcaccacatgatgttgacctcgacagtgaggaaggaaatggagatggctcaggttcaccagatccacctatttgggctgatataatgcgcttcttctcaaacccccggaagccaatgattcttgctcttgctcgtccggatccgaagaagaatatcactactctagtcaaagcatttggtgaacatcgtgaactgagaaatttagcaaatcttacactgatcatggggaatcgtgatgtcattgatgaaatgtcaagcacaaatgcagctgttttgacttcagcactcaagttaattgataaatatgatctatatggacaagtggcataccccaagcaccataagcaatctgaagttcctgatatttatcgtttagctgcgagaacaaaaggagtttttatcaattgtgcattggttgaaccatttggactcaccttgattgaggctgctgcatatggtctacccatggttgccacccgaaatggtgggcctgtggacatacatcgggttcttgataatggaattcttgttgacccccacaatcaaaatgaaatagctgaggcactttataagcttgtgtcagataagcacttgtggtcacaatgtcgccagaatggtctgaaaaacatccataaattttcatggcctgaacattgccagaactatttggcacgtgtagtcactctcaagcctagacatccccgctggcaaaagaatgatgttgcagctgaaatatctgaagcagattcacccgaggactctctgagggatattcatgacatatcacttaacttaaagctttccttggacagtgaaaaatcaggcagcaaagaagggaattcaaatgctttgagaaggcattttgaggatgcagcgcaaaagttgtcaggtgttaatgacatcaaaaaggatgtgccaggtgagaatggtaagtggtcgtcattgcgtaggaggaagcacatcattgtaattgctgtagactctgtgcaagatgcagactttgttcaggttattaaaaatatttttgaagcttcaagaaatgagagatcaagtggtgctgttggttttgtgttgtcaacggctagagcaatatcagagttacatactttgcttatatctggagggatagaagctagtgactttgatgccttcatatgcaacagtggcagtgatctttgttatccatcttcaagctctgaggacatgcttaaccctgctgagctcccattcatgattgatcttgattatcactcccaaattgaatatcgctggggaggagaaggtttaaggaagacattaattcgttgggcagctgagaaaaacaaagaaagtggacaaaaaatatttattgaggatgaagaatgctcatccacctactgcatttcatttaaagtgtccaatactgcagctgcacctcctgtgaaggagattaggaggacaatgagaatacaagcactgcgttgccatgttttgtacagccatgatggtagcaagttgaatgtaattcctgttttggcttctcgctcacaggctttaaggtatttgtatatccgatggggggtagagctgtcaaacatcaccgtgattgtcggtgagtgtggtgacacagattatgaaggactacttggaggcgtgcacaaaactatcatactcaaaggctcgttcaatactgctccaaaccaagttcatgctaacagaagctattcatcccaagatgttgtatcctttgacaaacaaggaattgcttcaattgagggatatggtccagacaatctaaagtcagctctacggcaatttggtatattgaaagactaa

SEQ ID NO: 54 - protein, 1059 aa, Zea mays
  PEPTIDE (1)..(1059)
MAGNDWINSYLEAILDAGGAAGDLSAAAGSGDGRDGTAVEKRDKSSLMLRERGRFSPARYFVEEVISGFDETDLYKTWVRTSAMRSPQERNTRLENMSWRIWNLARKKKQIEGEEASRLSKQRMEFEKARQYAADLSEDLSEGEKGETNNEPSIHDESMRTRMPRIGSTDAIDTWANQHKDKKLYIVLISIHGLIRGENMELGRDSDTGGQVKYVVELARALGSTPGVYRVDLLTRQISAPDVDWSYGEPTEMLSPISSENFGLELGESSGAYIVRIPFGPRDKYIPKEHLWPHIQEFVDGALVHIMQMSKVLGEQIGSGQPVWPVVIHGHYADAGDSAALLSGALNVPMVFTGHSLGRDKLDQILKQGRQTRDEINATYKIMRRIEAEELCLDTSEIIITSTRQEIEQQWGLYDGFDLTMARKLRARIRRGVSCFGRYMPRMIAIPPGMEFSHIAPHDVDLDSEEGNGDGSGSPDPPIWADIMRFFSNPRKPMILALARPDPKKNITTLVKAFGEHRELRNLANLTLIMGNRDVIDEMSSTNAAVLTSALKLIDKYDLYGQVAYPKHHKQSEVPDIYRLAARTKGVFINCALVEPFGLTLIEAAAYGLPMVATRNGGPVDIHRVLDNGILVDPHNQNEIAEALYKLVSDKHLWSQCRQNGLKNIHKFSWPEHCQNYLARVVTLKPRHPRWQKNDVAAEISEADSPEDSLRDIHDISLNLKLSLDSEKSGSKEGNSNALRRHFEDAAQKLSGVNDIKKDVPGENGKWSSLRRRKHIIVIAVDSVQDADFVQVIKNIFEASRNERSSGAVGFVLSTARAISELHTLLISGGIEASDFDAFICNSGSDLCYPSSSSEDMLNPAELPFMIDLDYHSQIEYRWGGEGLRKTLIRWAAEKNKESGQKIFIEDEECSSTYCISFKVSNTAAAPPVKEIRRTMRIQALRCHVLYSHDGSKLNVIPVLASRSQALRYLYIRWGVELSNITVIVGECGDTDYEGLLGGVHKTIILKGSFNTAPNQVHANRSYSSQDVVSFDKQGIASIEGYGPDNLKSALRQFGILKD

SEQ ID NO: 55 - DNA, 3180 bp, Zea mays
  gene (1)..(3180)
atggccgggaacgactggatcaacagctacctggaggctattctggacgctggctgggccgcgggagatctctcggcagccgcaggcagcggggacggccgcgacgggacggccgtggagaagcgggataagtcgtcgctgatgctccgagagcgcggccggttcagccccgcgcgatacttcgtcgaggaggtcatctccggcttcgacgagaccgacctctacaagacctgggtccgcacctcggctatgaggagtccccaggagcggaacacgcggctggagaacatgtcgtggaggatctggaacctcgccaggaagaagaagcagatagaaggagaggaagcctcacgattgtctaaacaacgcatggaatttgagaaagctcgtcaatatgctgctgatttgtctgaagacctatctgaaggagaaaagggagaaacaaataatgaaccatctattcatgatgagagcatgaggacgcggatgccaaggattggttcaactgatgctattgatacatgggcaaaccagcacaaagataaaaagttgtacatagtattgataagcattcatggtcttatacgcggggagaatatggagctgggacgtgattcagatacaggtggtcaggtgaaatatgttgtagaacttgctagggctttaggttcaacaccaggagtatacagagtggatctactaacaaggcagatttctgcacctgatgttgattggagttatggggaacctactgagatgctcagtccaataagttcagaaaactttgggcttgagctgggcgaaagcagtggtgcctatattgtccggataccattcggaccaagagacaaatatatccctaaagagcatctatggcctcacatccaggaatttgttgatggcgcacttgtccatatcatgcagatgtccaaggtccttggagaacaaattggtagtgggcaaccagtatggcctgttgttatacatggacactatgctgatgctggtgattctgctgctttactgtctggggcactcaatgtaccaatggtattcactggtcattctcttggcagagataagttggaccagattttgaagcaagggcgtcaaaccagggatgaaataaatgcaacctataagataatgcgtcgaattgaggccgaggaactttgccttgatacatctgaaatcataattacaagtaccaggcaagaaatagaacagcaatggggattatatgatggttttgatctaactatggcccggaaactcagagcaagaataaggcgtggtgtgagctgctttggtcgttacatgccccgtatgattgcaatccctcctggcatggagtttagtcatatagcaccacatgatgttgacctcgacagtgaggaaggaaatggagatggctcaggttcaccagatccacctatttgggctgatataatgcgcttcttctcaaacccccggaagccaatgattcttgctcttgctcgtccggacccgaaaaagaatatcactactctagtcaaagcatttggtgaacatcgtgaactgagaaatttagcaaatcttacactgatcatggggaatcgtgatgtcattgatgaaatgtcaagcacaaatgcagctgttttgacttcagcactcaagttaattgataaatatgatctatatggacaagtggcataccccaagcaccataagcaatctgaagttcctgatatttatcgtttagctgcgagaacaaaaggagtttttatcaattgtgcattggttgaaccatttggactcaccttgattgaggctgctgcatatggtctaccaatggttgccacccgaaatggtgggcctgtggacatacatcgggttcttgataatggaatccttgttgacccccacaatcaaaatgaaatagctgaggcactttataagcttgtgtcagataagcacttgtggtcacaatgtcgccagaatggtctgaaaaacatccataaattttcatggcctgaacattgccagaactatttggcacgtgtagtcactctcaagcctagacatccccgctggcaaaagaatgatgttgcagctgaaatatctgaagcagattcacccgaggactctctgagggatattcatgacatatcacttaacttaaagctttccttggacagtgaaaaatcaggcagcaaagaagggaactcaaatgctttgagaaggcattttgaggatgcagcgcaaaagttgtcaggtgttaatgacatcaaaaaggatgtgccaggtgagaatggtaagtggtcgtcattgcgtaggaggaagcacatcattgtaattgctgtagactctgtgcaagatgcagactttgttcaggttattaaaaatatttttgaagcttcaagaaatgagagatcaagtggtgctgttggttttgtgttgtcaacggctagagcaatatcagagttacatactttgcttatatctggagggatagaagctagtgactttgatgccttcatatgcaacagtggcagtgatctttgttatccatcttcaagctctgaggacatgcttaaccctgctgagctcccattcgtgattgatcttgattatcactcccaaattgaatatcgctggggaggagaaggtttaaggaagacattaattcgttgggcagctgagaaaaacaaagaaagtggacaaaaaatatttattgaggatgaagaatgctcatccacctactgcatttcatttaaagtgtccaatactgcagctgcacctcctgtgaaggagattaggaggacaatgagaatacaagcactgcgttgccatgttttgtacagccatgatggtagcaagttgaatgtaattcctgttttggcttctcgctcacaggctttaaggtatttgtatatccgatggggggtagagctgtcaaacatcaccgtgattgtcggtgagtgtggtgacacagattatgaaggactacttggaggcgtgcacaaaactatcatactcaaaggctcgttcaatactgctccaaaccaagttcatgctaacagaagctattcatcccaagatgttgtatcctttgacaaacaaggaattgcttcaattgagggatatggtccagacaatctaaagtcagctctacggcaatttggtatattgaaagactaa

SEQ ID NO: 56 - protein, 1059 aa, Zea mays; identical to SEQ ID NO: 54
  PEPTIDE (1)..(1059)

SEQ ID NO: 57 - DNA, 2694 bp, Zea mays
  gene (1)..(2694)
atgccaaggattggttcaactgatgctattgatacatgggcaaaccagcacaaagataaaaagttgtacatagtattgataagcattcatggtcttatacgcggggagaatatggagctgggacgtgattcagatacaggtggtcaggtgaaatatgttgtagaacttgctagggctttaggttcaacaccaggagtatacagagtggatctactaacaaggcagatttctgcacctgatgttgattggagttatggggaacctactgagatgctcagtccaataagttcagaaaactttgggcttgagctgggcgaaagcagtggtgcctatattgtccggataccattcggaccaagagacaaatatatccctaaagagcatctatggcctcacatccaggaatttgttgatggcgcacttgtccatatcatgcagatgtccaaggtccttggagaacaaattggtagtgggcaaccagtatggcctgttgttatacatggacactatgctgatgctggtgattctgctgctttactgtctggggcactcaatgtaccaatggtattcactggtcattctcttggcagagataagttggaccagattttgaagcaagggcgtcaaaccagggatgaaataaatgcaacctataagataatgcgtcgaattgaggccgaggaactttgccttgatacatctgaaatcataattacaagtaccaggcaagaaatagaacagcaatggggattatatgatggttttgatctaactatggcccggaaactcagagcaagaataaggcgtggtgtgagctgctttggtcgttacatgccccgtatgattgcaatccctcctggcatggagtttagtcatatagcaccacatgatgttgacctcgacagtgaggaaggaaatggagatggctcaggttcaccagatccacctatttgggctgatataatgcgcttcttctcaaacccccggaagccaatgattcttgctcttgctcgtccggacccgaaaaagaatatcactactctagtcaaagcatttggtgaacatcgtgaactgagaaatttagcaaatcttacactgatcatggggaatcgtgatgtcattgatgaaatgtcaagcacaaatgcagctgttttgacttcagcactcaagttaattgataaatatgatctatatggacaagtggcataccccaagcaccataagcaatctgaagttcctgatatttatcgtttagctgcgagaacaaaaggagtttttatcaattgtgcattggttgaaccatttggactcaccttgattgaggctgctgcatatggtctaccaatggttgccacccgaaatggtgggcctgtggacatacatcgggttcttgataatggaatccttgttgacccccacaatcaaaatgaaatagctgaggcactttataagcttgtgtcagataagcacttgtggtcacaatgtcgccagaatggtctgaaaaacatccataaattttcatggcctgaacattgccagaactatttggcacgtgtagtcactctcaagcctagacatccccgctggcaaaagaatgatgttgcagctgaaatatctgaagcagattcacccgaggactctctgagggatattcatgacatatcacttaacttaaagctttccttggacagtgaaaaatcaggcagcaaagaagggaactcaaatgctttgagaaggcattttgaggatgcagcgcaaaagttgtcaggtgttaatgacatcaaaaaggatgtgccaggtgagaatggtaagtggtcgtcattgcgtaggaggaagcacatcattgtaattgctgtagactctgtgcaagatgcagactttgttcaggttattaaaaatatttttgaagcttcaagaaatgagagatcaagtggtgctgttggttttgtgttgtcaacggctagagcaatatcagagttacatactttgcttatatctggagggatagaagctagtgactttgatgccttcatatgcaacagtggcagtgatctttgttatccatcttcaagctctgaggacatgcttaaccctgctgagctcccattcgtgattgatcttgattatcactcccaaattgaatatcgctggggaggagaaggtttaaggaagacattaattcgttgggcagctgagaaaaacaaagaaagtggacaaaaaatatttattgaggatgaagaatgctcatccacctactgcatttcatttaaagtgtccaatactgcagctgcacctcctgtgaaggagattaggaggacaatgagaatacaagcactgcgttgccatgttttgtacagccatgatggtagcaagttgaatgtaattcctgttttggcttctcgctcacaggctttaaggtatttgtatatccgatggggggtagagctgtcaaacatcaccgtgattgtcggtgagtgtggtgacacagattatgaaggactacttggaggcgtgcacaaaactatcatactcaaaggctcgttcaatactgctccaaaccaagttcatgctaacagaagctattcatcccaagatgttgtatcctttgacaaacaaggaattgcttcaattgagggatatggtccagacaatctaaagtcagctctacggcaatttggtatattgaaagactaa

SEQ ID NO: 58 - protein, 897 aa, Zea mays
  PEPTIDE (1)..(897)
MPRIGSTDAIDTWANQHKDKKLYIVLISIHGLIRGENMELGRDSDTGGQVKYVVELARALGSTPGVYRVDLLTRQISAPDVDWSYGEPTEMLSPISSENFGLELGESSGAYIVRIPFGPRDKYIPKEHLWPHIQEFVDGALVHIMQMSKVLGEQIGSGQPVWPVVIHGHYADAGDSAALLSGALNVPMVFTGHSLGRDKLDQILKQGRQTRDEINATYKIMRRIEAEELCLDTSEIIITSTRQEIEQQWGLYDGFDLTMARKLRARIRRGVSCFGRYMPRMIAIPPGMEFSHIAPHDVDLDSEEGNGDGSGSPDPPIWADIMRFFSNPRKPMILALARPDPKKNITTLVKAFGEHRELRNLANLTLIMGNRDVIDEMSSTNAAVLTSALKLIDKYDLYGQVAYPKHHKQSEVPDIYRLAARTKGVFINCALVEPFGLTLIEAAAYGLPMVATRNGGPVDIHRVLDNGILVDPHNQNEIAEALYKLVSDKHLWSQCRQNGLKNIHKFSWPEHCQNYLARVVTLKPRHPRWQKNDVAAEISEADSPEDSLRDIHDISLNLKLSLDSEKSGSKEGNSNALRRHFEDAAQKLSGVNDIKKDVPGENGKWSSLRRRKHIIVIAVDSVQDADFVQVIKNIFEASRNERSSGAVGFVLSTARAISELHTLLISGGIEASDFDAFICNSGSDLCYPSSSSEDMLNPAELPFMIDLDYHSQIEYRWGGEGLRKTLIRWAAEKNKESGQKIFIEDEECSSTYCISFKVSNTAAAPPVKEIRRTMRIQALRCHVLYSHDGSKLNVIPVLASRSQALRYLYIRWGVELSNITVIVGECGDTDYEGLLGGVHKTIILKGSFNTAPNQVHANRSYSSQDVVSFDKQGIASIEGYGPDNLKSALRQFGILKD

(19) United States
(12) Patent Application Publication
(10) Pub. No.: US 2005/0251882 A1
(43) Pub. Date: Nov. 10, 2005

(54) Sucrose phosphate synthase nucleic acid molecules and uses therefor

(76) Inventors: Robert L. D'Ordine, Ballwin, MO (US); Stanton B. Dotson, Chesterfield, MO (US); Pamela J. Sisson, St. Louis, MO (US); Stephen M. Duff, St. Louis, MO (US)

Correspondence Address: MONSANTO COMPANY, 800 N. LINDBERGH BLVD., ATTENTION: G.P. WUELLNER, IP PARALEGAL, (E2NA), ST. LOUIS, MO, 63167, US

(21) Appl. No.: 10/336,263

(22) Filed: Jan. 3, 2003

Related U.S. Application Data
(60) Provisional application No. 60/345,378, filed on Jan. 3, 2002.
(60) Provisional application No. 60/355,421, filed on Feb. 6, 2002.

(51) Int. Cl. A01H 1/00; C12N 15/82; A01H 5/00; C12N 5/04; C07H 21/04

(52) U.S. Cl. 800/284; 800/320.1; 800/312; 536/23.2; 435/412; 435/415; 435/468

(57) ABSTRACT

The present invention relates generally to plant molecular biology and genetic engineering. In one embodiment, the present invention relates to isolated nucleic acids from cyanobacteria encoding sucrose phosphate synthase (SPS) or SPS-like proteins, in another embodiment, the present invention relates to isolated nucleic acids from maize plants encoding sucrose phosphate synthase (SPS) proteins. Each protein disclosed has utility in improving agronomic, horticultural and/or quality traits of plants, including yield.

### CROSS REFERENCE TO RELATED APPLICATIONS

This application claims benefit under 35USC § 119(e) of U.S. provisional application Ser. No. 60/345,378 filed Jan. 3, 2002, and U.S. provisional application Ser. No. 60/355,421 filed Feb. 6, 2002, both of which are herein incorporated by reference.

### FIELD OF THE INVENTION

The present invention relates generally to plant molecular biology and genetic engineering. In one embodiment, the present invention relates to isolated nucleic acids from cyanobacteria encoding sucrose phosphate synthase (SPS) or SPS-like proteins, in another embodiment, the present invention relates to isolated nucleic acids from maize plants encoding sucrose phosphate synthase (SPS) proteins. Each protein disclosed has utility in improving agronomic, horticultural and/or quality traits of plants, including yield.

### BACKGROUND OF THE INVENTION

One of the goals of plant genetic engineering is to produce plants with agronomic and horticultural traits of economic importance. Traits of particular interest may include high yield and improved quality. Although the yield from a plant is influenced by external environmental factors, the yield of the plant is also determined in part, by the export of sucrose from its source of production, i.e., leaves, to its sink, e.g., fruits and seeds, which are in turn determined by internal controlling factors. For example, enhancement of the yield of a plant may be achieved by genetically manipulating the plant's sucrose synthesis pathway so that the sucrose export is greatly increased. As a result, the intrinsic size of plant organs such as the fruit and seed, or the number of the fruits and seeds is increased.

A key enzyme of the cytoplasmic sucrose synthesis pathway is sucrose phosphate synthase (SPS) (Stitt et al., In: The Biochemistry of Plants, Hatch and Boardman eds, Academic Press, N.Y., p 327, 1987). This enzyme is found in photosynthetic tissues such as leaves and catalyzes the conversion of UDP-glucose and fructose-6-phosphate to UDP and sucrose-6-phosphate. SPS catalyzes what is thought to be the rate-limiting step in sucrose biosynthesis and as such has long been considered a target to increase sucrose synthesis. It is hypothesized that by increasing the expression of SPS in a plant, it will be possible to increase sucrose levels in the cells of that plant. Thus, an increase in sucrose biosynthesis will lead to an increase in sucrose export to the sink tissue and ultimately to an increase in yield. This increase could also lead to greater starch in leaves, creating larger source capacity for the plant.

Identification, isolation and characterization of SPS genes from different sources have significant impact on the effort to improve yield in desired crop plants. It is desirable that SPS cDNA and proteins from different sources be identified and characterized so that their specific functions in the sucrose biosynthetic pathway can be studied. Because there are numerous factors that can affect the expression and/or utility of a transgene, the identification of unique genes that code for proteins with different properties is useful for finding a gene or genes that have the desired effects. Important properties that need to be considered in the case of SPS and which may affect the expression or utility are allosteric effectors, substrate selectivity and Km, codon usage bias, size of the gene and protein-protein interactions. Therefore, identification, isolation, characterization and functional analysis of SPS genes from different species will help clarify their roles in sucrose biosynthesis and ultimately in plant growth and development. SPS nucleic acids and proteins have been identified from a cyanobacterium (Genbank accession No. gi1001295). Further effort in isolating cyanobacterial SPS nucleic acids from different sources would greatly benefit plant transformation process for desired yield improvement.

SPS nucleic acids and proteins have been identified from several higher plants. These plants include corn (Genbank accession No. CAA01354), tomato (Genbank accession No. AF071786), tobacco (Genbank accession No. AF194022_{—}1), spinach (Genbank accession No. AAA20092), potato (Genbank accession No. CAA51872), Craterostigma plantagineum (Genbank accession Nos. CAA7250 and CAA7249); sugarbeet (Genbank accession No. CAA57500), sugarcane (Genbank accession Nos. BAA19242 and BAA19241), Arabidopsis thaliana (Genbank accession No. CAB39764) and rice (Genbank accession No. AAC49379). In addition, SPS has also been isolated from a cyanobacterium species (Genbank Accession No. 1001295). Unique SPS genes that may exist in different species and that may have different regulation properties remain to be identified and their exact functions in the sucrose biosynthetic pathway studied. Further efforts on isolating and characterizing SPS nucleic acids from different sources, and different SPS genes from the same source, would be beneficial to advancing the science of plant biotechnology for desired yield improvement in crop plants.

### SUMMARY OF THE INVENTION

In accordance with the present invention, novel SPS and SPS-like genes from cyanobacteria species and Zea mays (maize or corn) are provided. The introduction and expression of these genes in plants provides a means for improving the qualities or characteristics of the resulting transformed plant, including improving the yield of the commercial commodity of the plant, e.g. seeds, fruit or leaf. Methods for using the isolated genes, proteins and fragments of the proteins for gene identification and analysis, preparation of transformation constructs and transformation of plant cells are also provided. The nucleic acids of the present invention from cyanobacterial species encoding SPS or SPS-like polypeptides are characterized by being smaller in size than SPS proteins of higher plants and have molecular weights ranging from about 46.5 kD to about 80.5 kD. The cyanobacterial SPS sequences of this invention are isolated from Anabaena sp., Prochlorococcus marinus, Nostoc punctiforme, and Synecochococcus sp. The SPS sequences of the invention isolated from maize, identified as an SPS genes and enzymes, are also unique in its physical characteristics from other SPS sequences from other higher plants and from the cyanobacterial genes and peptides.

In a preferred embodiment, an SPS gene of the present invention is introduced into a C4 plant such as maize under the transcriptional regulation of a promoter with specificity or significant preferential expression in the mesophyl tissue and cells of the C4 plant. Through preferential expression of the SPS protein in this tissue of the C4 plant, particularly maize, the yield of the plant, i.e seed, is enhanced and the quality characteristics of the seed are improved.

In one aspect of the present invention, an isolated nucleic acid from a cyanobacterium selected from the group consisting of Anabaena sp., Prochlorococcus marinus, Nostoc punctiforme, and Synechococcus sp is provided that comprises a nucleotide sequence, wherein the nucleotide sequence is defined as follows: (1) the nucleotide sequence encodes a polypeptide having an amino acid sequence that has at least about 50%, at least about 60%, at least about 70%, at least about 80%, at least about 90%, or at least about 95% sequence identity to a sequence selected from the group consisting of SEQ ID NOs: 2, 4, 6, 8, 10, 12 and 14; (2) the nucleotide sequence hybridizes under stringent conditions to the complement of a second isolated nucleic acid, wherein the nucleotide sequence of the second isolated nucleic acid encodes a polypeptide having an amino acid sequence selected from the group consisting of SEQ ID NOs: 2, 4, 6, 8, 10, 12 and 14; or (3) the nucleotide sequence is complementary to (1) or (2); wherein both polypeptides have the enzymatic activity of a sucrose phosphate synthase (SPS). Moreover, the sequence identity for certain SPS polypeptides to be within the scope of this invention may even be as low as about 35%, 40%, 45% or 50% identical as compared to Anabaena and Nostoc sp SPS peptides, and about 45% for Synechococcus sp. SPS peptides.

In a yet further aspect of the present invention, an isolated nucleic acid from a cyanobacterium selected from the group consisting of Anabaena sp., Prochlorococcus marinus, Nostoc punctiforme, and Synechococcus sp is provided that comprises a nucleotide sequence, wherein the nucleotide sequence is defined as follows: (1) the nucleotide sequence has at least about 55% or at least about 60% sequence identity (or about 70%, 80%, 90% or 95% sequence identity) to a sequence selected from the group consisting of SEQ ID NOs: 1, 3, 5, 7, 9, 11 and 13; (2) the nucleotide sequence hybridizes under stringent conditions to the complement of a second isolated nucleic acid, wherein the nucleotide sequence of the second isolated nucleic acid is selected from the group consisting of SEQ ID No: 1, 3, 5, 7, 9, 11, or 13; or (3) the nucleotide sequence is complementary to a nucleotide sequence described in (1) or (2).

Substantially purified polypeptides from a cyanobacterium selected from the group consisting of Anabaena sp., Prochlorococcus marinus, Nostoc punctiforme, and Synechococcus sp are further provided that comprise an amino acid sequence as described above and herein.

In another aspect of the preferred embodiment of the present invention, an isolated nucleic acid molecule is provided that comprises a nucleotide sequence or complement thereof, wherein the nucleotide sequence has mutations at locations that remove a phosphorylation site in the encoded SPS2 polypeptide. Said mutants created from an amino acid sequence comprising SEQ ID NO: 54, 56, 58, 60, 62, 64, 66, 68 or 70.

In still another aspect of the preferred embodiment of the present invention, an isolated nucleic acid molecule is provided that comprises a nucleotide sequence or complement thereof, wherein the nucleotide sequence has had its terminal sequence removed at position 486 for better expression in plants and encodes a truncated SPS polypeptide comprising an amino acid sequence having SEQ ID NO: 58.

In yet another aspect of the preferred embodiment, an isolated nucleic acid molecule from maize (Zea mays) is provided that comprises a nucleotide sequence, wherein the nucleotide sequence is defined as follows: (1) the nucleotide sequence encodes a polypeptide having an amino acid sequence that has at least about 60%, at least about 70%, or at least about 80%, at least about 90%, or at least about 95%, sequence identity to a sequence selected from the group consisting of SEQ ID NOs: 54, 56, 58, 60, 62, 64, 66, 68 and 70; (2) the nucleotide sequence hybridizes under stringent conditions to the complement of a second isolated nucleic acid, wherein the nucleotide sequence of the second isolated nucleic acid encodes a polypeptide having an amino acid sequence selected from the group consisting of SEQ ID NOs: 54, 56, 58, 60, 62, 64, 66, 68 and 70; or (3) the nucleotide sequence is complementary to (1) or (2), wherein both polypeptides have the enzymatic activity of a sucrose phosphate synthase (SPS).

In a further preferred embodiment of the present invention, an isolated nucleic acid molecule from maize (Zea mays) is provided that comprises a nucleotide sequence, wherein the nucleotide sequence is defined as follows: (1) the nucleotide has at least about 60%, at least about 70%, at least about 80%, at least about 90%, or at least about 95%, sequence identity to a sequence selected from the group consisting of SEQ ID NO: 53, 55, 57, 59, 61, 63, 65, 67, 69, 71; (2) the nucleotide sequence hybridizes under stringent conditions to the complement of a second isolated nucleic acid, wherein the nucleotide sequence of the second isolated nucleic acid is selected from the group consisting of SEQ ID No: 53, 55, 57, 59, 61, 63, 65, 67, 69, 71; or (3) the nucleotide sequence is complementary to (1) or (2).

In a still further preferred embodiment of the present invention, a substantially purified polypeptide from maize (Zea mays) is provided that comprises an amino acid sequence, wherein the amino acid sequence is defined as follows: (1) the amino acid sequence is encoded by a first nucleotide sequence which specifically hybridizes to the complement of a second nucleotide sequence selected from the group consisting of SEQ ID NO: 53, 55, 57, 59, 61, 63, 65, 67, 69, 71; (2) the amino acid sequence is encoded by a third nucleotide sequence that has at least about 60%, at least about 70%, at least about 80%, at least about 90%, or at least about 95%, sequence identity to a sequence selected from the group consisting of SEQ ID NO: 53, 55, 57, 59, 61, 63, 65, 67, 69, 71; or (3) the amino acid sequence has at least about 60%, at least about 70%, at least about 80%, at least about 90%, or at least about 95%, sequence identity to a sequence selected from the group consisting of SEQ ID NO: 54, 56, 58, 60, 62, 64, 66, 68, and 70.

In a still further embodiment of the present invention, recombinant DNA vectors are also provided for use in plant transformation for modification of the phenotypic characteristics of desired crop plants e.g., yield and quality enhancement. These vectors comprise regulatory elements useful in plants and a structural nucleotide sequence in accordance with the invention described herein encoding an SPS polypeptide wherein the polypeptide has the enzymatic activity of a sucrose phosphate synthase (SPS).

Transgenic plants produced and obtained in accordance with the inventions described herein are also provided. In one respect, these transgenic plants may exhibit an elevated sucrose production and thereafter export of such sucrose from its leaves or other area of origin to its reproductive organs for seed or fruit development and ultimate yield enhancement. In another respect these plants may have increased starch in their leaves. Increased starch is valuable to a plant as a stored energy source.

In a yet still further embodiment of the present invention, a method for overexpressing a SPS enzyme in a plant is also provided, comprising the steps of:
(a) inserting into the genome of a plant a nucleic acid sequence comprising in the 5′ to 3′ direction an operably linked recombinant, double-stranded DNA molecule, wherein the molecule comprises:
   (i) a promoter that functions in the cells of the plant,
   (ii) a structural DNA nucleic acid sequence that causes the production of an RNA sequence that encodes a SPS nucleic acid sequence set forth in SEQ ID NOs: 53, 55, 57, 59, 61, 63, 65, 67, 69, and 71, or a complement thereof,
   (iii) a 3′ non-translated DNA nucleic acid sequence that functions in the cells of the plant to cause termination of transcription;
(b) obtaining transformed plant cells containing the nucleic acid sequence of step (a); and
(c) regenerating from the transformed plant cells a genetically transformed plant that overexpresses the SPS enzymes in the transformed plant wherein the transformed plant demonstrates elevated sucrose production and export thereof.
   In a still further embodiment of the present invention, a method for obtaining an isolated nucleic acid molecule encoding all or a substantial portion of the amino acid sequence of a SPS or SPS-like polypeptide is also provided, the method comprising the steps of: (a) probing a cDNA or genomic library with a hybridization probe comprising a nucleotide sequence encoding all or a portion of the amino acid sequence of a polypeptide, wherein the amino acid sequence of the polypeptide is set forth in SEQ ID NOs: 54, 56, 58, 60, 62, 64, 66, 68 and 70 or the amino acid sequence of the polypeptide is set forth in SEQ ID NOs: 54, 56, 58, 60, 62, 64, 66, 68 and 70 with conservative amino acid substitutions; (b) identifying a DNA clone that hybridizes with the hybridization probe; (c) isolating the DNA clone identified in step (b); and (d) sequencing the cDNA or genomic fragment that comprises the clone isolated in step (c).

### BRIEF DESCRIPTION OF THE FIGURES

FIG. 1 shows an alignment of selected SPS proteins. Regions where phosphorylation sites are missing from cyanobacterial proteins are noted. Four sites reported in the literature are highlighted in bold type. Note that these sites are missing from the cyanobacterial species as well as some of the plant SPS genes in various locations. Also noted (gray box) are the positions where the cyanobacterial sequences differ from the recently published rules (Curatti et al., Planta, 211: 729-735, 2000) for SPS sequences.

FIG. 2 demonstrates SPS activity. FIG. 2a HPLC trace demonstrating formation of S6P under conditions as described in Example 7 below. FIG. 2b LC-MS analysis of the product confirming S6P.

FIG. 3 shows reduced Phosphate Inhibition of cyanobacterial genes. Filled circles represent Anabaena C154, filled squares Anabaena C287, and filled triangles Wheat SPS.

FIGS. 4a, b, and c. Codon usage comparison with Arabidopsis and corn.

FIG. 4a is a graph of comparison of codon usage of various cyanobacterial and algal species to the Arabidopsis genome. The graph represents the frequency of usage of a particular codon per 1000 codons. Codon usage that most closely matches with Arabidopsis is Anabaena.

FIGS. 4b and c are graphs that represent quite a bit of codon usage information, but from the stand point of finding those most similar to maize from the standpoint of codon-usage-level similarity, the nearest non-corn neighbor to a corn SPS gene is that of rice (Accession number, OJ990427_{—}03.9927.C15), and the nearest microbial neighbor is Anabaena SPS C154 followed by Synechocystis sp., strain PCC6803. Comparing the individual genes versus all genes currently available (in Genbank) for maize, rice is the closest species, followed by Anabaena. The genes had their codons counted and reduced to a vector of codon usage. Two distance functions were defined to measure the “distance” between two sequences: one based on codon usage, the other based on codon preference (usage being the frequency of that codon in a gene, preference being the relative frequency with which synonymous codons are used). The function represents the Euclidian distance between the codon usage/preference vectors. A third distance function was defined that represents the Euclidian distance combining the usage and preference distances. The notion is that codon usage reflects pressures in codon selection based on GC content, nucleotide availability, and codon preference is more likely affected by tRNA availability/stability. Graphs b and c represent the output from this analysis.

FIG. 5 shows cyanobacterial SPS gene comparison. Alignment highlights the similarities among the cyanobacterial proteins as well as the differences. Length of the genes is an obvious difference.

FIG. 6 shows a plasmid map, pMON63101, that is an E. coli expression vector containing Anabaena SPS C154 pET-28b.

FIG. 7 shows a plasmid map, pMON63102, that is an E. coli expression vector containing Anabaena SPS C287 pET-28b.

FIG. 8 shows a plasmid map, pMON63103, that is a binary vector for Agrobacterium-mediated transformation and constitutive expression genes in plants designed using pMON 23450. It contains Anabaena SPS C154.

FIG. 9 shows a plasmid map, pMON63104, that is a binary vector for Agrobacterium-mediated transformation and constitutive expression genes in plants designed using pMON 23450. It contains Anabaena SPS C287.

FIG. 10 shows a plasmid map, pMON63109, that is a binary vector for Agrobacterium-mediated transformation and constitutive expression genes in plants designed using pMON23450, that contains Anabaena SPS c287 no stop for C-Flag fusion.

FIG. 11 shows a plasmid map, pMON63110, that contains Anabaena SPS c287 with a C-histag in pET-28b.

FIG. 12 shows a plasmid map, pMON63111, that is a binary vector for Agrobacterium-mediated transformation and constitutive expression genes in plants containing Anabaena SPS c154 no stop for C-Flag fusion.

FIG. 13 shows a plasmid map, pMON63112, that contains Anabaena SPS c154 no stop with a C-Histag in pET-28b.

FIG. 14 shows a plasmid map, pMON63115, that is a protoplast transformation vector designed using pMON13912 for transient expression of genes in corn protoplasts that contains Anabaena SPS c287 no stop for C-Flag fusion.

FIG. 15 shows a plasmid map, pMON63116, that is protoplast transformation a vector for transient expression genes in protoplasts designed using pMON13912, that contains Anabaena SPS c154 no stop for C-Flag fusion.

FIG. 16 is nucleotide sequence comparison of maize SPS1 (GenBank Accession NO. g168625, maize sucrose phosphate synthase mRNA, complete cds coding region only) with maize SPS2. This figure shows gap of maize SPS 1 coding sequence only from 1 to 3207 and maize SPS2 coding sequence only from 1 to 3180.

FIG. 17 is a protein sequence comparison of maize SPS 1 and SPS2. This figure shows a gap comparison of maize SPS2 amino acid residues from 1 to 1059 and maize SPS 1 amino acid residues from 1 to 1068. BLOSUM62 amino acid substitution matrix was as in Henikoff and Henikoff (Proc. Natl. Acad. Sci. USA 89: 10915-10919; 1992).

FIG. 18 shows alignment of selected SPS proteins from cyanobacteria and higher plants. Regions where phosphorylation sites are missing from cyanobacterial proteins are noted. Four sites reported in the literature are highlighted in bold type. Note that these sites are missing from the cyanobacterial species as well as some of the plant SPS genes in various locations. Also noted (gray box) are the positions where the cyanobacterial sequences differ from the recently published rules for SPS sequences.

FIG. 19 is a summary of mutagenesis strategy for maize SPS2 subcloning.

FIG. 20 is a gap comparison of maize SPS2 and mutated maize SPS2 sequence. It shows gap of maize SPS2Mu nucleotides from 1 to 3180 (maize SPS2 from pMON52915 two point mutations) and maize SPS2 nucleotides from 1 to 3180.

FIG. 21 is an example chromatogram for tSPS2 activity in crude extracts under typical assay conditions (30 minutes). Y axis for trace is in uncorrected CPM. A typical control (second chromatogram), substrated only with extraction buffer added instead of enzyme, incubated and quenched under the same conditions, is included for comparison. Peak at 5.40-5.50 is S6P.

FIG. 22 shows a vector map designed for construction of other plant transformation vectors. The t-SPS2 gene is obtained from pMON52915 and is subcloned into pMON13912.

FIG. 23 shows a vector map for construction of other plant transformation vectors. This is produced from the vector in FIG. 7 and contains maize SPS2, the HSP 70 intron and 35S promoter.

FIG. 24 show a vector that is used to design plant transformation vectors containing any form of SPS2 gene sequences that may include, for example, a full-length, a truncated or a mutated SPS2 gene sequence behind specific promoter and intron combinations. Examples of the promoters to be used include PPDK and CAB or PPDK promoter alone for leaf mesophyll cell expression, and the 35S and e35S-SSP promoters for maize protoplast transformation.

FIG. 25 shows the activity of the SPS enzyme in delta 469 SPS events. As can be clearly seen, SPS activity is much higher in the leaves at all times, and increases significantly during the day.

FIG. 26 shows the sucrose levels in corn leaves. The events on the left with lower sucrose in leaves are having a silencing effect, and down-regulating the SPS activity. The rest of the events have higher levels of SPS and higher levels of sucrose in leaves.

FIG. 27 shows a western blot of several events showing the increased amount of SPS due to heterologous expression from the recombinant DNA construct incorporated into the genome of these plants.

FIG. 28 shows a comparison of active sites and regulatory regions from a series of SPS enzymes, please see examples for a complete discussion.

FIG. 29 shows a binary vector, pMON66105, which was made for over-expressing maize SPS1 gene in soybean under leaf specific promoter SSU. PMON66105 is a 2 T-DNA vector, where the selectable marker expression cassette [P-FMV/HSP70/CTP2/CP4/E9] and the SPS 1 expression cassette [SSU/mSPS/E9] are on two separate T-DNA's contained on a single binary vector.

### DETAILED DESCRIPTION OF THE PREFERRED EMBODIMENTS

The present invention relates to the isolation of a series of novel SPS enzymes from cyanobacteria and corn. The expansion of the class of enzymes known as sucrose phosphate synthase (SPS) leads to novel utilities as each gene is expected to have novel biochemical profiles (Km, etc.). Those from corn will also be expected to be evolutionarily selected for novel uses within the plant, i.e. significantly different expression profiles. These profiles include unique expression patterns during development, differing tissue specificity, and different expression profiles due to environmental stimuli (including light, heat, cold, drought, etc.).

The present invention is based, in part, on the isolation and characterization of nucleic acids encoding SPS proteins from cyanobacteria including Anabaena sp., Prochlorococcus marinus, Nostoc punctiforme and Synechococcus sp. These isolated SPS nucleic acid sequences share very low sequence identity in comparison with other known SPS sequences from other cyanobacteria, e.g., Synechocystis sp., and higher plants, e.g., corn (Table 1). The present invention relates to isolated and characterized SPS or SPS-like sequences that differ with published assertions about invariant residues for SPS proteins (Curatti et al., Planta 211: 729-735, 2000). The Anabaena SPS cDNA also has a codon usage that is most amenable to expression in higher plants such as corn.

TABLE 1
Gap comparison of coding SPS nucleotide sequences to Maize SPS1 and
Synechocystis SPS as reference.

| | | | | Reference for |
|---|---|---|---|---|
| Nucleotide Sequence 1 | Nucleotide Sequence 2 | Similarity % | Identity % | sequence 2 |
| Synechocystis SPS | Anabaena SPSc154 | 43 | 43 | SEQ ID NO: 1 |
| Synechocystis SPS | Anabaena SPSc287 | 44 | 44 | SEQ ID NO: 3 |
| Synechocystis SPS | Nostoc C603 | 41 | 41 | SEQ ID NO: 9 |
| Synechocystis SPS | Nostoc C599 | 43 | 43 | SEQ ID NO: 7 |
| Synechocystis SPS | Nostoc C621 | 43 | 43 | SEQ ID NO: 11 |
| Synechocystis SPS | Synechcoccus C261 | 49 | 49 | SEQ ID NO: 13 |
| Synechocystis SPS | Prochlorococcus SPS | 52 | 52 | SEQ ID NO: 5 |
| Synechocystis SPS | Maize SPS1 | 47 | 47 | g168625 |
| Maize SPS1 | Synechocystis SPS | 47 | 47 | 1001295 |
| Maize SPS1 | Anabaena SPSc154 | 40 | 40 | SEQ ID NO: 1 |
| Maize SPS1 | Anabaena SPSc287 | 37 | 37 | SEQ ID NO: 3 |
| Maize SPS1 | Nostoc C603 | 39 | 39 | SEQ ID NO: 9 |
| Maize SPS1 | Nostoc C599 | 37 | 37 | SEQ ID NO: 7 |
| Maize SPS1 | Nostoc C621 | 39 | 39 | SEQ ID NO: 11 |
| Maize SPS1 | Synechcoccus C261 | 48 | 48 | SEQ ID NO: 13 |
| Maize SPS1 | Prochlorococcus SPS | 47 | 47 | SEQ ID NO: 5 |

The present invention also relates to SPS polypeptides substantially purified from cyanobacteria that are unique in many characteristics. All of them are small in size (Table 2) relative to SPS nucleic acid sequences of higher plants including corn (Genbank accession No. CAA01354), tomato (Genbank accession No. AF071786), tobacco (Genbank accession No. AF194022_{—}1), spinach (Genbank accession No. AAA20092), potato (Genbank accession No. CAA51872), Craterostigma plantagineum (Genbank accession Nos. CAA7250 and CAA7249); sugarbeet (Genbank accession No. CAA57500) sugarcane (Genbank accession Nos. BAA19242 and BAA19241), Arabidopsis thaliana (Genbank accession No. CAB39764), and rice (Genbank accession No. AAC49379). Additionally some are smaller than even other cyanobacterial genes, e.g., Synechocystis (Genbank accession No. gi1001295). These cyanobacterial SPS proteins, unlike SPS proteins of other higher plants as mentioned above, do not contain regulatory phosphorylation sites.

TABLE 2
Molecular weights of the cyanobacterial SPS polypeptide
sequences of the present invention and those of the SPS
polypeptide sequences from Synechocystis and higher plants

| | SEQ ID NO or | | |
|---|---|---|---|
| | GenBank | | Molecular |
| | Accession | No. of amino acid | weight |
| Organisms | No. | residues in sequence | (kDa) |
| Anabaena c154 | SEQ ID NO: 1 | 425 | 47.2 |
| Anabaena c287 | SEQ ID NO: 3 | 422 | 46.8 |
| Nostoc C603 | SEQ ID NO: 9 | 423 | 46.7 |
| Nostoc C599 | SEQ ID NO: 7 | 480 | 53.2 |
| Nostoc C621 | SEQ ID NO: 11 | 422 | 426 |
| Synechcoccus C261 | SEQ ID NO: 13 | 710 | 80.2 |
| Prochlorococcus | SEQ ID NO: 5 | 470 | 53.3 |
| Synechocystis | 1001295 | 720 | 81.4 |
| corn | CAA01354 | 1068 | 118.6 |
| spinach | AAA20092 | 1056 | 117.7 |
| tomato | AF071786 | 960 | 108.6 |
| rice | AAC49379 | 1049 | 116.5 |
| potato | CAA51872 | 1053 | 118.3 |
| tobacco | AF194022_1 | 1054 | 118.7 |
| Arabidopsis | CAB39764 | 1083 | 122.7 |
| thaliana | | | |

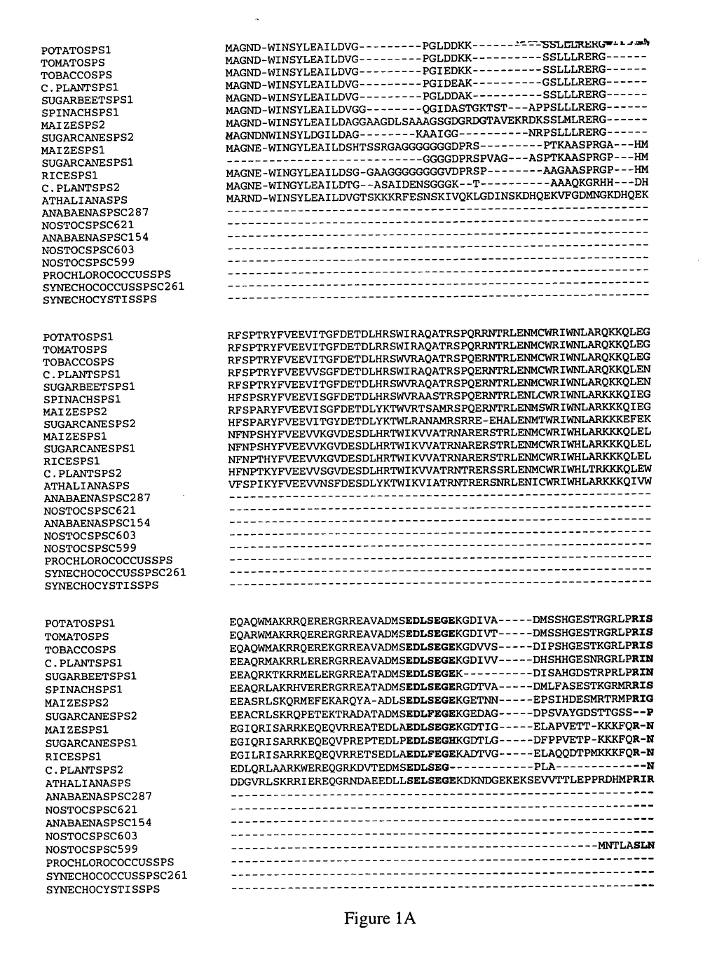

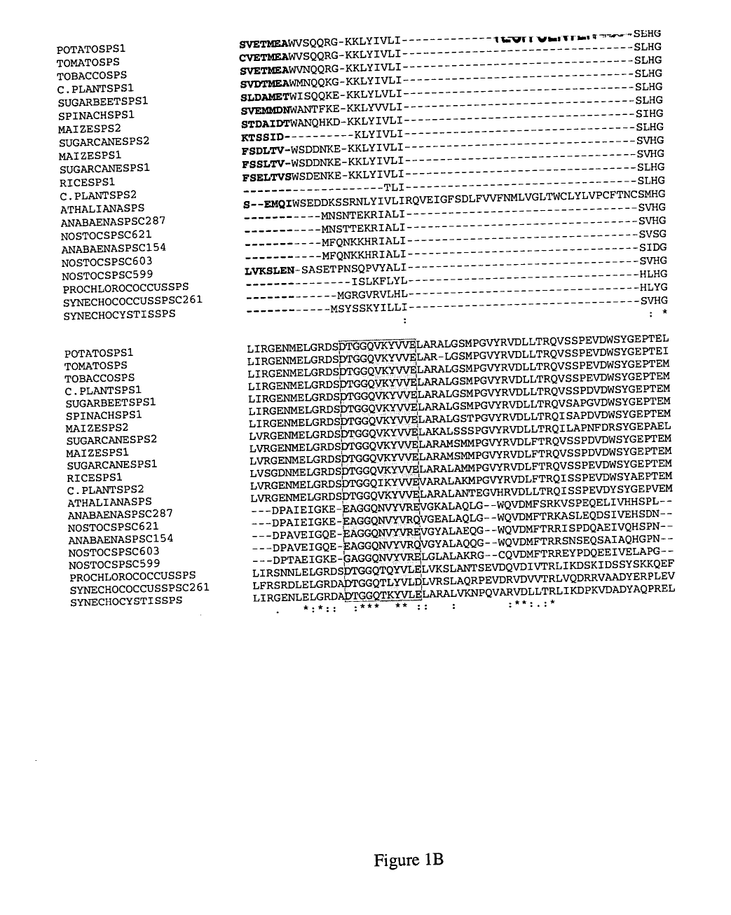

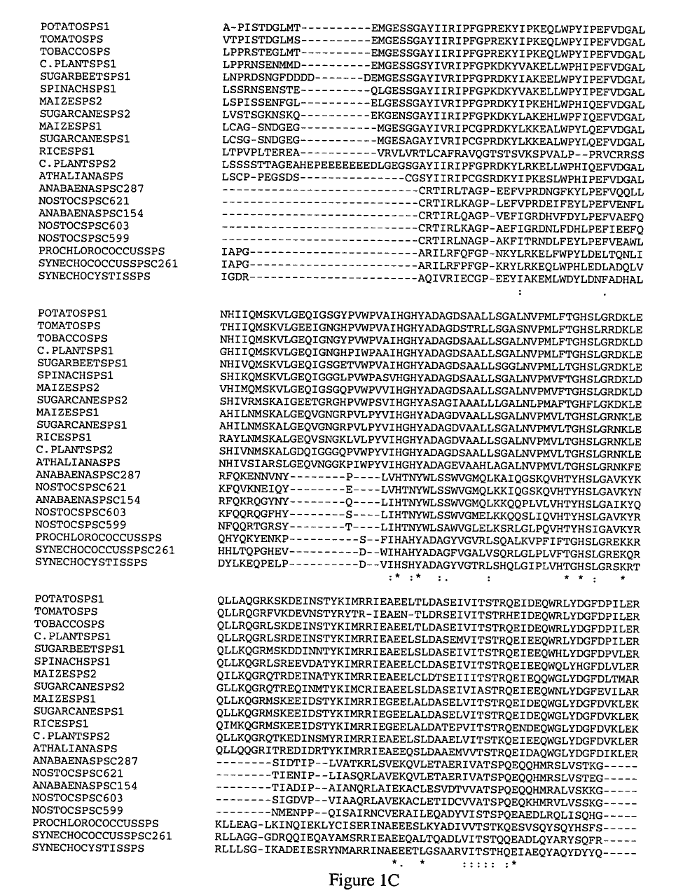

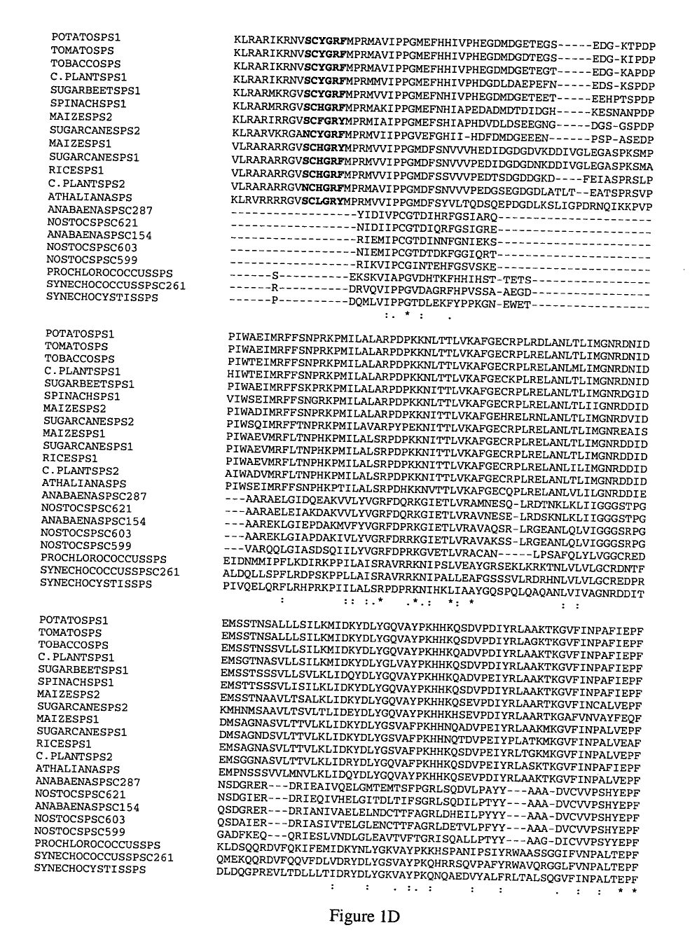

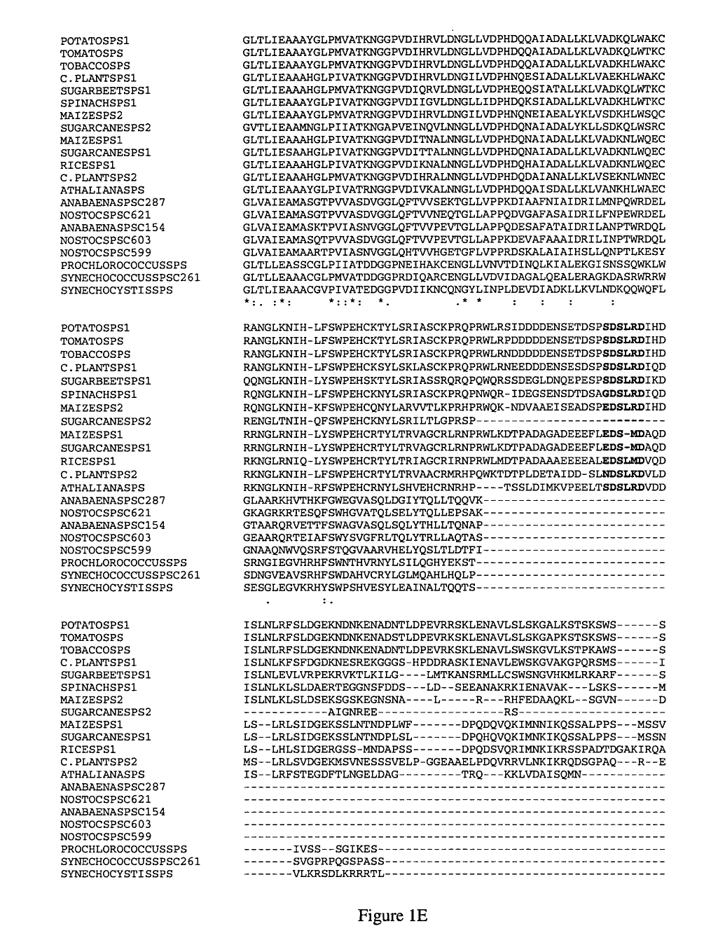

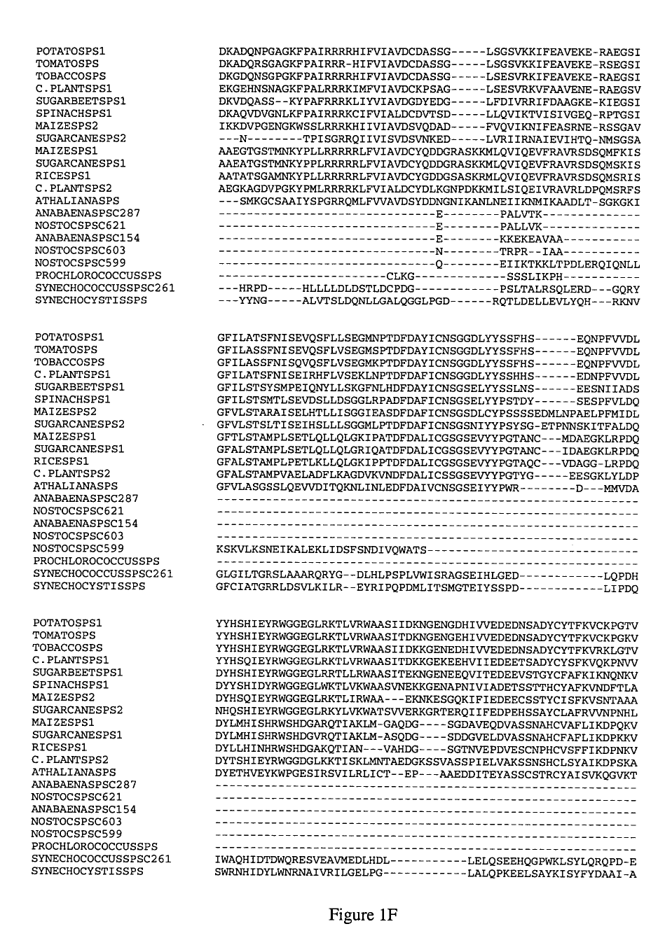

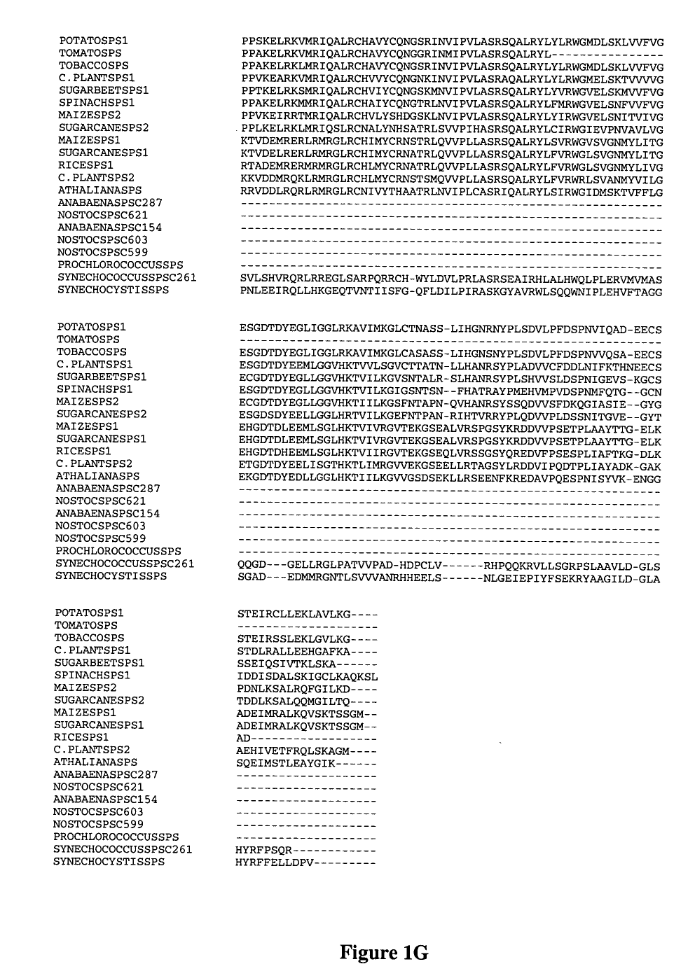

The polypeptides encoded by these SPS nucleic acids disclosed herein, i.e., these polypeptide sequences having SEQ ID NOs. 2 and 4 from Anabaena sp., SEQ ID NO. 6 from Prochlorococcus marinus, SEQ ID NOs. 8, 10 and 12 from Nostoc punctiforme and SEQ ID NO. 14 from Synechococcus sp., share significantly low amino acid sequence identity to those of higher plants (FIG. 1 and Table 3). The SPS polypeptides of the present invention even show significant amino acid sequence difference from other cyanobacterial SPS sequences. For example, the SPS amino acid sequences of Nostoc (c599) of the present invention show only 27% sequence identity to that of Synechocystis based upon the gap comparison method (Table 3).

The present invention has allowed further identification of other SPS genes that are distantly related to plants that would not otherwise be readily identified via sequence homology alone. These genes and others found by employing the methods disclosed in the present invention represent a novel set of SPS enzymes that have particular value, given their reduced sizes, favorable codon usage, and regulatory properties.

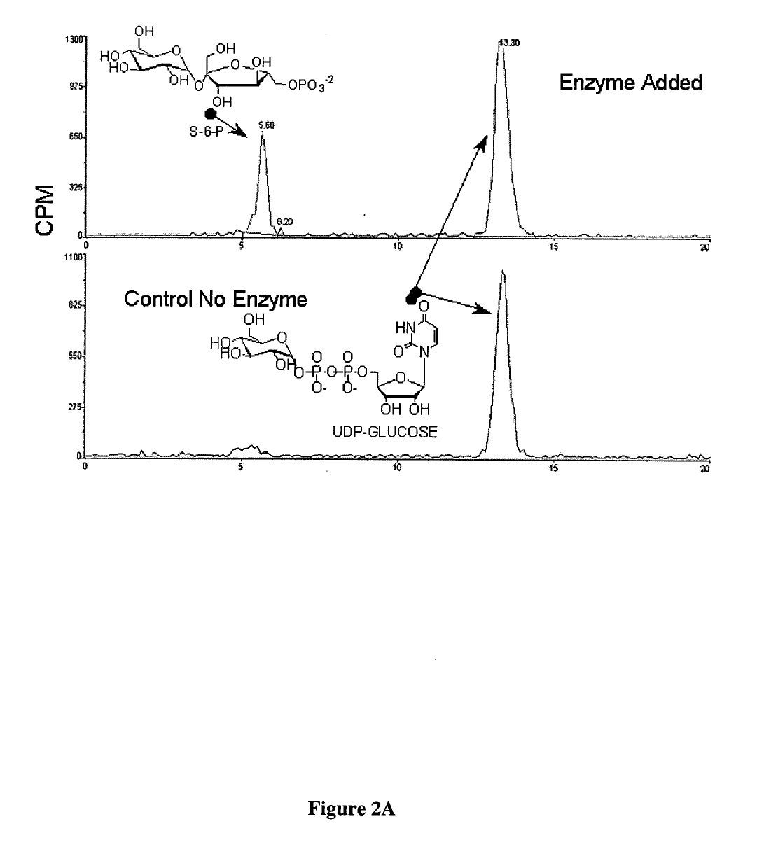

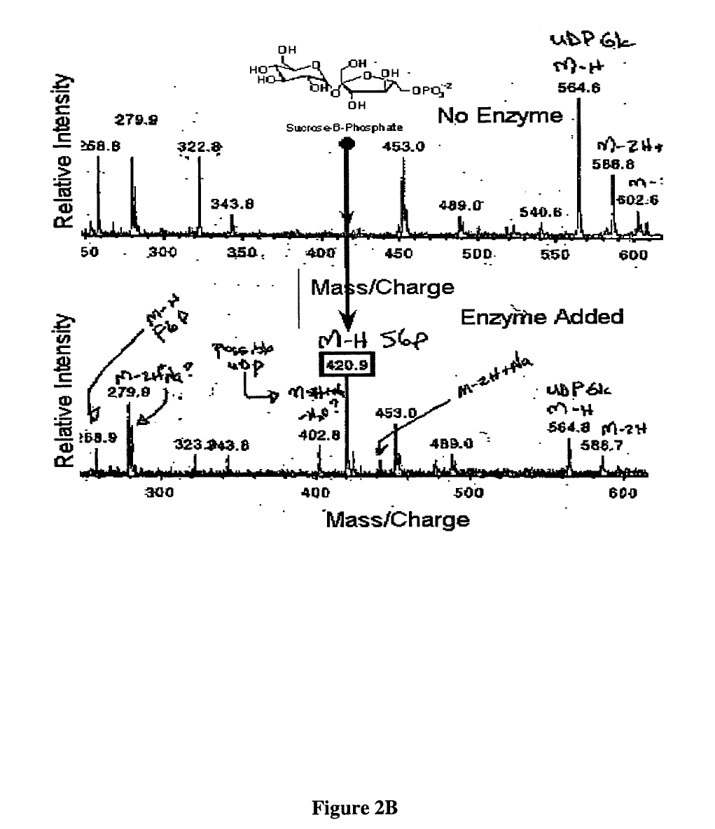

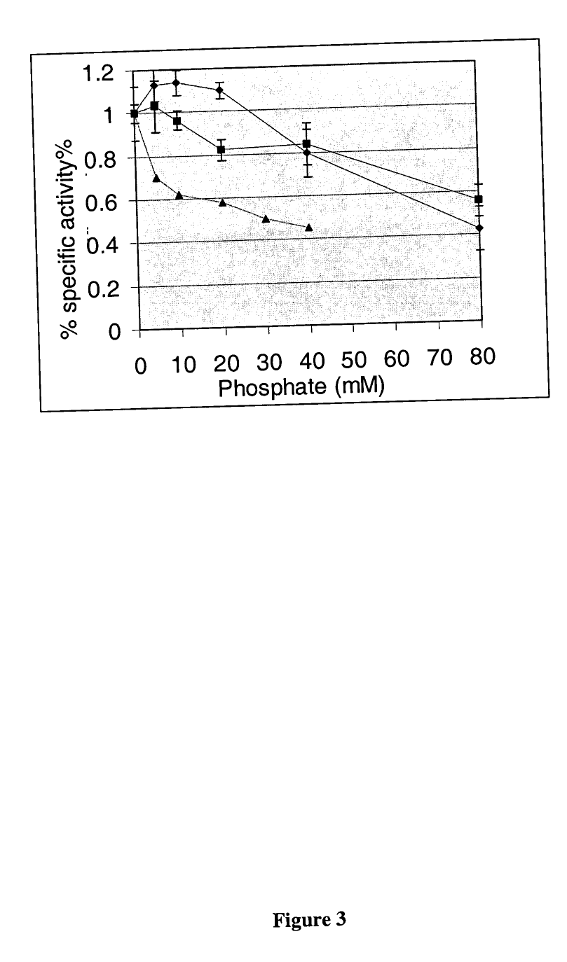

The present invention is based, in part, on the isolation and characterization of nucleic acids encoding SPS enzymes from maize (Zea mays). The following discussion is but one example of those isolated enzymes and is shown here as an example. Please see the examples and sequence listing for the full disclosure of the novel SPS enzymes isolated from this crop plant. The present invention relates to the SPS2 polypeptide isolated from maize that is unique in many characteristics. The SPS2 shares about 55% amino acid sequence with that of the maize SPS1 gene (GenBank Accession No. CAA01354, see Table 1, FIGS. 1 and 2). In addition, the isolated SPS2 gene of the present invention has less than 67% amino acid sequence identity to those of SPS proteins from other higher plants such as corn (GenBank Accession No. CAA01354), soybean (GenBank Accession No. Y11795)), Catalpa (GenBank Accession No. AB001338), spinach (GenBank Accession No. AAA20092), potato (GenBank Accession No. CAA51872), tomato (GenBank Accession No. AF071786), tobacco (GenBank Accession No. AF194022_{—}1), rice (GenBank Accession No. AAC49379) and Arabidopsis thaliana (GenBank Accession No. CAB39764) (Table 1; FIG. 3 for sequence alignments). In comparison with SPS proteins of cyanobacteria (GenBank Accession No. 1001295), SPS2 is larger in size (FIG. 3), and shares less than 43% sequence identity to those cyanobacterial SPS amino acid sequences (Table 2). The GAP comparison was made using the Blast algorithm (Altschul et al., Nucleic Acids Res. 25: 3389-3402, 1997).

TABLE 3
GAP comparison of maize SPS2 and SPS proteins of other higher plants.

| | | Identity | Similarity | Accession |
|---|---|---|---|---|
| Sequence 1 | Sequence 2 | (%) | (%) | No. |
| MAIZE SPS2 | C. PLANT SPS1 | 66 | 77 | CAA72506 |
| MAIZE SPS2 | TOBACCO SPS | 65 | 78 | AF194022_1 |
| MAIZE SPS2 | POTATO SPS1 | 65 | 78 | CAA51872 |
| MAIZE SPS2 | SPINACH SPS1 | 66 | 79 | AAA20092 |
| MAIZE SPS2 | SUGARBEET | 66 | 77 | CAA57500 |
| | SPS1 | | | |
| MAIZE SPS2 | Tomato SPS | 65 | 77 | AF071786 |
| MAIZE SPS2 | Sugarcane SPS2 | 58 | 72 | BAA19242 |
| MAIZE SPS2 | MAIZE SPS1 | 55 | 70 | CAA01354 |
| MAIZE SPS2 | C. PLANT SPS2 | 53 | 68 | CAA72491 |
| MAIZE SPS2 | Sugarcane SPS1 | 54 | 70 | BAA19241 |
| MAIZE SPS2 | Athaliana SPS | 51 | 66 | CAB39764 |
| MAIZE SPS2 | RICESPS1 | 51 | 67 | AAC49379 |

TABLE 4
GAP comparison of maize SPS2 and cyanobacterial SPS proteins.

| | | | | Accession, |
|---|---|---|---|---|
| | | Identity | Similarity | EST ID or |
| Sequence 1 | Sequence 2 | % | % | contig |
| Maize SPS2 | Synechocystis SPS | 41 | 53 | gil10012951 |
| | | | | dbjlBAA107 |
| | | | | 82.1l |
| Maize SPS2 | Anabaena SPSc154 | 31 | 43 | C154* |
| Maize SPS2 | Anabuena SPSc287 | 28 | 40 | C287 |
| Maize SPS2 | Nostoc C603 | 26 | 37 | C603 |
| Maize SPS2 | Nostoc C599 | 31 | 41 | C599 |
| Maize SPS2 | Nostoc C621 | 28 | 40 | C621 |
| Maize SPS2 | Synechococcus C261 | 37 | 50 | C261 |
| Maize SPS2 | Prochlorococcus SPS | 42 | 53 | C34 |

The SPS gene isolated from maize in the present invention has been demonstrated to be an SPS2 enzyme based upon an activity analysis. The present invention has allowed further identification of other SPS genes that are distantly related to plants that would not otherwise be identified via sequence homology alone.

The present invention includes a series of SPS enzymes isolated from corn. These include the SPS enzyme discussed above as well as a series of other enzymes isolated by the provided methods (see examples).

Isolated Nucleic Acids of the Present Invention

The term “nucleic acid” refers to a single or double-stranded polymer of deoxyribonucleotide or ribonucleotide bases read from the 5′ to the 3′ end. Nucleic acids may also optionally contain synthetic, non-natural or altered nucleotide bases that permit correct read through by a polymerase and do not alter expression of a polypeptide encoded by that nucleic acid.

An “isolated nucleic acid” refers to a nucleic acid that is no longer accompanied by those materials with which it is associated in its natural state or to a nucleic acid the structure of which is not identical to that of any of naturally occurring nucleic acid. Examples of an isolated nucleic acid include: (1) DNAs that have the sequence of part of a naturally occurring genomic DNA molecules but are not flanked by two coding sequences that flank that part of the molecule in the genome of the organism in which it naturally occurs; (2) a nucleic acid incorporated into a vector or into the genomic DNA of a prokaryote or eukaryote in a manner such that the resulting molecule is not identical to any naturally occurring vector or genomic DNA; (3) a separate molecule such as a cDNA, a genomic fragment, a fragment produced by polymerase chain reaction (PCR), or a restriction fragment; (4) recombinant DNAs; and (5) synthetic DNAs. An isolated nucleic acid may also be comprised of one or more segments of cDNA, genomic DNA or synthetic DNA.

It is also contemplated by the inventors that the isolated nucleic acids of the present invention also include known types of modifications, for example, labels which are known in the art, methylation, “caps”, substitution of one or more of the naturally occurring nucleotides with an analog. Other known modifications include internucleotide modifications, for example, those with uncharged linkages (methyl phosphonates, phosphotriesters, phosphoamidates, carbamates, etc.) and with charged linkages (phosphorothioates, phosphorodithioates, etc.), those containing pendant moieties, such as, proteins (including nucleases, toxins, antibodies, signal peptides, poly-L-lysine, etc.), those with intercalators (acridine, psoralen, etc.), those containing chelators (metals, radioactive metals, boron, oxidative metals, etc.), those containing alkylators, and those with modified linkages.

The term “nucleotide sequence” refers to both the sense and antisense strands of a nucleic acid as either individual single strands or in the duplex. It includes, but is not limited to, self-replicating plasmids, chromosomal sequences, and infectious polymers of DNA or RNA. “Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. A nucleotide sequence is said to be a “complement” of another nucleotide sequence if it exhibits complete complementarity. As used herein, molecules are said to exhibit “complete complementarity” when every nucleotide of one of the sequences is complementary to a nucleotide of the other.

A “coding sequence” or “structural nucleotide sequence” is a nucleotide sequence that is translated into a polypeptide, usually via mRNA, when placed under the control of appropriate regulatory sequences. The boundaries of the coding sequence are determined by a translation start codon at the 5′-terminus and a translation stop codon at the 3′-terminus. A coding sequence may include, but may not be limited to, genomic DNA, cDNA, and recombinant nucleotide sequences.

One skilled in the art will recognize that the SPS or SPS-like polypeptides of the invention, like other proteins, have different domains that perform different functions. Thus, the coding sequences need not be full length, so long as the desired functional domain of the protein is expressed. The distinguishing features of SPS or SPS-like polypeptides are discussed in detail in this section and in Examples.

The term “polypeptide” or “protein”, as used herein, refers to a polymer composed of amino acids connected by peptide bonds. The term “polypeptide” or “protein” also applies to any amino acid polymers in which one or more amino acid residue is an artificial chemical analogue of a corresponding naturally occurring amino acid, as well as to any naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. It is well known in the art that proteins or polypeptides may undergo modification, including but not limited to, disulfide bond formation, gamma-carboxylation of glutamic acid residues, glycosylation, lipid attachment, phosphorylation, oligomerization, hydroxylation and ADP-ribosylation. Exemplary modifications are described in most basic texts, such as, for example, Proteins—Structure and Molecular Properties, 2nd ed. (Creighton, Freeman and Company, N.Y., 1993). Many detailed reviews are available on this subject, such as, for example, those provided by Wold (In: Post-translational Covalent Modification of Proteins, Johnson, Academic Press, N.Y., pp. 1-12, 1983), Seifter et al. (Meth. Enzymol. 182: 626, 1990) and Rattan et al. (Ann. N.Y. Acad. Sci. 663:48-62, 1992). Modifications can occur anywhere in a polypeptide, including the peptide backbone, the amino acid side chains and the amino or carboxyl termini. In fact, blockage of the amino or carboxyl group in a polypeptide, or both, by a covalent modification, is common in naturally occurring and synthetic polypeptides and such modifications may be present in polypeptides of the present invention, as well. For instance, the amino terminal residue of polypeptides made in E. coli or other cells, prior to proteolytic processing, almost invariably will be N-formylmethionine. During post-translational modification of the polypeptide, a methionine residue at the NH₂ terminus may be deleted. Accordingly, this invention contemplates the use of both the methionine containing and the methionine-less amino terminal variants of the protein of the invention. Thus, as used herein, the term “protein” or “polypeptide” includes any protein or polypeptide that is modified by any biological or non-biological process. The terms “amino acid” and “amino acids” refer to all naturally occurring amino acids and, unless otherwise limited, known analogs of natural amino acids that can function in a similar manner as naturally occurring amino acids. This definition is meant to include norleucine, ornithine, homocysteine, and homoserine.

The term “enzymatic activity” of an enzyme refers to the enzyme's catalytic activity under appropriate conditions under which the enzyme serves as a protein catalyst that converts specific substrates to specific products. For the purpose of the present invention, the enzymatic activity of a sucrose phosphate synthase is defined by the production of one molecule of sucrose 6 phosphate and one molecule of UDP from one molecule of UDP-glucose and one molecule of fructose 6 phosphate. Magnesium ion (Mg²⁺) may be a cofactor in the enzymatic process. Some forms of the cyanobacterial SPS enzymes are not specific for the source of the glucosyl carrier molecule and will utilize, e.g. ADP-glucose (theoretically GDP-glucose, TDP-glucose, and/or CDP-glucose) as a substrate to produce ADP and sucrose-6-phosphate. It appears however that all true SPS enzymes may utilize fructose 6 phosphate and fructose will not serve as a substrate. Thus, the broad definition of SPS enzymatic activity may be the catalytic activity of the SPS enzyme in a catalytic process during which a nucleoside glucosyl carrier (ADP, UDP, GDP, TDP or CDP glucose) and a fructose-6 phosphate are converted to a sucrose 6 phosphate and a XDP (ADP, UDP, GDP, TDP or CDP).

The term “recombinant DNAs” refers to DNAs that contains a genetically engineered modification through manipulation via mutagenesis, restriction enzymes, and the like. The term “synthetic DNAs” refers to DNAs assembled from oligonucleotide building blocks that are chemically synthesized using procedures known to those skilled in the art. These building blocks are ligated and annealed to form DNA segments that are then enzymatically assembled to construct the entire DNA. “Chemically synthesized”, as related to a sequence of DNA, means that the component nucleotides were assembled in vitro. Manual chemical synthesis of DNA may be accomplished using well-established procedures, or automated chemical synthesis can be performed using one of a number of commercially available machines.

The term “substantially purified polypeptide” or “substantially purified protein”, as used herein, refers to a polypeptide or protein that is separated substantially from all other molecules normally associated with it in its native state and is the predominant species present in a preparation. A substantially purified molecule may be greater than 60% free, preferably 70% free, more preferably 80% free, more preferably 90% free, and most preferably 95% free from the other molecules (exclusive of solvent) present in the natural mixture. A substantially purified polypeptide may be obtained, for example, by extraction from a natural source (for example, a cyanobacterial cell); by expression of a recombinant nucleic acid encoding a SPS 1 polypeptide; or by chemically synthesizing the protein. Purity can be measured by any appropriate method, for example, column chromatography, polyacrylamide gel electrophoresis, or by HPLC.

The term “substantially identical” or “substantial identity” as reference to two amino acid sequences or two nucleotide sequences means that one amino acid sequence or nucleotide sequence has at least 60% sequence identity compared to the other amino acid sequence or nucleotide sequence as a reference sequence using the Gap program in the WISCONSIN PACKAGE version 10.0-UNIX from Genetics Computer Group, Inc. based on the method of Needleman and Wunsch (J. Mol. Biol. 48:443-453, 1970) using the set of default parameters for pairwise comparison (for amino acid sequence comparison: Gap Creation Penalty=8, Gap Extension Penalty=2; for nucleotide sequence comparison: Gap Creation Penalty=50; Gap Extension Penalty=3).

Polypeptides that are “substantially similar” share sequences as described above except that residue positions that are not identical may differ by conservative amino acid changes. Conservative amino acid substitutions refer to the interchangeability of residues having similar side chains. “Conservative amino acid substitutions” refer to substitutions of one or more amino acids in a native amino acid sequence with another amino acid(s) having similar side chains, resulting in a silent change. Conserved substitutes for an amino acid within a native amino acid sequence can be selected from other members of the group to which the naturally occurring amino acid belongs. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acid substitution groups are: valine-leucine, valine-isoleucine, phenylalanine-tyrosine, lysine-arginine, alanine-valine, aspartic acid-glutamic acid, and asparagine-glutamine.

One skilled in the art will recognize that the values of the above substantial identity of nucleotide sequences can be appropriately adjusted to determine corresponding sequence identity of two nucleotide sequences encoding the proteins of the present invention by taking into account codon degeneracy, conservative amino acid substitutions, reading frame positioning and the like. Substantial identity of nucleotide sequences for these purposes normally means sequence identity of at least about 35%, preferably at least about 50%, more preferably at least about 70%, and most preferably at least about 90%.

The term “codon degeneracy” refers to divergence in the genetic code permitting variation of the nucleotide sequence without affecting the amino acid sequence of an encoded polypeptide. The skilled artisan is well aware of the “codon-bias” exhibited by a specific host cell in usage of nucleotide codons to specify a given amino acid. Therefore, when synthesizing a gene for ectopic expression in a host cell, it is desirable to design the gene such that its frequency of codon usage approaches the frequency of preferred codon usage of the host cell.

Each of the nucleic acid encoding a SPS or SPS-like polypeptide of the present invention may be combined with other non-native, or “heterologous” sequences in a variety of ways. By “heterologous” sequences it is meant any sequence that is not naturally found joined to the nucleotide sequence encoding SPS or SPS-like polypeptide, including, for example, combinations of nucleic acid sequences from the same plant which are not naturally found joined together, or the two sequences originate from two different species.

In another aspect, the present invention provides an isolated nucleic acid comprising a structural nucleotide sequence and operably linked regulatory sequences, wherein the structural nucleotide sequence encodes a polypeptide having an amino acid sequence that is substantially identical to any SPS disclosed herein (dor example, see examples and sequence listing).

The term “operably linked”, as used in reference to a regulatory sequence and a structural nucleotide sequence, means that the regulatory sequence causes regulated expression of the operably linked structural nucleotide sequence.

“Expression” refers to the transcription and stable accumulation of sense or antisense RNA derived from the nucleic acid of the present invention. Expression may also refer to translation of mRNA into a polypeptide. “Sense” RNA refers to RNA transcript that includes the mRNA and so can be translated into polypeptide or protein by the cell. “Antisense” RNA refers to a RNA transcript that is complementary to all or part of a target primary transcript or mRNA and that blocks the expression of a target gene (U.S. Pat. No. 5,107,065). The complementarity of an antisense RNA may be with any part of the specific gene transcript, i.e., at the 5′ non-coding sequence, 3′ non-translated sequence, introns, or the coding sequence. “RNA transcript” refers to the product resulting from RNA polymerase-catalyzed transcription of a DNA sequence. When the RNA transcript is a perfect complementary copy of the DNA sequence, it is referred to as the primary transcript or it may be a RNA sequence derived from post-transcriptional processing of the primary transcript and is referred to as the mature RNA.

The term “overexpression” refers to the expression of a polypeptide or protein encoded by an exogenous nucleic acid introduced into a host cell, wherein said polypeptide or protein is either not normally present in the host cell, or wherein said polypeptide or protein is present in said host cell at a higher level than that normally expressed from the endogenous gene encoding said polypeptide or protein.

By “ectopic expression” it is meant that expression of a nucleic acid molecule encoding a polypeptide in a cell type other than a cell type in which the nucleic acid molecule is normally expressed, at a time other than a time at which the nucleic acid molecule is normally expressed or at a expression level other than the level at which the nucleic acid molecule normally is expressed.

“Antisense inhibition” refers to the production of antisense RNA transcripts capable of suppressing the expression of the target protein. “Co-suppression” refers to the production of sense RNA transcripts capable of suppressing the expression of identical or substantially similar foreign or endogenous genes (U.S. Pat. No. 5,231,020).

The term “gene” refers to the segment of DNA that is involved in producing a protein. Such segment of DNA includes regulatory sequences preceding (5′ non-coding sequences) and following (3′ non-coding sequences) the coding region as well as intervening sequences (introns) between individual coding segments (exons). A “Native gene” refers to a gene as found in nature with its own regulatory sequences. “Chimeric gene” refers any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

“Regulatory sequences” refer to nucleotide sequences located upstream (5′ non-coding sequences), within, or downstream (3′ non-translated sequences) of a structural nucleotide sequence, and which influence the transcription, RNA processing or stability, or translation of the associated structural nucleotide sequence. Regulatory sequences may include promoters, translation leader sequences, and polyadenylation recognition sequences.

As used herein, the term “mesophyll tissue” refers to ground tissue (parenchyma) of a leaf and the term “mesophyll cells” refer to the cells that comprise the mesophyll tissue. The mesophyll cells are located between the layers of epidermis and generally contain chloroplasts. In a C4 monocot plant such as a maize plant, the mesophyll cells are located around large bundle-sheath cells, forming two concentric layers around the vascular bundle. This unique wreathlike arrangement is referred as “Kranz anatomy” and is found in leaves of C4 plants.

The “translation leader sequence” refers to a DNA sequence located between the promoter sequence of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences have been described (Turner and Foster, Molecular Biotechnology 3: 225, 1995).

The “3′non-translated sequences” refer to DNA sequences located downstream of a structural nucleotide sequence and include sequences encoding polyadenylation and other regulatory signals capable of affecting mRNA processing or gene expression. The polyadenylation signal functions in plants to cause the addition of polyadenylate nucleotides to the 3′ end of the mRNA precursor. The polyadenylation sequence can be derived from the natural gene, from a variety of plant genes, or from T-DNA. An example of the polyadenylation sequence is the nopaline synthase 3′ sequence (NOS 3′; Fraley et al., Proc. Natl. Acad. Sci. USA 80: 4803-4807, 1983). Ingelbrecht et al. (Plant Cell 1: 671-680, 1989) exemplified the use of different 3′ non-translated sequences.

The isolated nucleic acids of the present invention may also include introns. Generally, optimal expression in monocotyledonous and some dicotyledonous plants is obtained when an intron sequence is inserted between the promoter sequence and the structural gene sequence or, optionally, may be inserted in the structural coding sequence to provide an interrupted coding sequence. An example of such an intron sequence is the HSP 70 intron described in PCT Publication WO 93/19189.

The laboratory procedures in recombinant DNA technology used herein are those well known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturer's specifications. These techniques and various other techniques are generally performed according to Sambrook et al., Molecular Cloning—A Laboratory Manual, 2nd. Ed., Cold Spring Harbor Laboratory, Cold Spring Harbor, N.Y. (1989).

Another aspect of the present invention relates to an isolated nucleic acid molecule having a nucleotide sequence selected from the group consisting of SEQ ID NOs: 1, 3, 5, 7, 9, 11, 13, 15 or complements thereof, that contains DNA markers. DNA markers of the present invention include “dominant” or “codominant” markers. “Codominant markers” reveal the presence of two or more alleles (two per diploid individual) at a locus. “Dominant markers” reveal the presence of only a single allele per locus. The presence of the dominant marker phenotype (e.g., a band of DNA) is an indication that one allele is present in either the homozygous or heterozygous condition. The absence of the dominant marker phenotype (e.g., absence of a DNA band) is merely evidence that “some other” undefined allele is present. In the case of populations where individuals are predominantly homozygous and loci are predominately dimorphic, dominant and codominant markers can be equally valuable. As populations become more heterozygous and multi-allelic, codominant markers often become more informative of the genotype than dominant markers. Examples of DNA markers include restriction fragment length polymorphism (RFLP), random amplified fragment length polymorphism (RAPD), simple sequence repeat polymorphism (SSR), cleavable amplified polymorphic sequences (CAPS), amplified fragment length polymorphism (AFLP), and single nucleotide polymorphism (SNP).

Isolation and identification of nucleic acids encoding SPS or SPS-like polypeptides from cyanobacteria are described in detail in Examples. All or a substantial portion of the nucleic acids of the present invention may be used to isolate cDNAs and nucleic acids encoding homologous polypeptides or fragments thereof from the same or other plant species.

A “substantial portion” of a nucleotide sequence comprises enough of the sequence to afford specific identification and/or isolation of a nucleic acid fragment comprising the sequence. Nucleotide sequences can be evaluated either manually by one skilled in the art, or by using computer-based sequence comparison and identification tools that employ algorithms such as BLAST (Basic Local Alignment Search Tool; Altschul et al., J. Mol. Biol. 215: 403410, 1993; see also www.ncbi.nlm.nih.gov/BLAST/). In general, a sequence of thirty or more contiguous nucleotides is necessary in order to putatively identify a nucleotide sequence as homologous to a gene. Moreover, with respect to nucleotide sequences, gene-specific oligonucleotide probes comprising 30 or more contiguous nucleotides may be used in sequence-dependent methods of gene identification (e.g., Southern hybridization) and isolation (e.g., in situ hybridization of bacterial colonies or bacteriophage plaques). In addition, short oligonucleotides of 12 or more nucleotides may be used as amplification primers in PCR in order to obtain a particular nucleic acid fragment comprising the primers. The skilled artisan, having the benefit of the sequences available as disclosed herein, may now use all or a substantial portion of these disclosed sequences for any purposes known to those skilled in this art. Accordingly, the present invention comprises the complete sequences as reported in the accompanying Sequence Listing, as well as substantial portions of those sequences as defined above.

Isolation of nucleic acids encoding homologous polypeptides using sequence-dependent protocols is well known in the art. Examples of sequence-dependent protocols may include, but may not be limited to, methods of nucleic acid hybridization, and methods of DNA and RNA amplification as exemplified by various uses of nucleic acid amplification technologies (e.g., polymerase chain reaction and ligase chain reaction). For example, structural nucleic acids encoding other SPS or SPS-like transcription factors, either as cDNAs or genomic DNAs, could be isolated directly by using all or a substantial portion of the nucleic acid molecules of the present invention as DNA hybridization probes to screen cDNA or genomic libraries from any desired plant employing methodology well known to those skilled in the art. Methods for forming such libraries are well known in the art. Specific oligonucleotide probes based upon the nucleic acids of the present invention can be designed and synthesized by methods known in the art. Moreover, the entire sequences of the nucleic acids can be used directly to synthesize DNA probes by methods known to the skilled artisan such as random primer DNA labeling, nick translation, or end-labeling techniques, or RNA probes using available in vitro transcription systems. In addition, specific primers can be designed and used to amplify a part or all of the sequences. The resulting amplification products can be labeled directly during amplification reactions or labeled after amplification reactions, and used as probes to isolate full-length cDNAs or genomic DNAs under conditions of appropriate stringency.

Alternatively, the nucleic acids of interest can be amplified from nucleic acid samples using amplification techniques. For instance, the disclosed nucleic acids may be used to define a pair of primers that can be used with the polymerase chain reaction (Mullis et al., Cold Spring Harbor Symp. Quant. Biol. 51:263-273, 1986; EP 50,424; EP 84,796, EP 258,017, EP 237,362; EP 201,184; U.S. Pat. No. 4,683,202; U.S. Pat. No. 4,582,788; and U.S. Pat. No. 4,683,194) to amplify and obtain any desired nucleic acid or fragment directly from mRNA, from cDNA, from genomic libraries or cDNA libraries. PCR and other in vitro amplification methods may also be useful, for example, to clone nucleic acid sequences that code for proteins to be expressed, to make nucleic acids to use as probes for detecting the presence of the desired mRNA in samples, for nucleic acid sequencing, or for other purposes.

In addition, two short segments of the nucleic acids of the present invention may be used in polymerase chain reaction protocols to amplify longer nucleic acids encoding SPS homologous genes from DNA or RNA. For example, the skilled artisan can follow the RACE protocol (Frohman et al., Proc. Natl. Acad. Sci. USA 85: 8998, 1988) to generate cDNAs by using PCR to amplify copies of the region between a single point in the transcript and the 3′ or 5′ end. Primers oriented in the 3′ and 5′ directions can be designed from the nucleic acids of the present invention. Using commercially available 3′RACE or 5′RACE systems (Gibco BRL, Life Technologies, Gaithersburg, Md.), specific 3′ or 5′ cDNA fragments can be isolated (Ohara et al., Proc. Natl. Acad. Sci. USA 86: 5673, 1989; Loh et al., Science 243: 217, 1989). Products generated by the 3′ and 5′ RACE procedures can be combined to generate full-length cDNAs (Frohman and Martin, Techniques 1: 165, 1989).

Nucleic acids of interest may also be synthesized, either completely or in part, especially where it is desirable to provide plant-preferred sequences, by well-known techniques as described in the technical literature. See, e.g., Carruthers et al. (Cold Spring Harbor Symp. Quant. Biol. 47: 411-418, 1982), and Adams et al. (J. Am. Chem. Soc. 105: 661, 1983). Thus, all or a portion of the nucleic acids of the present invention may be synthesized using codons preferred by a selected plant host. Plant-preferred codons may be determined, for example, from the codons used most frequently in the proteins expressed in a particular plant host species. Other modifications of the gene sequences may result in mutants having slightly altered activity.

Availability of the nucleotide sequences encoding SPS or SPS-like proteins facilitates immunological screening of cDNA expression libraries. Synthetic polypeptides representing portions of the amino acid sequences of SPS or SPS-like proteins may be synthesized. These polypeptides can be used to immunize animals to produce polyclonal or monoclonal antibodies with specificity for polypeptides or proteins comprising the amino acid sequences. These antibodies can be then be used to screen cDNA expression libraries to isolate full-length cDNA clones of interest (Lerner, Adv. Immunol. 36: 1, 1984; Sambrook et al., Molecular Cloning: A Laboratory Manual, Cold Spring Harbor Laboratory Press, Cold Spring Harbor, N.Y., 1989). It is understood that people skilled in the art are familiar with the standard resource materials that describe specific conditions and procedures for the construction, manipulation and isolation of antibodies (see, for example, Harlow and Lane, In Antibodies: A Laboratory Manual, Cold Spring Harbor Press, Cold Spring Harbor, N.Y., 1988).

The isolated nucleic acid molecules of the present invention can also be used in antisense technology to suppress endogenous SPS or SPS-like gene expression. To accomplish this, a nucleic acid segment derived from a nucleotide sequence selected from the group consisting of SEQ ID NOs: 1, 3, 5, 7, 9, 11, 13 and 15 is cloned and operably linked to a promoter such that the antisense strand of RNA will be transcribed. The construct is then transformed into plants and the antisense strand of RNA is produced. In plant cells, it has been suggested that antisense RNA inhibit gene expression by preventing the accumulation of mRNA that encodes the enzyme of interest (see, e.g., Sheehy et al., Proc. Nat. Acad. Sci. USA 85: 8805-8809, 1988; and U.S. Pat. No. 4,801,340).

The nucleic acid segment to be introduced generally will be substantially identical to at least a portion of the endogenous SPS or SPS-like gene or genes to be repressed. The sequence, however, needs not to be perfectly identical to inhibit expression. The recombinant vectors of the present invention can be designed such that the inhibitory effect applies to other genes within a family of genes exhibiting homology or substantial homology to the target gene.

For antisense suppression, the introduced sequence also need not be full length relative to either the primary transcription product or fully processed mRNA. Generally, higher homology can be used to compensate for the use of a shorter sequence. Furthermore, the introduced sequence needs not to have the same intron or exon pattern, and homology of non-coding segments may he equally effective. Normally, a sequence from about 30 or 40 nucleotides to about full length nucleotides should be used, though a sequence of at least about 100 nucleotides is preferred, a sequence of at least about 200 nucleotides is more preferred, and a sequence of about 500 to about 1400 nucleotides is most preferred.

The isolated nucleic acid molecules of the present invention can also be used in sense cosuppression to modulate expression of endogenous SPS or SPS-like genes. The suppressive effect may occur where the introduced sequence contains no coding sequence per se, but only intron or untranslated sequences homologous to sequences present in the primary transcript of the endogenous sequence. The introduced sequence generally will be substantially identical to the endogenous sequence to be repressed. This minimal identity will typically be greater than about 65%, but a higher identity might exert a more effective repression of expression of the endogenous sequences. Substantially greater identity of more than about 80% is preferred, though about 95% to absolute identity would be most preferred. As with antisense regulation, the effect should apply to any other proteins within a similar family of genes exhibiting homology or substantial homology.

For sense suppression, the introduced sequence, needing less than absolute identity, also need not be full length, relative to either the primary transcription product or fully processed mRNA. This may be preferred to avoid concurrent production of some plants that are overexpressed. A higher identity in a shorter than the full-length sequence compensates for a longer, less identical sequence. Furthermore, the introduced sequence need not have the same intron or exon pattern, and identity of non-coding segments will be equally effective. Normally, a sequence of the size ranges described above for antisense regulation is used.

Changes in plant phenotypes can be made by specifically inhibiting expression of one or more genes using antisense inhibition or cosuppression technologies (U.S. Pat. Nos. 5,190,931, 5,107,065 and 5,283,323). An antisense or cosuppression construct would act as a dominant negative regulator of gene activity. While conventional mutations can yield negative regulation of gene activity, these effects are most often recessive. The dominant negative regulation available with a transgenic approach may be advantageous from a breeding perspective. In addition, the ability to restrict the expression of specific phenotype to the reproductive tissues of the plant by the use of tissue specific promoters may confer agronomic advantages relative to conventional mutations that may have an effect in all tissues in which a mutant gene is ordinarily expressed.

The person skilled in the art will know that special considerations are associated with the use of antisense or cosuppression technologies in order to reduce expression of particular genes. For example, the proper level of expression of sense or antisense genes may require the use of different chimeric genes utilizing different regulatory elements known to the skilled artisan. Once transgenic plants are obtained by one of the methods described above, it will be necessary to screen individual transgenic plants for those that most effectively display the desired phenotype. Accordingly, the skilled artisan will develop methods for screening large numbers of transformants. The nature of these screens will generally be chosen on practical grounds, and is not an inherent part of the invention. For example, one can screen by looking for changes in gene expression by using antibodies specific for the protein encoded by the gene being suppressed, or one could establish assays that specifically measure enzyme activity. A preferred method will be one that allows large numbers of samples to be processed rapidly, since it will be expected that a large number of transformants will be negative for the desired phenotype.

All or a substantial portion of the nucleic acid fragments of the present invention may also be used as probes for genetically and physically mapping the genes that they are a part of, and as markers for traits linked to, these genes. Such information may be useful in plant breeding in order to develop lines with desired phenotypes. For example, the nucleic acid fragments of the present invention may be used as restriction fragment length polymorphism (RFLP) markers. Southern blots (Maniatis et al., Molecular Cloning: A Laboratory Manual, 2nd ed, Cold Spring Harbor Laboratory, Cold Spring Harbor, N.Y., 1989) of restriction-digested plant genomic DNA may be probed with the nucleic acid fragments of the present invention. The resulting banding patterns may then be subjected to genetic analyses using computer programs such as MapMaker (Lander et al., Genomics 1: 174-181, 1987) in order to construct a genetic map. In addition, the nucleic acid fragments of the present invention may be used to probe Southern blots containing restriction endonuclease-treated genomic DNAs of a set of individuals representing parent and progeny of a defined genetic cross. Segregation of the DNA polymorphisms is noted and used to calculate the position of the nucleotide sequence of the present invention in the genetic map previously obtained using this population (Botstein et al., Am. J. Hum. Genet. 32: 314-331, 1980).

The production and use of plant gene-derived probes for use in genetic mapping is described in Bernatzky and Tanksley (Plant Mol. Biol. Reporter 4: 37-41, 1986). Numerous publications describe genetic mapping of specific cDNA clones using the methodology outlined above or variations thereof. For example, F2 intercross populations, backcross populations, randomly mated populations, near isogenic lines, exotic germplasms, and other sets of individuals may be used for mapping. Such methodologies are well known to those skilled in the art.

Nucleic acid probes derived from the nucleotide sequences of the present invention may also be used for physical mapping (i.e., placement of sequences on physical maps; see Hoheisel et al., In: Nonmammalian Genomic Analysis: A Practical Guide, Academic press, pp. 319-346, 1996, and references cited therein)

In another embodiment, nucleic acid probes derived from the nucleotide sequences of the present invention may be used in direct fluorescence in situ hybridization (FISH) mapping (Trask, Trends Genet. 7: 149-154, 1991). Although current methods of FISH mapping favor use of large clones (several to several hundred KB; see Laan et al., Genome Res. 5: 13-20, 1995), improvements in sensitivity may allow performance of FISH mapping using shorter probes.

A variety of nucleic acid amplification-based methods of genetic and physical mapping may be carried out using the nucleotide sequences of the present invention. Examples include allele-specific amplification (Kazazian et al., J. Lab. Clin. Med. 11:95-96, 1989), polymorphism of PCR-amplified fragments (CAPS; Sheffield et al., Genomics 16:325-332, 1993), allele-specific ligation (Landegren et al., Science 241:1077-1080, 1988), nucleotide extension reactions (Sokolov et al., Nucleic Acid Res. 18:3671, 1990), Radiation Hybrid Mapping (Walter et al., Nat. Genet. 7: 22-28, 1997) and Happy Mapping (Dear and Cook, Nucleic Acid Res. 17: 6795-6807, 1989). For these methods, the sequence of a nucleic acid fragment is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the nucleotide sequence. This, however, is generally not necessary for mapping methods.

The isolated nucleic acid molecules of the present invention may be used in the identification of loss of function mutant phenotypes of a plant, due to a mutation in one or more endogenous genes encoding the SPS or SPS-like polypeptides. This can be accomplished either by using targeted gene disruption protocols or by identifying specific mutants for these genes contained in a population of plants carrying mutations in all possible genes (Ballinger and Benzer, Proc. Natl. Acad Sci USA 86: 9402-9406, 1989; Koes et al., Proc. Natl. Acad. Sci. USA 92: 8149-8153, 1995; Bensen et al., Plant Cell 7: 75-84, 1995). The latter approach may be accomplished in two ways. First, short segments of the nucleic acid fragments of the present invention may be used in polymerase chain reaction protocols in conjunction with a mutation tag sequence primer on DNAs prepared from a population of plants in which mutator transposons or some other mutation-causing DNA element has been introduced. The amplification of a specific DNA fragment with these primers indicates the insertion of the mutation tag element in or near the plant gene encoding SPS or SPS-like polypeptides. Alternatively, the nucleic acid fragments of the present invention may be used as a hybridization probe against PCR amplification products generated from the mutation population using the mutation tag sequence primer in conjunction with an arbitrary genomic site primer, such as that for a restriction enzyme site-anchored synthetic adapter. With either method, a plant containing a mutation in the endogenous gene encoding the SPS or SPS-like polypeptides can be identified and obtained. This mutant plant can then be used to determine or confirm the natural function of the SPS or SPS-like polypeptides disclosed herein.

Methods for introducing genetic mutations into plant genes are well known. For instance, seeds or other plant material can be treated with a mutagenic chemical substance, according to standard techniques. Such chemical substances include, but are not limited to, the following: diethyl sulfate, ethylene imine, ethyl methanesulfonate and N-nitroso-N-ethylurea. Alternatively, ionizing radiation from sources such as, for example, X-rays or gamma rays can be used. Desired mutants are selected by assaying for increased seed mass, oil content and other properties.

Substantially Purified Polypeptides or Proteins

The polypeptides or proteins of the present invention may also include fusion proteins or polypeptides. A protein or fragment thereof that comprises one or more additional polypeptide regions not derived from that protein is a “fusion” protein. Such molecules may be derivatized to contain carbohydrate or other moieties (such as keyhole limpet hemocyanin, etc.). Fusion protein or polypeptide of the present invention is preferably produced via recombinant means.

Nucleic acids that encode all or part of the SPS or SPS-like polypeptides or proteins of the present invention can be expressed, via recombinant means, to yield proteins or polypeptides that can in turn be used to elicit antibodies that are capable of binding the expressed proteins or polypeptides. It may be desirable to derivatize the obtained antibodies, for example with a ligand group (such as biotin) or a detectable marker group (such as a fluorescent group, a radioisotope or an enzyme). Such antibodies may be used in immunoassays for that protein. In a preferred embodiment, such antibodies can be used to screen cDNA expression libraries to isolate full-length cDNA clones of SPS or SPS-like genes (Lerner, Adv. Immunol. 36: 1, 1984; Sambrook et al., Molecular Cloning: A Laboratory Manual, Cold Spring Harbor Laboratory Press, Cold Spring Harbor, N.Y., 1989).

Plant Recombinant DNA Constructs Transformed Plants

The isolated nucleic acids of the present invention can find particular use in creating transgenic plants in which SPS or SPS-like polypeptides are overexpressed. Overexpression of SPS or SPS-like polypeptides in a plant can enhance sucrose synthesis, and thereby lead to improvement in the yield of the plant. It will be particularly desirable to enhance carbohydrates in crop plants. Examples of such crops include soybean, canola, sunflower, and grains such as corn, wheat, rice, rye, and the like.

The term “transgenic plant” refers to a plant that contains an exogenous nucleic acid, which can be derived from the same plant species or from a different species. By “exogenous” it is meant that a nucleic acid originates from outside of the plant into which the nucleic acid is introduced. An exogenous nucleic acid can have a naturally occurring or non-naturally occurring nucleotide sequence. One skilled in the art understands that an exogenous nucleic acid can be a heterologous nucleic acid derived from a different plant species than the plant into which the nucleic acid is introduced or can be a nucleic acid derived from the same plant species as the plant into which it is introduced.

Plant cell, as used herein, includes without limitation, meristematic regions, shoots, leaves, seeds suspension cultures, callus tissue, embryos, roots, gametophytes, sporophytes, pollen and microspores.

The term “genome” as it applies to plant cells encompasses not only chromosomal DNA found within the nucleus, but organelle DNA found within subcellular components of the cell. DNAs of the present invention introduced into plant cells can therefore be either chromosomally integrated or organelle-localized. The term “genome” as it applies to bacteria encompasses both the chromosome and plasmids within a bacterial host cell.

Exogenous nucleic acids may be transferred into a plant cell by the use of a DNA vector or construct designed for such a purpose.

The present invention also relates to a plant recombinant vector or construct comprising a structural nucleotide sequence encoding a SPS or SPS-like protein or polypeptide. Methods that are well known to those skilled in the art may be used to construct the plant recombinant construct or vector of the present invention. These methods include in vitro recombinant DNA techniques, synthetic techniques, and in vivo genetic recombination. Such techniques are described in Sambrook et al. (Molecular Cloning. A Laboratory Manual, Cold Spring Harbor Press, Plainview, N.Y., 1989) and Ausubel et al. (Current Protocols in Molecular Biology, John Wiley & Sons, N.Y., 1989).

A plant recombinant construct or vector of the present invention contains a structural nucleotide sequence encoding a SPS or SPS-like protein or polypeptide of the present invention and operably linked regulatory sequences or control elements. Exemplary regulatory sequences include, but are not limited to, promoters, translation leader sequences, introns and 3′ non-translated sequences. The promoters can be constitutive, inducible, or tissue-specific promoters.

A plant recombinant vector or construct of the present invention will typically comprise a selectable marker that confers a selectable phenotype on plant cells. Selectable markers may also be used to select for plants or plant cells that contain the exogenous nucleic acids encoding polypeptides or proteins of the present invention. The marker may encode biocide resistance, antibiotic resistance (e.g., kanamycin, G418 bleomycin, hygromycin, etc.), or herbicide resistance (e.g., glyphosate, etc.). Examples of selectable markers include, but are not limited to, a neo gene (Potrykus et al., Mol. Gen. Genet. 199: 183-188, 1985) which codes for kanamycin resistance and can be selected for using kanamycin, G418, etc.; a bar gene which codes for bialaphos resistance; a mutant EPSP synthase gene (Hinchee et al., Bio/Technology 6: 915-922, 1988) which encodes glyphosate resistance; a nitrilase gene which confers resistance to bromoxynil (Stalker et al., J. Biol. Chem. 263: 6310-6314, 1988); a mutant acetolactate synthase gene (ALS) which confers imidazolinone or sulphonylurea resistance (EP 154,204); and a methotrexate resistant DHFR gene (Thillet et al., J. Biol. Chem. 263: 12500-12508, 1988).

A plant recombinant vector or construct of the present invention may also include a screenable marker. Screenable markers may be used to monitor expression. Exemplary screenable markers include a β-glucuronidase or uidA gene (GUS) which encodes an enzyme for which various chromogenic substrates are known (Jefferson, Plant Mol. Biol, Rep. 5: 387-405, 1987; Jefferson et al., EMBO J. 6: 3901-3907, 1987); an R-locus gene, which encodes a product that regulates the production of anthocyanin pigments (red color) in plant tissues (Dellaporta et al., Stadler Symposium 11: 263-282, 1988); a β-lactamase gene (Sutcliffe et al., Proc. Natl. Acad. Sci. U.S.A. 75: 3737-3741, 1978), a gene which encodes an enzyme for which various chromogenic substrates are known (e.g., PADAC, a chromogenic cephalosporin); a luciferase gene (Ow et al., Science 234: 856-859, 1986); a xylE gene (Zukowsky et al., Proc. Natl. Acad. Sci. USA 80: 1101-1105, 1983) which encodes a catechol dioxygenase that can convert chromogenic catechols; an α-amylase gene (Ikatu et al., Bio/Technol. 8: 241-242, 1990); a tyrosinase gene (Katz et al., J. Gen. Microbiol. 129: 2703-2714, 1983) which encodes an enzyme capable of oxidizing tyrosine to DOPA and dopaquinone which in turn condenses to melanin; an α-galactosidase, which will turn a chromogenic α-galactose substrate.

Alternatively, the nucleic acid molecules of interest can be amplified from nucleic acid samples using amplification techniques. For instance, the disclosed nucleic acid molecules may be used to define a pair of primers that can be used with the polymerase chain reaction.

In addition, two short segments of the nucleic acid molecules of the present invention may be used in polymerase chain reaction protocols to amplify longer nucleic acid molecules from DNA or cDNA produced from RNA. For example, the skilled artisan can follow the RACE protocol (Frohman et al., Proc. Natl. Acad. Sci. USA 85:8998 (1988) to generate cDNAs by using PCR to amplify copies of the region between a single point in the transcript and the 3′ or 5′ end. Primers oriented in the 3′ and 5′ directions can be designed from the nucleic acid molecules of the present invention. Using commercially available 3′RACE or 5′RACE systems (Gibco BRL, Life Technologies, Gaithersburg, Md. U.S.A.), specific 3′ or 5′ cDNA fragments can be isolated (Ohara et al., Proc. Natl. Acad. Sci. USA 86:5673 (1989); Loh et al., Science 243:217 (1989), both of which are herein incorporated by reference in their entireties). Products generated by the 3′ and 5′ RACE procedures can be combined to generate full-length cDNAs (Frohman and Martin, Techniques 1: 165 (1989).

Another aspect of the present invention relates to methods for obtaining a nucleic acid molecule comprising a nucleotide sequence described herein (i.e. see sequence listing). One method of the present invention for obtaining a nucleic acid molecule encoding all or a substantial portion of the promoter described herein would be: (a) probing a cDNA or genomic library with a hybridization probe comprising a nucleotide sequence encoding all or a substantial portion of a DNA, cDNA, or RNA molecule described herein (b) identifying a DNA clone that hybridizes under stringent conditions to the hybridization probe; (c) isolating the DNA clone identified in step (b); and (d) sequencing the cDNA or genomic fragment that comprises the clone isolated in step (c).

Another method of the present invention for obtaining a nucleic acid molecule described herein: (a) synthesizing a first and a second oligonucleotide primer, wherein the sequences of the first and second oligonucleotide primer encode two different portions of the nucleotide sequence described herein, and are manufactured in such a way as to allow DNA amplification (for example, PCR®) (Maniatis et al., Molecular Cloning: A Laboratory Manual, Second Edition (1989) Cold Spring Harbor Laboratory, Cold Spring Harbor, N.Y.,; Hartl, et al., Genetics, Analysis of genes and genomes, 5^{th} edition, Jones and Bartlett Publishers, Inc., Sudbury, Mass.); and (b) amplifying and obtaining the nucleic acid molecule directly from genomic libraries using the first and second oligonucleotide primers of step (a) wherein the nucleic acid molecule encodes all or a substantial portion of the sequence described herein.

All or a substantial portion of the nucleic acid molecules of the present invention may also be used as probes for genetically and physically mapping the genes that they are a part of, and as markers for traits linked to those genes. Such information may be useful in plant breeding in order to develop lines with desired phenotypes. For example, the nucleic acid molecules of the present invention may be used as restriction fragment length polymorphism (RFLP) markers. Southern blots (Maniatis et al., Molecular Cloning: A Laboratory Manual, Second Edition (1989) Cold Spring Harbor Laboratory, Cold Spring Harbor, N.Y.) of restriction-digested plant genomic DNA may be probed with the nucleic acid fragments of the present invention. The resulting banding patterns may then be subjected to genetic analyses using computer programs such as MapMaker (Lander et al., Genomics 1:174-181 (1981), or can be analyzed by one skilled in the art, in order to construct a genetic map. Fragments of the present invention may be used to probe Southern blots containing restriction endonuclease-treated genomic DNAs of a set of individuals representing parent and progeny of a defined genetic cross. Segregation of the DNA polymorphisms is noted and used to calculate the position of the nucleotide sequence of the present invention in the genetic map previously obtained using this population (Botstein et al., Am. J. Hum. Genet. 32:314-331 (1980).

Methods for determining gene expression, even expression of a gene from an introduced transgene are common in the art, and include RT-PCR, Northern blots, and Taqman®. Taqman® (PE Applied Biosystems, Foster City, Calif.) is described as a method of detecting and quantifying the presence of a DNA or RNA/cDNA molecule and is fully described in the instructions provided by the manufacturer, and at their website. Briefly, in the case of a genomic sequence a FRET oligonucleotide probe is designed which overlaps the genomic flanking and insert DNA junction. The FRET probe and PCR primers (one primer in the insert DNA sequence and one in the flanking genomic sequence) are cycled in the presence of a thermostable polymerase and dNTPs. Hybridization of the FRET probe results in cleavage and release of the fluorescent moiety away from the quenching moiety on the FRET probe. A fluorescent signal indicates the presence of the flanking/transgene insert DNA due to successful amplification and hybridization.

Included within the terms “selectable or screenable marker genes” are also genes that encode a secretable marker whose secretion can be detected as a means of identifying or selecting for transformed cells. Examples include markers that encode a secretable antigen that can be identified by antibody interaction, or even secretable enzymes that can be detected catalytically. Secretable proteins fall into a number of classes, including small, diffusible proteins detectable, e.g., by ELISA, small active enzymes detectable in extracellular solution (e.g., α-amylase, β-lactamase, phosphinothricin transferase), or proteins which are inserted or trapped in the cell wall (such as proteins which include a leader sequence such as that found in the expression unit of extension or tobacco PR-S). Other possible selectable and/or screenable marker genes will be apparent to those of skill in the art.

In addition to a selectable marker, it may be desirous to use a reporter gene. In some instances a reporter gene may be used with or without a selectable marker. Reporter genes are genes that are typically not present in the recipient organism or tissue and typically encode for proteins resulting in some phenotypic change or enzymatic property. Examples of such genes are provided in K. Wising et al. (Ann. Rev. Genetics 22: 421, 1988). Preferred reporter genes include the beta-glucuronidase (GUS) of the uidA locus of E. coli, the chloramphenicol acetyl transferase gene from Tn9 of E. coli, the green fluorescent protein from the bioluminescent jellyfish Aequorea victoria, and the luciferase genes from firefly Photinus pyralis. An assay for detecting reporter gene expression may then be performed at a suitable time after said gene has been introduced into recipient cells. A preferred such assay entails the use of the gene encoding beta-glucuronidase (GUS) of the uidA locus of E. coli as described by Jefferson et al. (Biochem. Soc. Trans. 15: 17-19, 1987) to identify transformed cells.

In preparing the recombinant DNA constructs of the present invention, the various components of the construct or fragments thereof will normally be inserted into a convenient cloning vector, e.g., a plasmid that is capable of replication in a bacterial host, e.g., E. coli. Numerous vectors exist that have been described in the literature, many of which are commercially available. After each cloning, the cloning vector with the desired insert may be isolated and subjected to further manipulation, such as restriction digestion, insertion of new fragments or nucleotides, ligation, deletion, mutation, resection, etc. so as to tailor the components of the desired sequence. Once the construct has been completed, it may then be transferred to an appropriate vector for further manipulation in accordance with the manner of transformation of the host cell.

A plant recombinant vector or construct of the present invention may also include a chloroplast transit peptide, in order to target the polypeptide or protein of the present invention to the plastid. The term “plastid” refers to the class of plant cell organelles that includes amyloplasts, chloroplasts, chromoplasts, elaioplasts, eoplasts, etioplasts, leucoplasts, and proplastids. These organelles are self-replicating, and contain what is commonly referred to as the “chloroplast genome,” a circular DNA molecule that ranges in size from about 120 to about 217 kb, depending upon the plant species, and which usually contains an inverted repeat region. Many plastid-localized proteins are expressed from nuclear genes as precursors and are targeted to the plastid by a chloroplast transit peptide (CTP), which is removed during the import steps. Examples of such chloroplast proteins include the small subunit of ribulose-1,5-biphosphate carboxylase (ssRUBISCO, SSU), 5-enolpyruvateshikimate-3-phosphate synthase (EPSPS), ferredoxin, ferredoxin oxidoreductase, the light-harvesting-complex protein I and protein II, and thioredoxin F. It has been demonstrated that non-plastid proteins may be targeted to the chloroplast by use of protein fusions with a CTP and that a CTP sequence is sufficient to target a protein to the plastid. Those skilled in the art will also recognize that various other chimeric constructs can be made that utilize the functionality of a particular plastid transit peptide to import the enzyme into the plant cell plastid depending on the promoter tissue specificity.

Transgenic plants of the present invention preferably have incorporated into their genome or transformed into their chloroplast or plastid genomes a selected polynucleotide (or “transgene”), that comprises at least a structural nucleotide sequence that encodes a SPS or SPS-like polypeptide whose amino acid sequence disclosed herein. Transgenic plants are also meant to comprise progeny (descendant, offspring, etc.) of any generation of such a transgenic plant; a fertile plant. A seed of any generation of all such transgenic plants wherein said seed comprises a DNA sequence encoding the SPS or SPS-like polypeptide of the present invention is also an important aspect of the invention.

In one embodiment, the transgenic plants of present invention will have enhanced sucrose synthesis due to the overexpression of an exogenous nucleic acid encoding a SPS or SPS-like polypeptide as disclosed and described herein. In a preferred embodiment, the transgenic plants of present invention will have increased number and/or size of seeds, fruits, roots, and tubers. In a more preferred embodiment, the transgenic plants of present invention will have increased yield.

The term “increased size”, as used herein in reference to an organ (e.g., seed) of the transgenic plant of the present invention, means that the organ (e.g., seed) has a significantly greater volume or dry weight or both as compared to the volume or dry weight of same organ of a corresponding wild type plant. It is recognized that there can be natural variation in the size of an organ (e.g., seed) of a particular plant species. However, the organ (e.g., seed) of increased size of the transgenic plant of the present invention readily can be identified by sampling a population of that organ (e.g., seed) and determining that the normal distribution of the organ (e.g., seed) sizes is greater, on average, than the normal distribution of the organ (e.g., seed) sizes of a wild type plant. The volume or dry weight of an organ (e.g., seed) is, on average, usually at least 10% greater, 30% greater, 50% greater, 75% greater, more usually at least 100% greater, and most usually at least 200% greater than in the corresponding wild type plant species.

The DNA constructs of the present invention may be introduced into the genome of a desired plant host by a variety of conventional transformation techniques, which are well known to those skilled in the art. Preferred methods of transformation of plant cells or tissues are the Agrobacterium mediated transformation method and the biolistics or particle-gun mediated transformation method. Suitable plant transformation vectors for the purpose of Agrobacterium mediated transformation include those derived from a Ti plasmid of Agrobacterium tumefaciens, as well as those disclosed, e.g., by Herrera-Estrella et al. (Nature 303: 209, 1983); Bevan (Nucleic Acids Res. 12: 8711-8721, 1984); Klee et al. (Bio-Technology 3 (7): 637-642, 1985); EP120,516. In addition to plant transformation vectors derived from the Ti or root-inducing (Ri) plasmids of Agrobacterium, alternative methods can be used to insert the DNA constructs of this invention into plant cells. Such methods may involve, but are not limited to, for example, the use of liposomes, electroporation, chemicals that increase free DNA uptake, free DNA delivery via microprojectile bombardment, and transformation using viruses or pollen.

A plasmid expression vector suitable for the introduction of a nucleic acid encoding a SPS or SPS-like polypeptide in monocots using electroporation or particle-gun mediated transformation is composed of the following: a promoter that is constitutive or tissue-specific; an intron that provides a splice site to facilitate expression of the gene, such as the Hsp70 intron (PCT Publication WO93/19189); and a 3′ polyadenylation sequence such as the nopaline synthase 3′ sequence (NOS 3′; Fraley et al., Proc. Natl. Acad. Sci. USA 80: 4803-4807, 1983). This expression cassette may be assembled on high copy replicons suitable for the production of large quantities of DNA.

An example of a useful Ti plasmid cassette vector for plant transformation is pMON-17227%. This vector is described in PCT Publication WO 92/04449, and contains a gene encoding an enzyme conferring glyphosate resistance (denominated CP4), which is useful as a selection marker gene for many plants. The gene is fused to the Arabidopsis EPSPS chloroplast transit peptide (CTP2) and expressed from the FMV promoter as described therein.

When adequate numbers of cells (or protoplasts) containing the exogenous nucleic acid encoding a SPS or SPS-like polypeptide are obtained, the cells (or protoplasts) can be cultured to regenerate into whole plants. Such regeneration techniques rely on manipulation of certain phytohormones in a tissue culture growth medium, typically relying on a biocide and/or herbicide marker that has been introduced together with the desired nucleotide sequences. Choice of methodology for the regeneration step is not critical, with suitable protocols being available for hosts from Leguminosae (soybean, clover, etc.), Umbelliferae (carrot), Cruciferae (radish, canola/rapeseed, etc.), Cucurbitaceae (melons and cucumber), Gramineae (wheat, barley, rice, maize, etc.), Solanaceae (potato, tobacco, tomato, peppers), various floral crops, such as sunflower, and nut-bearing trees, such as almonds, cashews, walnuts, and pecans. See, for example, Ammirato et al. (Handbook of Plant Cell Culture—Crop Species, Macmillan Publ. Co., 1984), Shimamoto et al. (Nature 338: 274-276, 1989); Fromm (UCLA Symposium on Molecular Strategies for Crop Improvement, Keystone, Colo., 1990), Vasil et al. (Bio/Technology 8: 429-434, 1990), Vasil et al. (Bio/Technology 10: 667-674, 1992), Hayashimoto (Plant Physiol. 93: 857-863, 1990), and Datta et al. (Bio/technology 8: 736-740, 1990). Plant regeneration from cultured protoplasts is described in Evans et al. (Protoplasts Isolation and Culture, Handbook of Plant Cell Culture, pp. 124-176, MacMillilan Publishing Company, N.Y., 1983) and Binding (Regeneration of Plants-Plant Protoplasts, pp. 21-73, CRC Press, Boca Raton, 1985). Regeneration can also be obtained from plant callus, explants, organs, or parts thereof. Such regeneration techniques are described generally in Klee et al. (Ann. Rev. Plant Phys. 38: 467-486, 1987).

A transgenic plant formed using Agrobacterium transformation methods typically contains a single exogenous gene on one chromosome. Such transgenic plants can be referred to as being heterozygous for the added exogenous gene. More preferred is a transgenic plant that is homozygous for the added exogenous gene; i.e., a transgenic plant that contains two added exogenous genes, one gene at the same locus on each chromosome of a chromosome pair. A homozygous transgenic plant can be obtained by sexually mating (selfing) an independent segregant transgenic plant that contains a single exogenous gene, germinating some of the seed produced and analyzing the resulting plants produced for the exogenous gene of interest.

The development or regeneration of transgenic plants containing the exogenous nucleic acid that encodes a polypeptide or protein of interest is well known in the art. Preferably, the regenerated plants are self-pollinated to provide homozygous transgenic plants, as discussed above. Otherwise, pollen obtained from the regenerated plants is crossed to seed-grown plants of agronomically important lines. Conversely, pollen from plants of these important lines is used to pollinate regenerated plants. A transgenic plant of the present invention containing a desired SPS or SPS-like polypeptide is cultivated using methods well known to one skilled in the art.

Plants that can be made to have increased sucrose synthesis and export from their source tissues (e.g., leaves) by practice of the present invention include, but are not limited to, apple, apricot, artichoke, avocado, banana, barley, beans, beet, blackberry, blueberry, canola, cantaloupe, carrot, cherry, citrus, clementines, coffee, corn, cotton, cucumber, eggplant, figs, grape, grapefruit, honey dew, kiwifruit, lettuce, leeks, lemon, lime, mango, melon, nut, oat, orange, papaya, parsley, pea, peach, peanut, pear, pepper, persimmon, pineapple, plum, potato, pumpkin, radish, raspberry, rice, rye, sorghum, soybean, squash, strawberry, sugarbeet, sugarcane, sunflower, sweet potato, sweetgum, tangerine, tobacco, tomato, a vine, watermelon, wheat, yams, and zucchini.

The present invention also further provides method for generating a transgenic plant having increased sucrose synthesis and export from source tissues (e.g., leaves), the method comprising the steps of: a) introducing into the genome of the plant an exogenous nucleic acid, wherein the exogenous nucleic acid comprises in the 5′ to 3′ direction i) a promoter that functions in the cells of the plant, the promoter operably linked to; ii) a structural nucleic acid sequence encoding an SPS polypeptide the amino acid sequence of which is substantially identical to a member selected from the group consisting of any SPS or portion thereof, present in the sequence listing, the structural nucleic acid sequence operably linked to; iii) a 3′ non-translated nucleic acid sequence that functions in the cells of the plant to cause transcriptional termination; b) obtaining transformed plant cells containing the nucleic acid sequence of step (a); and c) regenerating from the transformed plant cells a transformed plant in which the SPS polypeptide is expressed.

Many agronomic traits can affect “yield”. For example, these could include, without limitation, plant height, pod number, pod position on the plant, number of internodes, incidence of pod shatter, grain size, efficiency of nodulation and nitrogen fixation, efficiency of nutrient assimilation, resistance to biotic and abiotic stress, carbon assimilation, plant architecture, resistance to lodging, percent seed germination, seedling vigor, and juvenile traits. For example, these could also include, without limitation, efficiency of germination (including germination in stressed conditions), growth rate (including growth rate in stressed conditions), ear number, seed number per ear, seed size, composition of seed (starch, oil, protein), characteristics of seed fill. “Yield” can be measured in may ways, these might include test weight, seed weight, seed number per plant, seed weight, seed number per unit area (i.e. seeds, or weight of seeds, per acre), bushels per acre, tonnes per acre, tons per acre, kilo per hectare. In an embodiment, a plant of the present invention might exhibit an enhanced trait that is a component of yield.

“Promoter” refers to a DNA sequence that binds an RNA polymerase (and often other transcription factors as well) and promotes transcription of a downstream DNA sequence. Said sequence can be an RNA that has function, such as rRNA (ribosomal RNA), RNAi, dsRNA, or tRNA (transfer RNA). Often, the RNA produced is a hetero-nuclear (hn) RNA that has introns which are spliced out to produce an mRNA (messenger RNA). A “plant promoter” is a native or non-native promoter that is functional in plant cells.

Promoters are typically comprised of multiple distinct “cis-acting transcriptional regulatory elements,” or simply “cis-elements,” each of which can confer a different aspect of the overall control of gene expression (Strittmatter and Chua, Proc. Nat. Acad. Sci. USA 84:8986-8990, 1987; Ellis et al., EMBO J. 6:11-16, 1987; Benfey et al., EMBO J. 9:1677-1684, 1990). “cis elements” bind trans-acting protein factors that regulate transcription. Some cis elements bind more than one factor, and trans-acting transcription factors may interact with different affinities with more than one cis element (Johnson and McKnight, Ann. Rev. Biochem. 58:799-839, 1989). Plant transcription factors, corresponding cis elements, and analysis of their interaction are discussed, for example, in: Martin, Curr. Opinions Biotech. 7:130-138, 1996; Murai, In: Methods in Plant Biochemistry and Molecular Biology, Dashek, ed., CRC Press, 1997, pp. 397-422; and Methods in Plant Molecular Biology, Maliga et al., eds., Cold Spring Harbor Press, 1995, pp. 233-300. The promoter sequences of the present invention can contain “cis elements” which can modulate gene expression. Cis elements can be part of the promoter, or can be upstream or downstream of said promoter. Cis elements (or groups thereof) acting at a distance from a promoter are often referred to as repressors or enhancers. Enhancers act to upregulate the transcriptional initiation rate of RNA polymerase at a promoter, repressors act to decrease said rate. In some cases the same elements can be found in a promoter and an enhancer or repressor.

Cis elements can be identified by a number of techniques, including deletion analysis, i.e., deleting one or more nucleotides from the 5′ end or internal to a promoter; DNA binding protein analysis using Dnase I footprinting, methylation interference, electrophoresis mobility-shift assays (EMSA or gel shift assay), in vivo genomic footprinting by ligation-mediated PCR, and other conventional assays; or by sequence similarity with known cis element motifs by conventional sequence comparison methods. The fine structure of a cis element can be further studied by mutagenesis (or substitution) of one or more nucleotides or by other conventional methods. See, e.g., Methods in Plant Biochemistry and Molecular Biology, Dashek, ed., CRC Press, 1997, pp. 397-422; and Methods in Plant Molecular Biology, Maliga et al., eds., Cold Spring Harbor Press, 1995, pp. 233-300.

Cis elements can be obtained by chemical synthesis or by cloning from promoters that includes such elements, and they can be synthesized with additional flanking sequences that contain useful restriction enzyme sites to facilitate subsequence manipulation. In one embodiment, the promoters are comprised of multiple distinct “cis-acting transcriptional regulatory elements,” or simply “cis-elements,” each of which can modulate a different aspect of the overall control of gene expression (Strittmatter and Chua, Proc. Nat. Acad. Sci. USA 84:8986-8990, 1987; Ellis et al., EMBO J. 6:11-16, 1987; Benfey et al., EMBO J. 9:1677-1684, 1990). For example, combinations of cis element regions or fragments of the 35S promoter can show tissue-specific patterns of expression (see U.S. Pat. No. 5,097,025). In one embodiment sequence regions comprising “cis elements” or “cis elements” of the nucleic acid sequences of SEQ ID NO: 1 can be identified using computer programs designed specifically to identity cis elements, domains, or motifs within sequences by a comparison with known cis elements or can be used to align multiple 5′ regulatory sequences to identify novel cis elements. Activity of a cloned promoter or putative promoter (cloned or produced in any number of ways including but not limited to; isolation form an endogenous piece of genomic DNA directly cloning or by PCR; chemically synthesizing the piece of DNA) can be tested in any number of ways including testing for RNA (Northern, Taqman®, quantitative PCR, etc.) or production of a protein with an activity that is testable (i.e. GUS, chlorempenicaol acetyl transferase (CAT)). Multimerization of elements or partial or complete promoters can change promoter activity (i.e. e35S, U.S. Pat. Nos. 5,359,142, 5,196,525, 5,322,938, 5,164,316, and 5,424,200, and below). Cis elements may work by themselves or in concert with other elements of the same or different type, i.e. hormone- or light-responsive elements.

The technological advances of high-throughput sequencing and bioinformatics have provided additional molecular tools for promoter discovery. Particular target plant cells, tissues, or organs at a specific stage of development, or under particular chemical, environmental, or physiological conditions can be used as source material to isolate the mRNA and construct cDNA libraries. The cDNA libraries are quickly sequenced and the expressed sequences catalogued electronically. Using sequence analysis software, thousands of sequences can be analyzed in a short period, and sequences from selected cDNA libraries can be compared. The combination of laboratory and computer-based subtraction methods allows researchers to scan and compare cDNA libraries and identify sequences with a desired expression profile. For example, sequences expressed preferentially in one tissue can be identified by comparing a cDNA library from one tissue to cDNA libraries of other tissues and electronically “subtracting” common sequences to find sequences only expressed in the target tissue of interest. The tissue enhanced sequence can then be used as a probe or primer to clone the corresponding full-length cDNA. A genomic library of the target plant can then be used to isolate the corresponding gene and the associated regulatory elements, including promoter sequences.

The term “tissue-specific promoter” means a regulatory sequence that causes an enhancement of transcription from a downstream gene in specific cells or tissues at specific times during plant development, such as in vegetative tissues or reproductive tissues. Examples of tissue-specific promoters under developmental control include promoters that initiate transcription only (or primarily only) in certain tissues, such as vegetative tissues, e.g., roots, leaves or stems, or reproductive tissues, such as fruit, ovules, seeds, pollen, pistols, flowers, or any embryonic tissue. Reproductive tissue specific promoters may be, e.g., ovule-specific, embryo-specific, endosperm-specific, integument-specific, seed coat-specific, pollen-specific, petal-specific, sepal-specific, or some combination thereof. One skilled in the art will recognize that a tissue-specific promoter may drive expression of operably linked sequences in tissues other than the target tissue. Thus, as used herein a tissue-specific promoter is one that drives expression preferentially in the target tissue, but may also lead to some expression in other tissues as well. Thus tissue specific and tissue enhanced can be used almost interchangeably, as one who is skilled in the art knows that tissue specific expression is rare.

The invention also shows that specific expression of an SPS in the mesophyll tissue of a C4 plant is particularly advantageous for enhancement of source in the plant and any promoter specifically active in the mesophyll cells of vegetative tissues, such as leaves and stems can be used. For example, the PPDK promoter from maize (Matsuoka et al, PNAS (USA) 90:9586-9590 (1993)) may be advantageously used as well as the promoter from the small subunit of rubisco from a C4 plant (Nomura et al, Plant Mol Biol 44:99-106). Other mesophyll specific promoters from other plants such as maize, wheat, barley and rice may also be obtained and used in connection with the present invention as well as other heterologous promoters from other sources that are shown to function in a mesophyll-specific manner.

All publications and patents mentioned in this specification are herein incorporated by reference as if each individual publication or patent was specially and individually stated to be incorporated by reference.

The following examples are provided to better elucidate the practice of the present invention and should not be interpreted in any was to limit the scope of the present invention. Those skilled in the art will recognize that various modifications, truncations, etc., can be made to the methods and genes described herein while not departing from the spirit and scope of the present invention. Those skilled in the art will also recognize there exist numerous equivalents to the specific embodiments described herein. Such equivalents are intended also to be within the scope of the present invention and claims.

### EXAMPLES

### Example 1

### Reagents and Materials

General biochemicals, buffers and GeneElute Spin columns were purchased from Sigma (St. Louis, Mo.). PCR clean up and Miniprep kits were from Qiagen (Valencia, Calif.). ¹⁴C labeled UDP-glc was purchased from Amersham (Piscataway, N.J.). Anabaena gDNA was purchased from Dr. Teresa Thiel at University of Missouri in St. Louis. The rapid ligation kit was purchased from Roche (Indianapolis, Ind.). All restriction enzymes and T4 ligase were from New England Biolabs (Beverly, Mass.). Platinum Taq Hi Fidelity polymerase, DH5α cells, DH10β and all oligonucleotide primers were purchased from Life Technologies (Gibco BRL, Rockville, Md.).

### Example 2

### tBlastn Search of Databases

A tBlastn (Altschul, et al., J. Mol. Biol. 215: 403-410, 1990; Altschul, et al., Nucleic Acids Res. 25: 3389-3402, 1997) search of the Anabaena database (available at Cyanobase) was performed utilizing the publicly available Synechocystis sequence for SPS (gi1001295). A second tBlastn search was then performed on the same data set using the Maize SSII (gi1351136) sequence. This second search was performed to eliminate any ambiguity in the hits of the first set since SPS and sucrose synthase (SS) are somewhat similar in domains along their primary sequences. The top hits from these searches that were not SS genes and had similarity to the SPS gene were cloned and overexpressed in E. coli. From the identities alone it was not directly obvious that these sequences were SPS genes. Activity assays were performed in order to unequivocally assign SPS function to these proteins.

The same data mining process (tBlastn as described above) and selection were also used to mine other data bases containing Nostoc punctiforme, Marine Synechococcus, and Prochlorococcus marinus DNA (all available at JGI Microbial Genomes project in public). This has resulted in the identification of and first annotation of putative SPS genes from these respective genomes.

### Example 3

### Protein Alignments

Protein alignment trees were created with Clustal X 1.8 (Thompson et. al., Nucleic Acids Research 24: 4876-4882, 1997). Sequences of the present invention and those from Synechocystis and higher plants including maize, rice, tomato, potato, sugarcane, sugarbeet, spinach and Arabidopsis thaliana were first aligned under default conditions for a complete alignment and adjustments made if necessary. These sequence alignments were then used to produce a Neighbor joining bootstrap protein tree in the same application using default parameters with exception of the use of 1000 iterations. The phylogenetic tree (not shown) grouped all Nostoc and Anabaena contigs on one large clade and Synnechococcus, Prochlorococcus and Synechocystis on the other next to the large clade. The tree also grouped all higher plant SPS protens together on a separate clade, suggesting differences between the cyanobacterial and higher plant SPS proteins.

### Example 4

### Sequence Isolation from Anabaena

Since these genes were identified as part of contiguous gDNA and were not previously annotated as such, the coding sequences (cDNA) were identified (ORF search based on blast results) and excised. Primers used were made to the coding sequences as they were found in the contigs identified with the exceptions as described. Primers for Anabaena SPS sequences are listed in Table 4. Additional primers for sequencing out of pET-28b(+) were T7 promoter and reverse primers (Novagen, WI). They are plasmid specific.

TABLE 5

Primers for Anabaena SPS genes.
Table 5a. PCR primers for insertion into
pTrcHis and pET 28b(+) NcoI

| | |
|---|---|
| Names/position | SEQ ID NOs |

| | | |
|---|---|---|
| 5′ primers | | |
| AGATCTCCATGGCCCAAAATAA | C154F Start | SEQ ID No: 15 |
| AAAACATCG | | |
| AGATCTCCATGGCCTCTAACAC | C287F Start | SEQ ID No: 17 |
| TGAAAAACG | | |
| 3′ primers (5′ to 3′) | | |
| GCGAATTCTCGAG CTA CGC | C154R Stop | SEQ ID No: 16 |
| TGC AAC AGC CTC | | |
| GCGAATTCTCGAG CTA TTT AGT | C287R Stop | SEQ ID No: 18 |
| TAC CAA TGC TGG | | |

TABLE 5b
3′ primers for removal of stop and
insertion into pMON23450 with Flag. Also for
insertion into pET-28 b(+) with C-terminal Histag.

| | | |
|---|---|---|
| CGA GGA ATT CGC TGC AAC AGC | C154R3 (Stop) | SEQ ID No: 19 |
| CTC TTT TTC | | |
| GCT CGA ATT CGC TTT AGT TAC | C287R2 (Stop) | SEQ ID No: 20 |
| CAA TGC TGG C | | |

TABLE 5c
Sequencing Primers.

| | | |
|---|---|---|
| GATCACGTATTTGATTATTTACCGG | C154SQ1 | SEQ ID No: 21 |
| | (bp253F) | |
| CCG GTA AAT AAT CAA ATA CGT | C154SQ2 | SEQ ID No: 22 |
| GAT C | (bp253R) | |
| CGGAAACATTGAAAAGTCGG | C154SQ3 | SEQ ID No: 23 |
| | (bp618F) | |
| CCG ACT TTT CAA TGT TTC CG | C154SQ4 | SEQ ID No: 24 |
| | (bp618R) | |
| GCG ATG GCT AGC AAA ACT CC | C154SQ5 | SEQ ID No: 25 |
| | (bp982F) | |
| GGA GTT TTG CTA GCC ATC GC | C154SQ6 | SEQ ID No: 26 |
| | (bp982R) | |
| GTTAATTACCCATTAGTGCATAC | C287SQ1 | SEQ ID No: 27 |
| | (bp319F) | |
| GTA TGC ACT AAT GGG TAA | C287SQ2 | SEQ ID No: 28 |
| TTA AC | (bp319R) | |
| GTG GTC TTG TAT GTA GGA CGC | C28SQ3 | SEQ ID No: 29 |
| | (bp676F) | |
| GCG TCC TAC ATA CAA GAC CAC | C287SQ4 | SEQ ID No: 30 |
| | (bp676R) | |
| GCA ATG GCA AGT GGT ACA C | C287SQ5 | SEQ ID No: 31 |
| | (bp982F) | |
| GTG TAC CAC TTG CCA TTG C | C287SQ6 | SEQ ID No: 32 |
| | (bp982R) | |

Notes:
All primers are from 5′ to 3′. Note for all second codons are changed to ALA to insert Nco I site. 3′ primers can use EcoRI or Xho I as needed for constructs. F: forward, R: reverse.

Sequence Identification

Comparison of SPS sequences alone did not unambiguously identify these cyanobacterial SPS genes disclosed in the present invention. The uniqueness of these genes in the SPS family is highlighted by the overall identity of these genes to Maize SPS I and Synechocystis SPS as shown in Table 1. In these particular species of cyanobacteria the SPS genes share greater identity to the sucrose synthase (SS) genes of Synechocystis and plants. As such these cyanobacterial SPS sequences were identified based upon selection (tblastn) first by SPS and then by SS. Top hits from this selection that were not SS genes showed very low identities when compared with the publicly available SPS sequences from maize and Synechocystis. These genes were further examined for conserved motifs containing putative essential histidine residues (Sinha et al., Biochim Biophys Acta 1388: 397-404, 1998). Those that contained the essential histidine residues became putative SPS genes and were selected for activity analysis.

### Example 5

### PCR Cloning of Anabaena SPS Genes

All molecular biology analyses were performed using standard protocols unless otherwise noted (Ausubel et al., Current Protocols in Molecular Biology, John Wiley & Sons, New York, N.Y., 2000; Sambrook et al., Molecular Cloning, A Laboratory Manual, Cold Spring Harbor Press, Plainview, N.Y., 1989). Anabaena Genomic DNA was utilized as template in standard PCR reactions. These reactions contained template (10-200 ng), primers (50 to 150 pmol), 1.5 to 3.0 mM MgCl₂ and 0.5 to 1 U polymerase (Platinum Taq Hi Fidelity with its buffer as supplied), in a total sample volume of 50 uL. Thermal cycling conditions consisted of denaturation at 94° C. for 15 sec followed by annealing at 52° C. for 25 sec and extension at 68° C. for 1.3 min for 30 cycles. Samples were then held at 4-10° C. until use.

PCR products under conditions described above were produced using Anabaena DNA as template. The C154 product was made using C154F (SEQ ID NO: 15) and C154R (SEQ ID NO: 16) primers. The C287 product was produced using C287F (SEQ ID NO: 17) and C287R (SEQ ID NO: 18). Both primer sets had Nco I sites incorporated into their 5′ regions (requiring a change of the second codon in both instances, i.e., TTC (Phe) to GCC (Ala) C154 and AAC (Asn) to GCC (Ala) C287) and XhoI incorporated into their 3′ region. Products from these reactions were analyzed by analytical agarose gel electrophoresis (1.0% TAE). Products were then purified using Qiagen PCR clean up kits following the manufacturer's protocol. Recovered products and pET-28b were digested with NcoI and XhoI and were purified by gel electrophoresis (0.7-1.0% TAE). Digested pET-28b was treated with calf intestinal phosphatase (CIP) prior to gel purification. Gel-purified, digested vector and PCR products from the excised bands were then recovered using the Gene Elute columns (Sigma, St. Louis, Mo.) following the manufacture's protocol. The recovered purified vectors and fragments were then ligated using T4 Ligase (NEB) under standard conditions or by utilizing the rapid ligation kit (Roche) following manufacturer's protocol. The resultant ligation mixture was electroporated or chemically transformed using the manufacture's protocol (Gibco, BRL) into DH5α or DH10β cells for propagation of the DNA. The transformed cells were plated on the appropriate antibiotic (e.g. pET-28, Kan) and resultant colonies containing the insert of interest were selected by colony PCR using the same PCR primers that were used to produce original product. Plasmids were further confirmed by miniprep and specific restriction digestion to remove the inserted fragment. Plasmids were then sent for fully automated cycle sequencing (GSC, standard protocols both stands) using sequencing primers as described in Table 4 above.

Vectors that had a confirmed insert by sequencing were digested with (NcoI/XhoI) in order to excise the gene of interest for subcloning into pMON 23450, a binary plant expression vector, also digested with NcoI/XhoI and treated with CIP. The resulting fragment and vector were ligated (standard protocols) as above to ultimately produce a binary vector containing the gene of interest. Inserts were again confirmed by plasmid isolation and digestion.

C-Terminal Flag and His Tagged vectors

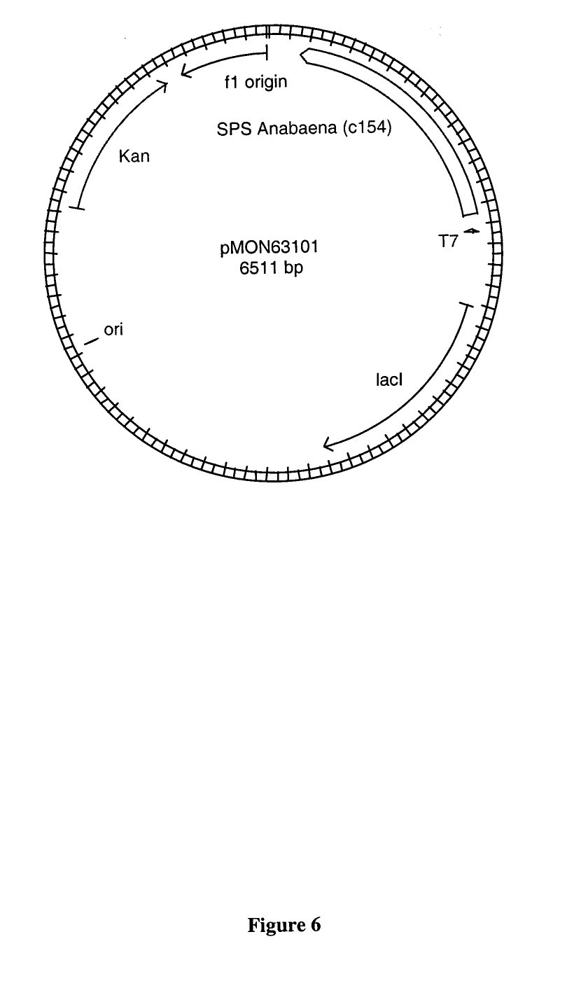

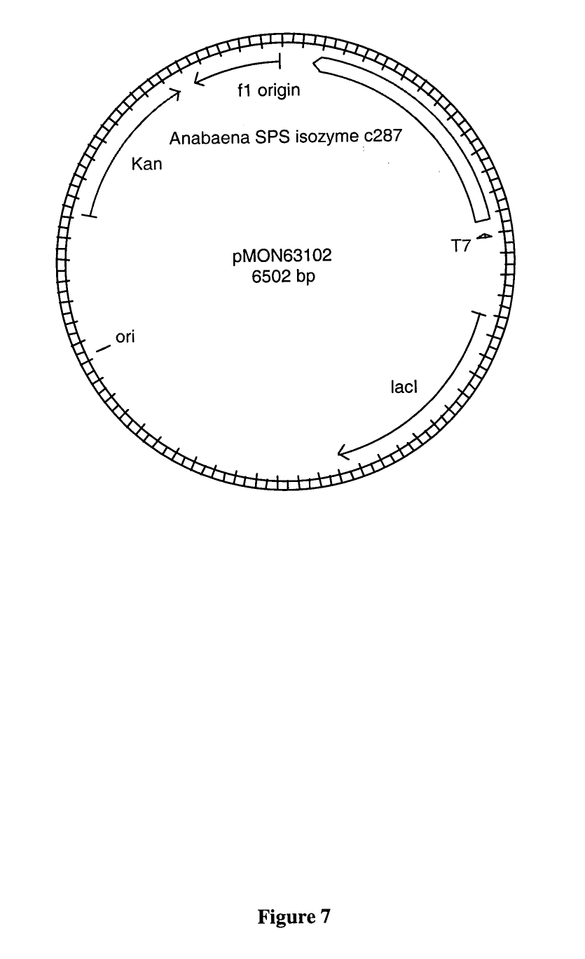

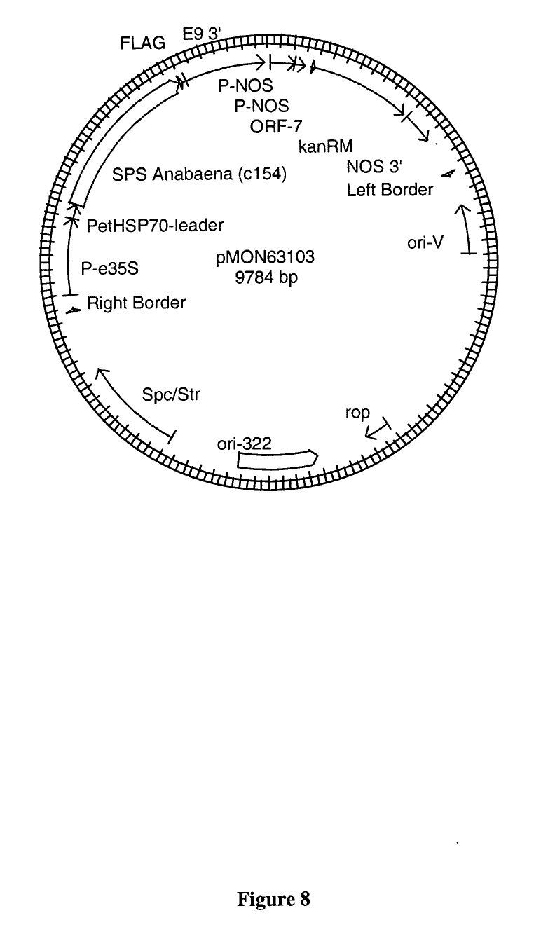

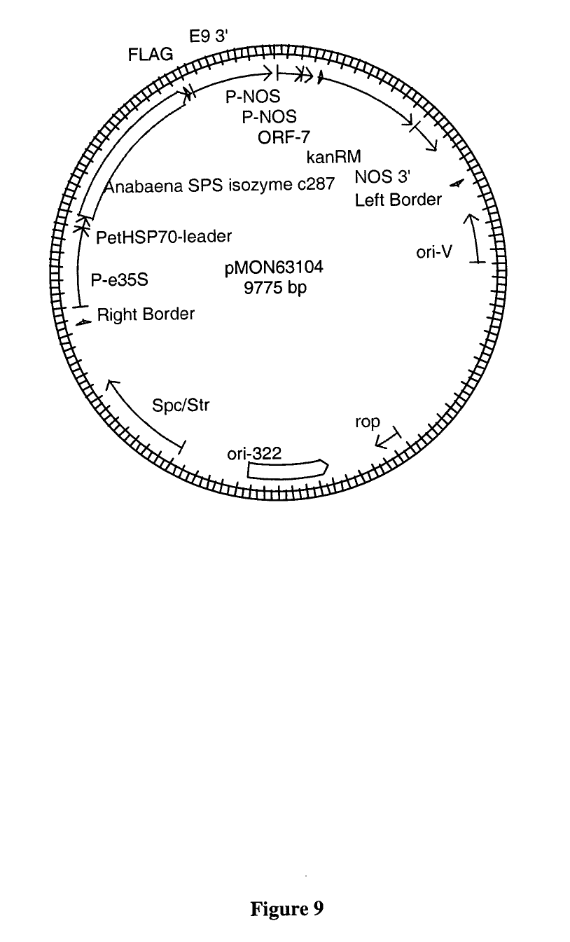

PCR products were obtained utilizing primers C 154F (SEQ ID NO: 15) and C154R3 (19) and C287F (17) and C287R2 (20) in reactions with vectors pMON 63101 (FIG. 6) and pMON₆₃O₂ (FIG. 7) as templates, respectively. The 5′ forward primers were identical to those used above, while the 3′ primers were designed to remove the translational stop codon and to allow insertion of the C-terminal HisTAG and Flag tags in the appropriate reading frame in their respective vectors. The results of the above PCR reactions were analyzed by analytical agarose gel electrophoresis (1.0% TAE). Products were then purified using Qiagen PCR clean up kits following the manufacturer's protocol. Recovered products were digested with NcoI and EcoRI and purified by gel electrophoresis (0.7-1.0% TAE). A partial digestion was performed on PCR product from C154 primer set since the gene contained an internal EcoRI site. The vectors pET-28b and pMON23450 pET-28b were digested with the same enzymes and treated with CIP prior to gel purification. Samples were again gel purified and the appropriate band was selected from analytical gel electrophoresis for subsequent cloning. Digested vector and PCR products from the excised bands were then recovered using the Gene Elute columns (Sigma, St. Louis) following the manufacture's protocol. The recovered purified vectors and fragments were then ligated using T4 Ligase (NEB) under standard conditions or by utilizing the rapid ligation kit (Roche, US) following manufacturer's protocol. The resultant ligation mixture was electroporated or chemically transformed using manufacturer's suggested protocol (Gibco, BRL) into DH5α or DH10β cells for propagation of the DNA. The transformed cells were plated on the appropriate antibiotic (pET-28, Kan and pMON23450) and resultant colonies were selected by colony PCR using the same PCR primers that were used to produce the insert. Plasmids were further confirmed by miniprep purification and specific restriction digestion to remove the inserted fragment. Whenever PCR was used to amplify insert they were further verified by cycle sequencing (GSC, standard protocols) using primers as described in Table 4.

### Example 6

### Overexpression in E. coli

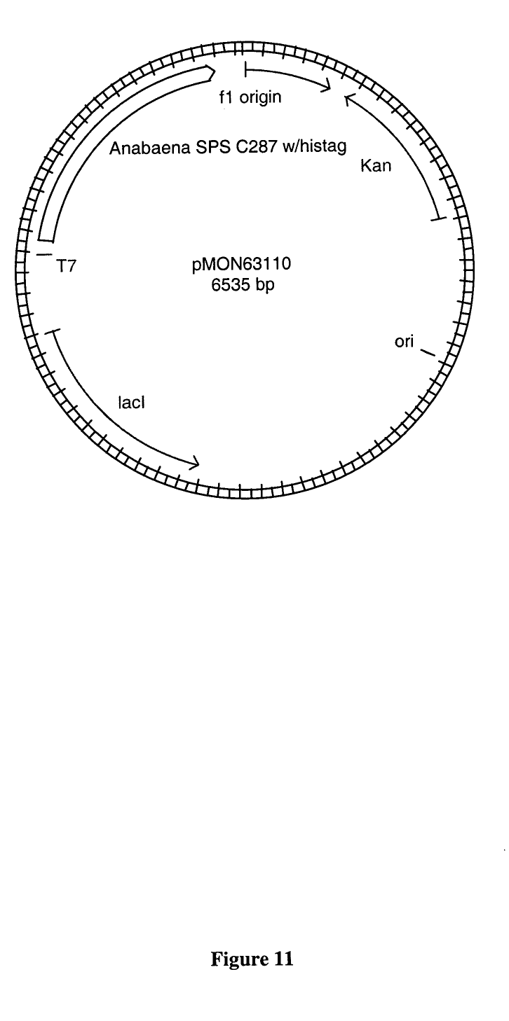

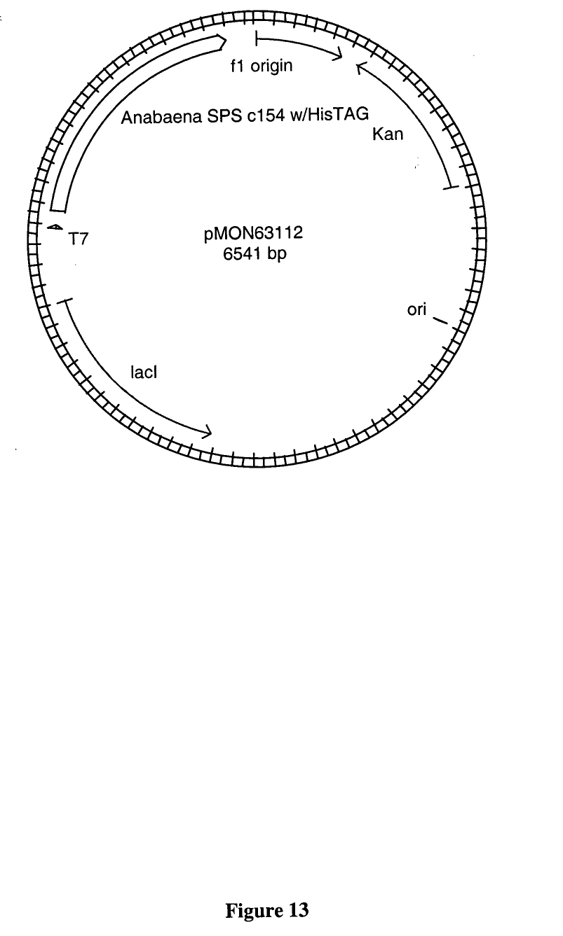

Over expression analysis for these constructs was carried out under essentially standard conditions as suggested by the manufacturer (Novagen, WI). Briefly, the pET-28b E. coli expression constructs, i.e., pMON63101, Anabaena SPS C154 pET-28b, pMON63102, Anabaena SPS C287 pET-28b, pMON63110 (FIG. 11), Anabaena SPS c287 with C-histag pET-28b, and pMON63112 (FIG. 13), Anabaena SPS c154 no stop with C-Histag pET-28b, were utilized in E. coli overexpression studies. These vectors were transformed into BL21DE3 cells that harbored the T7 RNA polymerase gene required for protein expression from these vectors (Novagen, WI). A 3 mL starter culture of these cells in LB/Kan was grown for 8 to 12 hours after which 2.5 mL of this culture was added to a fresh sample of LB/Kan (100 mL). These cells were grown at 37° C. with shaking at 200 rpm to an OD 600 nm 0.9-1.2 at which time they were induced with 0.5 mM IPTG. Cells were grown for an additional 3 to 4h, harvested by centrifugation (6500×g) and stored at −80° C. until analysis.

### Example 7

### Expression Analysis and Activity Determination

The resultant cell pellets were analyzed by SDS-PAGE (Laemmli, Nature 227: 680-685, 1970) for the presence of the bands expected for an overproduced protein with an apparent molecular weight of approximately 47 Kda. Cell extracts were made by sonication (Branson Model 150 50% duty cycle, power level 1, 3×30 second on ice) in 50 mM Tris-HCl pH 7.5 200 mMNaCl, 0.5% CHAPS and 2 mM AEBSF. Typically approximately 100 μL of extract buffer were used per 10 mL of cell culture (prior to centrifugation). These crude extracts were tested for SPS activity (assay as described in assay section below) and analyzed for protein concentration (Bradford, Anal. Biochem. 72: 248-254, 1976).

Radio HPLC activity assays containing 30 mM Bis Tis pH 6.5, 0.5 mM EDTA, 10 mM F6P and UDP-glc, 5 mM or 10 mM MgCl₂ and 5 μL of enzyme extracts (25 μL total volume) were run for 10, 15 or 30 min at 30° C. and quenched with 100 mM NaOAc 95% ethanol, pH 4.7, to a total volume of 200 μL. Quenched reactions were centrifuged at 14000×g for 5 minutes to clear solutions and pellet any debris in preparation for injection. One quarter of this mixture (50 μL) was analyzed by HPLC (HP 1100 System interfaced with a Packard Flo-One Model D515 flow scintillation detector) injected onto a Synchropak AX-100 anion exchange column (250×4.6 mm) running at a flow rate of 1.0 mL/min with 70 mM NaH₂PO₄/NaOH pH 4.8 mobile phase. This isocratic elution affords very clear separation of UDP-glc (ca. 12.5 min) from S6P (ca. 5.5 min). Controls contained substrates only and/or E. coli extract in the identical extraction buffer incubated and quenched under the same conditions. The activity assays (duplicate or triplicate) determined the percent turnover (quantitation of the ratio of the respective peaks) of UDP-glc to S6P for a given injection. Specific activity is reported in U/mg (U=μmol/min). Radiolabled Uridine-diphospho-D-[U¹⁴C] glucose, ammonium salt (UDPglc, 0.025 to 0.050 μCi per reaction) with a specific activity of 330 mCi/mmol was used in these assays.

LC-MS analysis was also used to confirm the presence of S6P product. A typical reaction assay samples as described above with and without enzyme added were submitted for LC-MS analysis. These samples were quenched with 100% EtOH only instead of the normal quench.

Activity Evaluation

Two putative Anabaena SPS genes were cloned and overexpressed and the activity (specific enzymatic function) confirmed. The constructs pMON63101 and pMON 63102 contained C154 (SEQ ID NO: 1) and C287 (SEQ ID NO: 3) inside pET-28b (+) expression vectors. Both C154 and C287 contain genes that encode active SPS enzymes as expressed in E. coli, determined by crude extract analysis (FIG. 2a). In an effort to further analyze these activities these two SPS genes from Anabaena were C-terminal His-Tagged (pMON63110, and pMON63112). Proteins from Anabaena c154 (pMON63111 and pMON63109) were purified on 10% and 12% SDS-PAGE gel, respectively, using IMAC and a step gradient in imidazole 50, 250, 500 mM. The SPS proteins came off in 250 mM range. Samples were then gel filtered into enzyme reaction buffer for further use and storage (−80° C.). The SPS purification made it possible to determine the specific activity of these genes in a purified state as well as evaluate the affects of a C-terminal fusion (i.e., flag for plant constructs) on the activity of these genes. The results demonstrate that these genes are active with this modification. The highest specific activities calculated for the purified SPS proteins are 16.4 U/mg for C154 and 6.5 U/mg for C287 (U=umol/min). These numbers are consistent in magnitude with the reports of purified Anabaena SPS (Porchia et al., Proc, Natl. Acad. Sci. USA 93: 13600-13604, 1996).

Furthermore the product of the reaction was unequivocally identified by LC-MS analysis to be S6P (FIG. 2b). Additional characterization of these enzymes revealed that they do not turnover UDP-glc to glucose (SS activity) when fructose is used in place of F6P in the standard assay demonstrating a key distinguishing feature of SPS enzymes, selectivity for F6P.

### Example 8

### Protein Purification

Both Anabaena SPS proteins were purified by utilization of a HisTag fusion to the C-terminal end of the protein. Samples were extracted as above for activity assays. Purification was carried out following the manufacturer's protocol for gravity purification with the exception that elution was performed in 250 mM Imidazole instead of 500 mM (Pharmacia, HisTrap Column, 1.0 mL). Gel filtration was carried out on a PD-10 columns following manufacture's directions (Pharmacia) to exchange buffer from the high imidazole concentration of the Histag purified samples into 30 mM Bis-Tris pH 6.5 0.5 mM EDTA, 0.1% CHAPS for activity assays and storage. Samples were subject to activity assays as described above as well as analysis by SDS-PAGE.

Phosphate Inhibition

Assay of the HisTag purified proteins were performed using standard assay conditions except in the initial volume (40 uL) and with the addition of phosphate (0 to 80 mM) to the reaction mixture. All reactions were run in triplicate.

Incorporation of the Histag has allowed nearly complete purification of these SPS proteins enabling the determination of sensitivity to phosphate inhibition (Stitt et al., In: The Biochemistry of Plants, Vol. 10: 327-409, 1987; Doehlert & Huber, Plant Physiol. 73: 989-984, 1983). Estimates from these results indicate that these gene products are approximately 50% less sensitive to phosphate inhibition when compared to other plant species, for example, wheat (FIG. 3).

Sequence Comparison

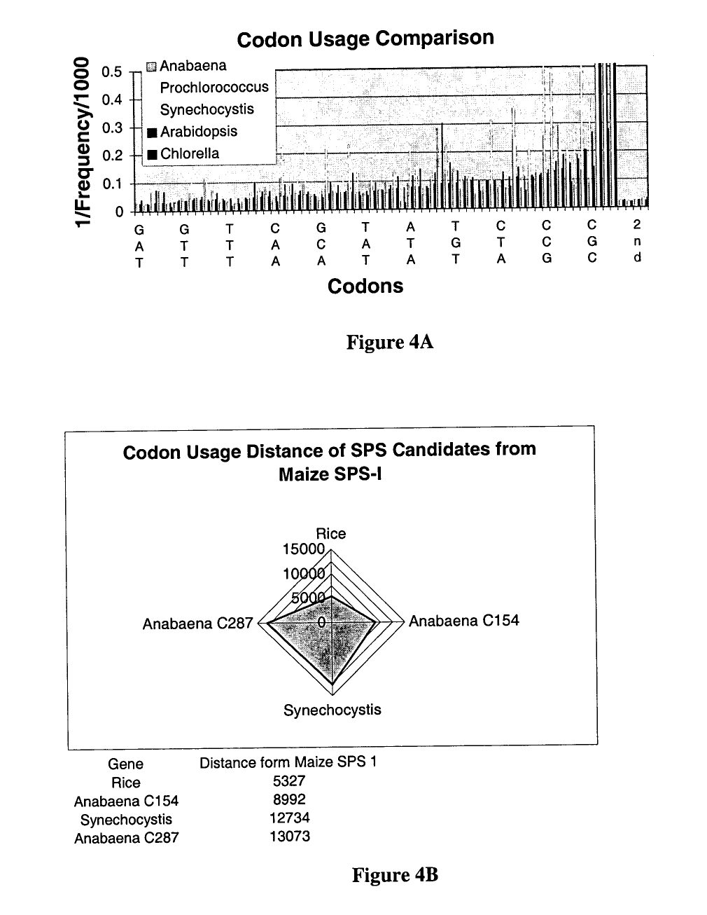

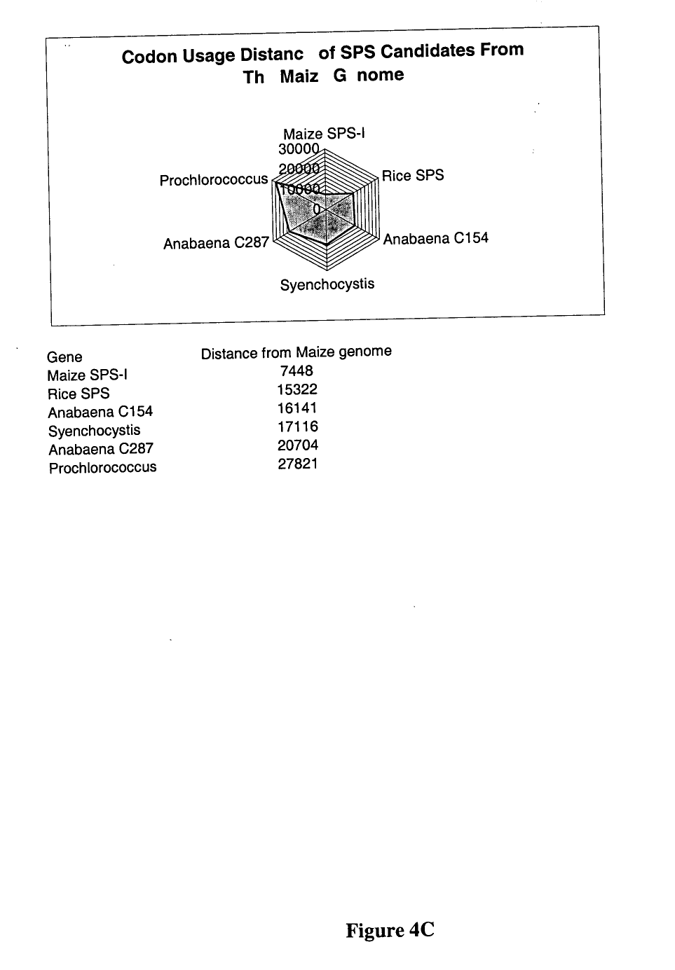

As mentioned above unique characteristics of these Anabaena sequences were highlighted by their comparison to plant and other cyanobacterial species (see FIG. 1 and Table 1). First, these proteins do not contain regulatory phosphorylation sites (see FIG. 1; Toroser et al., Plant J. 17: 407-13, 1999; McMichael et. al., Arch Biochem Biophys. 307: 248-52, 1993; Huber & Huber, Biochem J. 283: 877-82, 1992). Furthermore, these sequences differ with published assertions about invariant residues for SPS proteins as highlighted in alignment in FIG. 1 (Curatti et. al., Planta 211: 729-735, 2000). These genes encode proteins that are small in size relative to even other cyanobacterial SPS genes. Anabaena cDNA also has codon usage that is most amenable to expression in Arabidopsis (FIG. 4).

SPS Genes from Other Cyanobacterial Species

The identification and confirmation of these unique forms of Anabaena SPS has allowed further annotation, identification and isolation of other distantly related SPS genes from Prochlorococcus marinus, Nostoc punctiforme, and Synechococcus. It is clear from the arrangement of hits when compared to the results from Prochlorococcus and Anabaena that Nostoc has similarities to Anabaena and that Synechococcus is more like Prochlorococcus and Synechocystis. For example Anabaena and Nostoc have at least two SPS and SS genes while Synechocystis has one SPS and no obvious SS genes. For Nostoc, the search with sucrose synthase separates the top hits (one of them) from the secondary hits (SPSs) as in Anabaena.

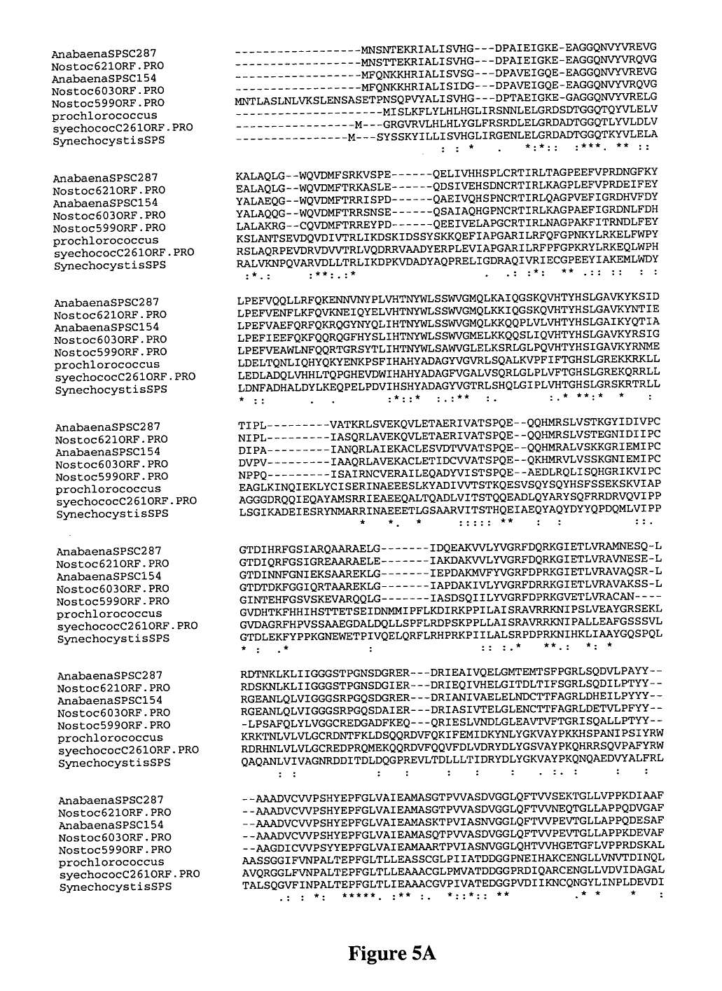

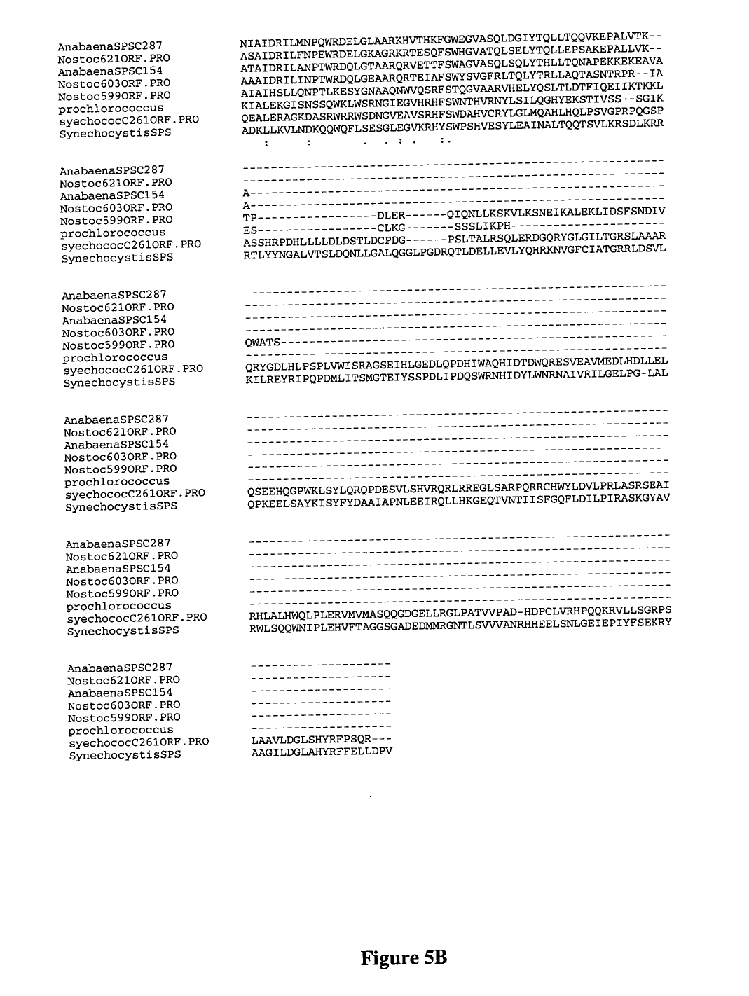

Based on these blast results the contigs were retrieved and the open reading frames located and compared to the other SPS proteins. The results of that comparison indicated that for Synechococcus contig 261 did indeed contain a SPS. As for Nostoc, contigs 599, 603, and 621 were all SPS genes as determined by sequence homology to other SPS genes from the same clade of a protein tree. An alignment of only cyanobacterial genes follows in FIG. 5. FastA sequences for the coding DNA and proteins have been entered in the sequence section.

The genomic DNAs containing SPS genes from Prochlorococcus marinus, Nostoc punctiforme, and Synechococcus will be sequenced and the subsequent activity assays conducted based upon the procedures for Anabaena SPS genes. The primers used for PCR and sequencing these cyanobacterial SPS genes are listed below in Table 6.

TABLE 6

Primers for isolation of Prochlorococcus marinus, Nostoc punctiforme,
and Synechococcus SPS genes.
Table 6A. PCR primers:

| | | | | SEQ ID |
|---|---|---|---|---|
| PCR primers all 5′ to 3′ | Primer name | Organism | Sites | Numbers |
| AGATCT CC ATG GCT AGT TTG | PrclFP check on | Prochlorococcus | Bgl ll, | SEQ ID NO: 33 |
| AAA TTT TTA TAT TTA CAT TTG | | | NcoI | |
| GCGAATTCTCGAGTCA ATG GGG | PrclR2 | Prochlorococcus | EcoRI, | SEQ ID NO: 34 |
| TTT TAT AAG TG | | | XhoI | |
| AGAGAGAATTCC TGC ATG GGG | PrclR3ns | Prochlorococcus | EcorI | SEQ ID NO: 35 |
| TTT TAT AAG TG | | | in | |
| | | | frame | |
| G/AAT/TC EcorI AAT in middle has to | | | | |
| be in frame | | | | |
| GTAAGATCTGCCACC ATG GGA | SyncspF | Synechococcus | Bgl ll, | SEQ ID NO: 36 |
| AGG GGT GTC CGT G | | | NcoI | |
| TAGA GAATTCAAGCT TCA GCG | SyncspR | Synechococcus | EcorI/ | SEQ ID NO: 37 |
| CTG ACT GGG AAA CCG | | | HindII | |
| | | | I | |
| AGA GAG AAT TCC GCG CTG ACT | SyncspRns | Synechococcus | EcoI | SEQ ID NO: 38 |
| GGG AAA CCG | | | in | |
| | | | frame | |
| GTA AGA TCT GCC ACC ATG GCT | Nos599F | Nostoc | BglII, | SEQ ID NO: 39 |
| ACT CTT GCT TCT TTA AAT | | | NcoI | |
| TAGA CTCGAGAAGCT TTA ACT | Nos599R | Nostoc | XhoI/ | SEQ ID NO: 40 |
| GGT TGC CCA CTG | | | HindII | |
| | | | I | |
| TAGA CTC GAG ACT GGT TGC | Nos599Rns | Nostoc | XhoI | SEQ ID NO: 41 |
| CCA CTG | | | in | |
| | | | frame | |
| XhoI CTC/GAG CTC GAG must be in | | | | |
| frame | | | | |
| GTAAGATCTGCCACC ATG GTC | Nos603F | Nostoc | BglII, | SEQ ID NO: 42 |
| CAG AAT AAG AAA C | | | NcoI | |
| TAGA CTCGAGAAGCT TTA AGC | Nos603R | Nostoc | XhoI/ | SEQ ID NO: 43 |
| TGC AAT CCG GGG | | | HindII | |
| | | | I | |
| TAGA CTC GAG AGC TGC AAT | Nos603Rns | Nostoc | XhoI | SEQ ID NO: 44 |
| CCG GGG | | | in | |
| | | | frame | |
| GTAAGATCTGCCACC ATG GCC | Nos621F | Nostoc | BglII, | SEQ ID NO: 45 |
| TCT ACC ACC GAA AAA CG | | | NcoI | |
| TAGA CTCGAGAAGCTT CTA TTT | Nos621R | Nostoc | XhoI/ | SEQ ID NO: 46 |
| AAC AAG CAA TGC AGG | | | HindII | |
| | | | I | |
| TAGA CTC GAG TTT AAC AAG | Nos621Rns | Nostoc | XhoI | SEQ ID NO: 47 |
| CAA TGC AGG | | | in | |
| | | | frame | |
| GTA AGA TAT CAT ATG ACA ACC | AgroF | Agro | BglII/ | SEQ ID NO: 48 |
| ACG AGC GAA AC | | | NdeI | |
| TAGA CTC GAG AAG CTT TCA ATC | AgroR | Agro | XhoI/ | SEQ ID NO: 49 |
| GCC GTC ATT CCA TG | | | Hind | |
| | | | III | |
| TAGA CTC GAG ATC GCC GTC ATT | AgroRns | Agro | XhoI | SEQ ID NO: 50 |
| CCA TG | | | in | |
| | | | frame | |

TABLE 6B
Sequencing primers:

| | | |
|---|---|---|
| GAAATTGATAATATGATGATTC | PclrFseqp | SEQ ID NO: 51 |
| GGG ATA GGC CAC TTT TCC | PclrRseqp | SEQ ID NO: 52 |

### Example 9

### Protoplast Transformation Vector Construction

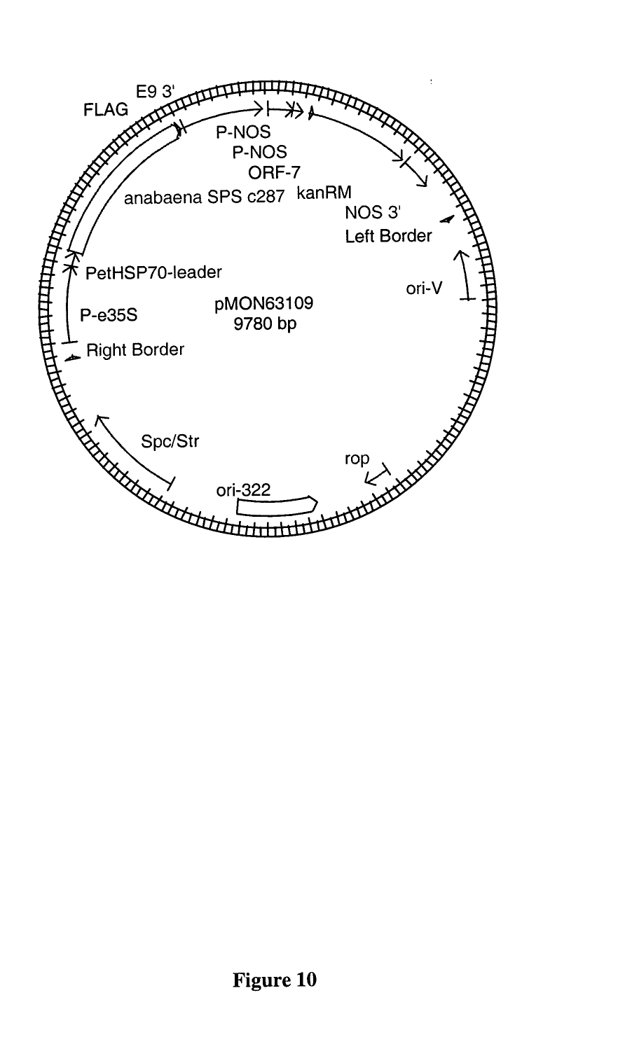

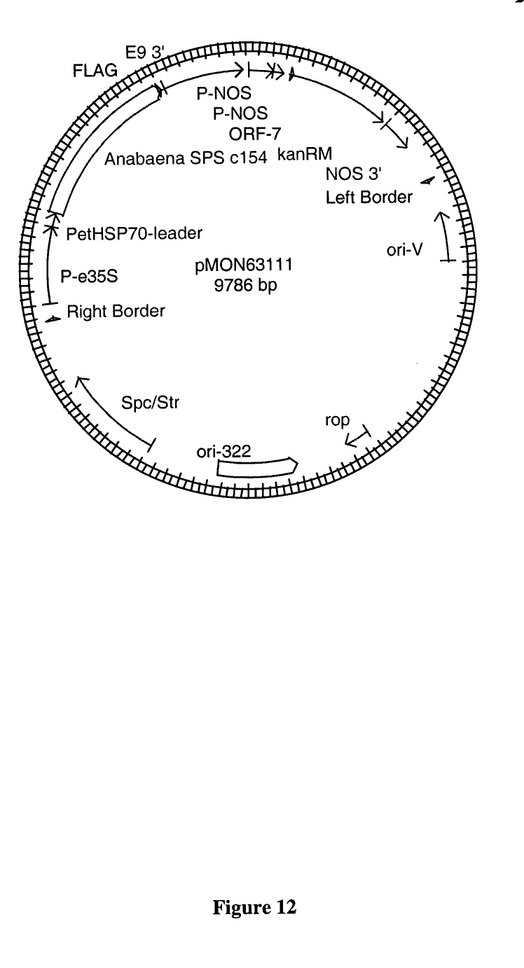

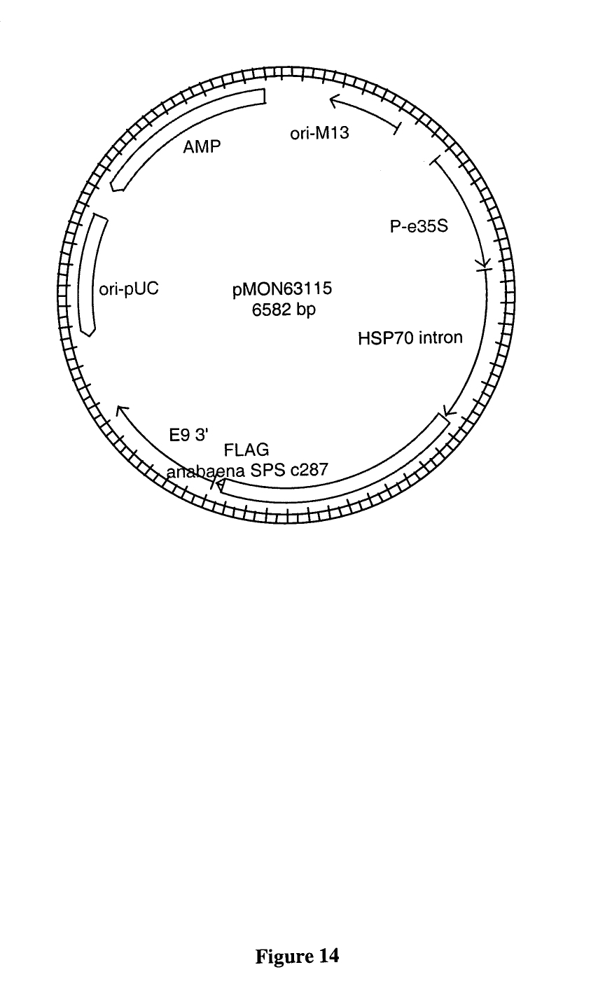

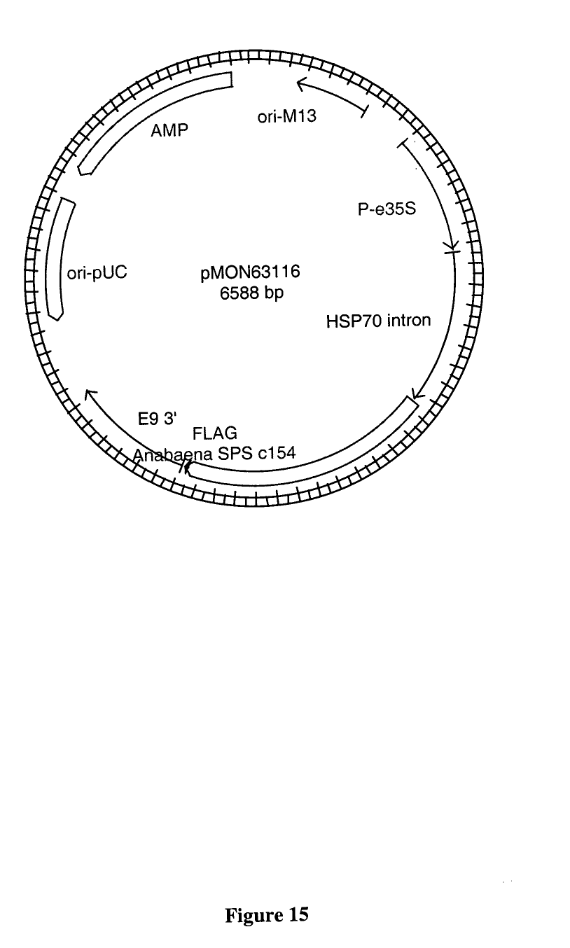

Protoplast expression vectors containing C287 and C154 Anabaena SPS genes were constructed by subcloning (standard conditions) the NcoI/SmaI fragment from digested pMON63109 (FIG. 10) and pMON63111 (FIG. 12), respectively, into pMON 13912 at the same positions to produce pMON63115 (FIG. 14) and pMON63116 (FIG. 15). Again vectors obtained from the ligation and subcloning step were isolated as above and confirmed by digestion.

### Example 10

### tBLASTN Search of Databases and Phrap Analysis of Results

A tBLASTN (Altschul et al., J. Mol. Biol. 215: 403-410, 1990; Altschul, et al., Nucleic Acids Res. 25: 3389-3402, 1997) search of PhytoSeq (Maize Seq) and BlastALL was performed utilizing the 5′ end of Spinach sucrose phosphate synthase (SPS1:gi12651081:1 gb|AAC60545.11, SPS [Spinacia oleracea]) to identify hits in the Maize database. The entire set of sequences was that subjected to Pharp (Incyte tools) clustering (default parameters) analysis to select 5′ clones that showed protein homology to the 5′ end of SPS and that were found in separate clusters. These clones were acquired and subjected to full insert sequencing.

Sequence Identification

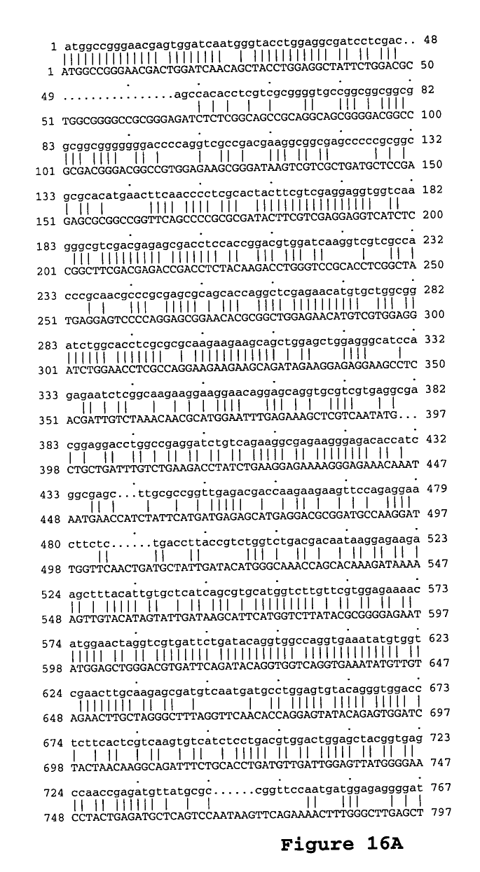

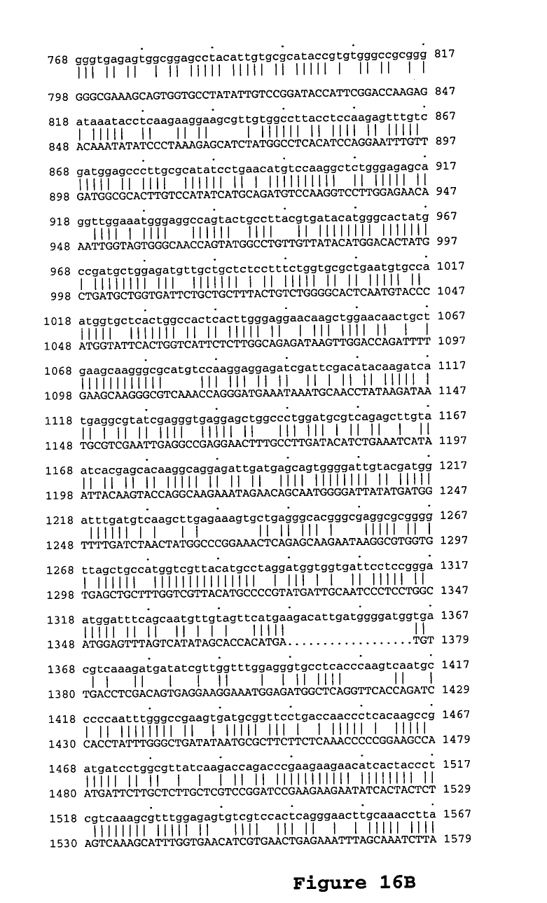

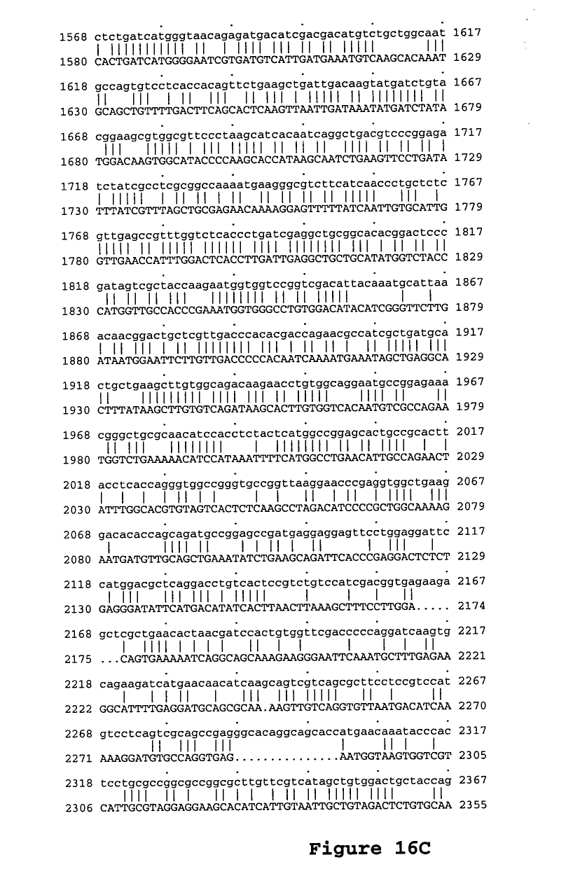

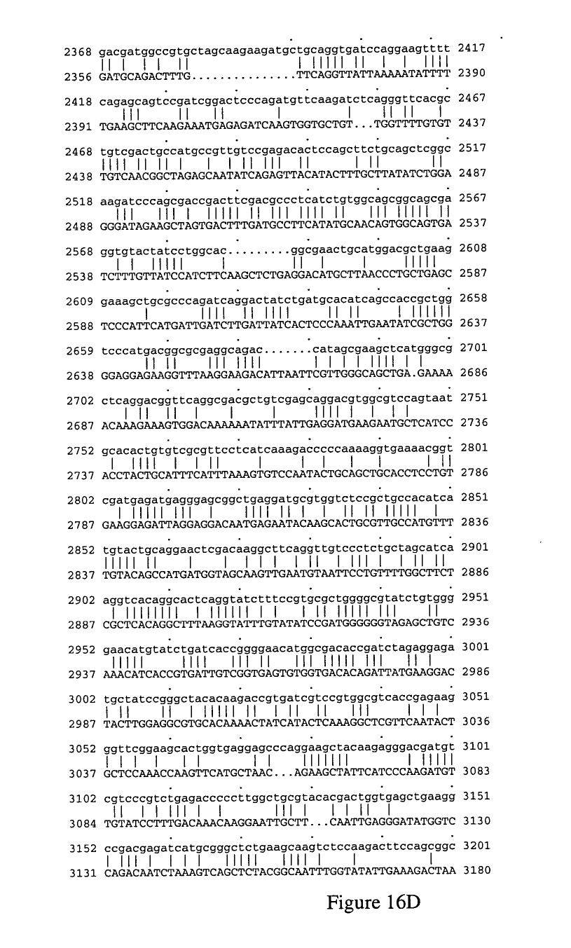

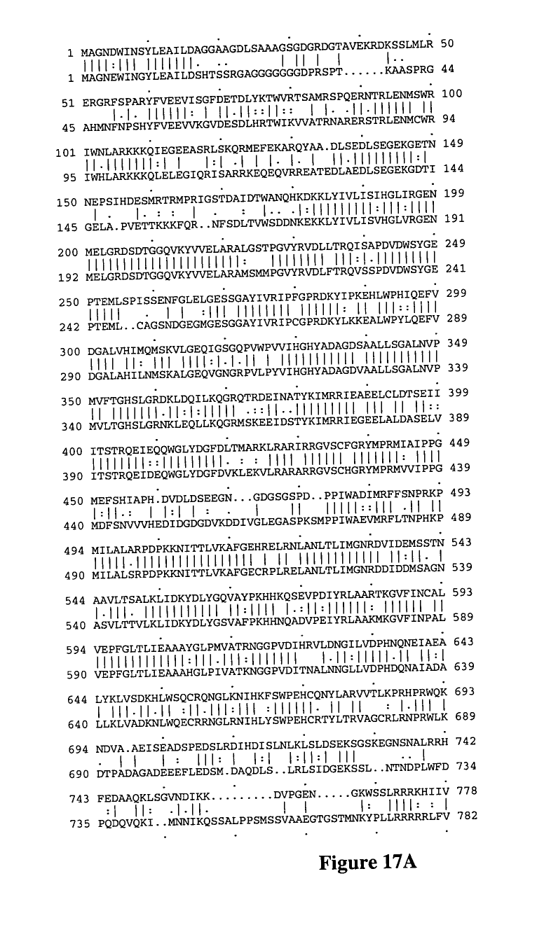

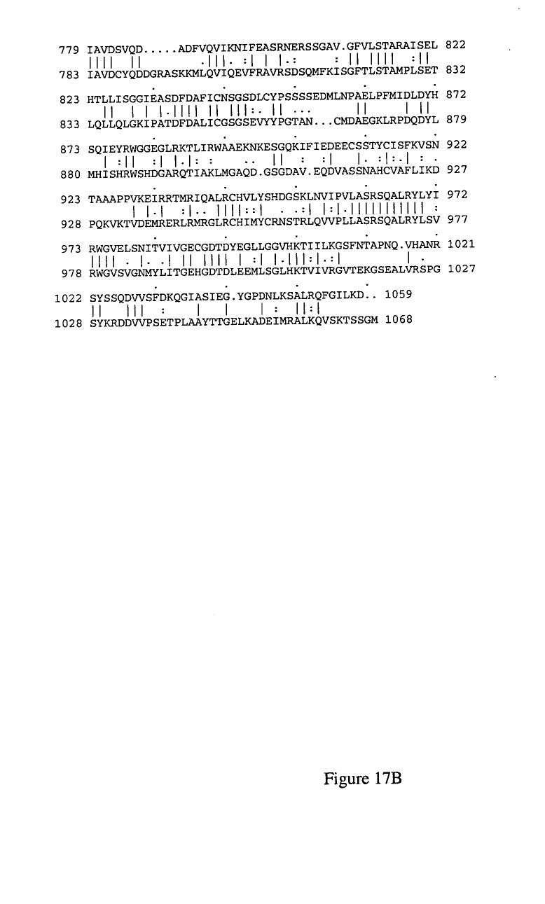

We have located a unique isozyme of maize SPS in our databases. This allele is significantly different at the DNA level not to group in the original Phrap clustering analysis. A GAP analysis of the DNA is shown in FIG. 16 and that of the protein in FIG. 17. This protein shares 55% identity with SPS 1 protein from maize. Considering that these sequences are from the same species this is a significantly different maize SPS gene.

### Example 11

### Sequence Analysis and Protein Alignments

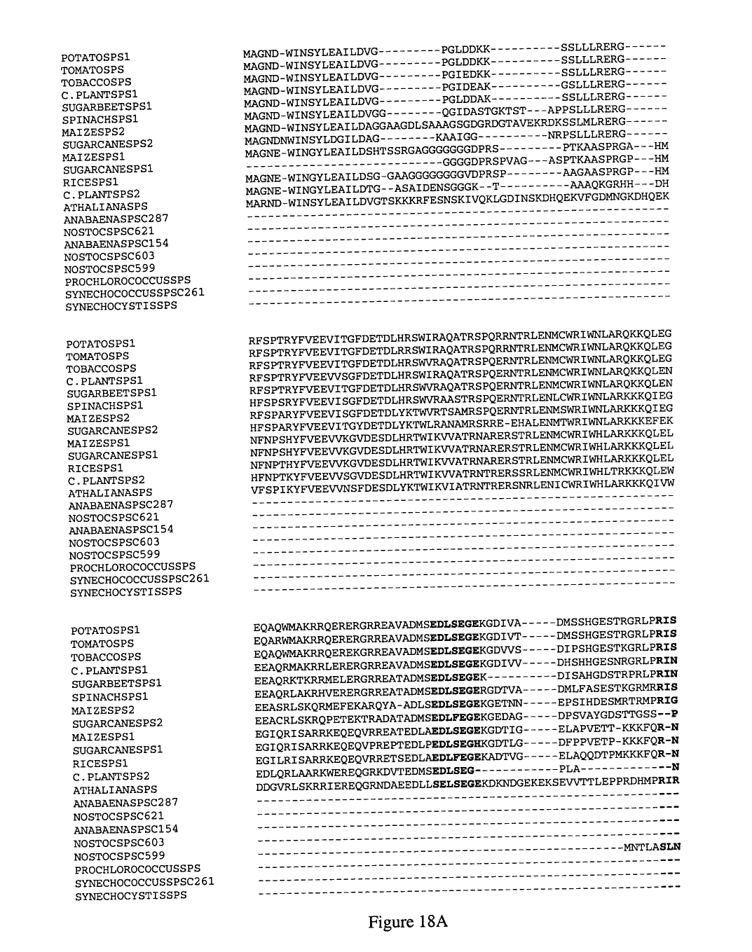

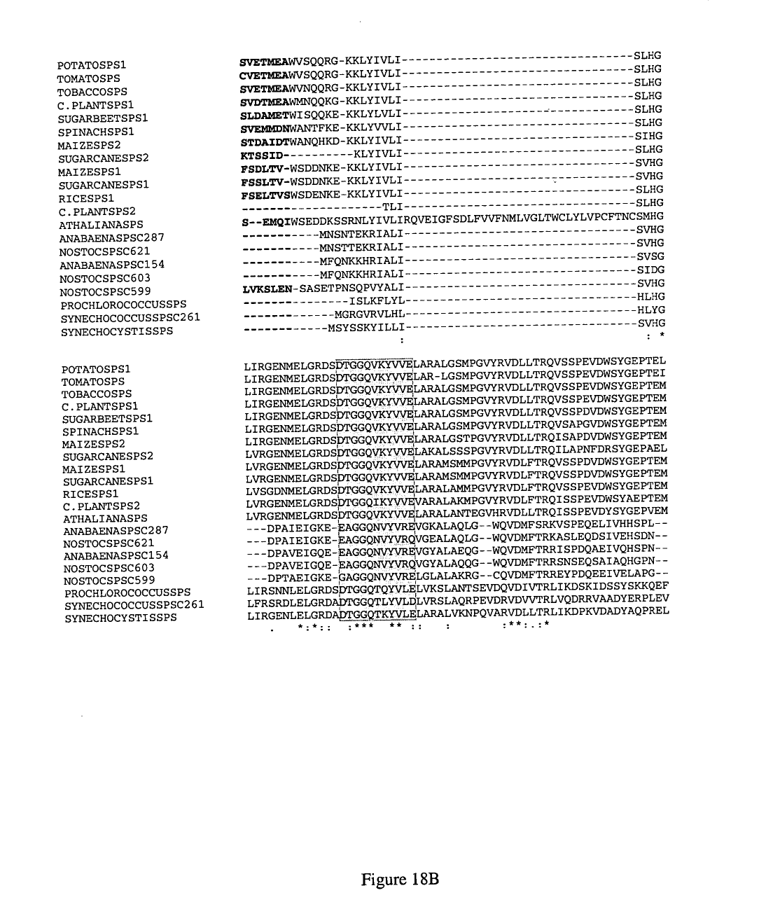

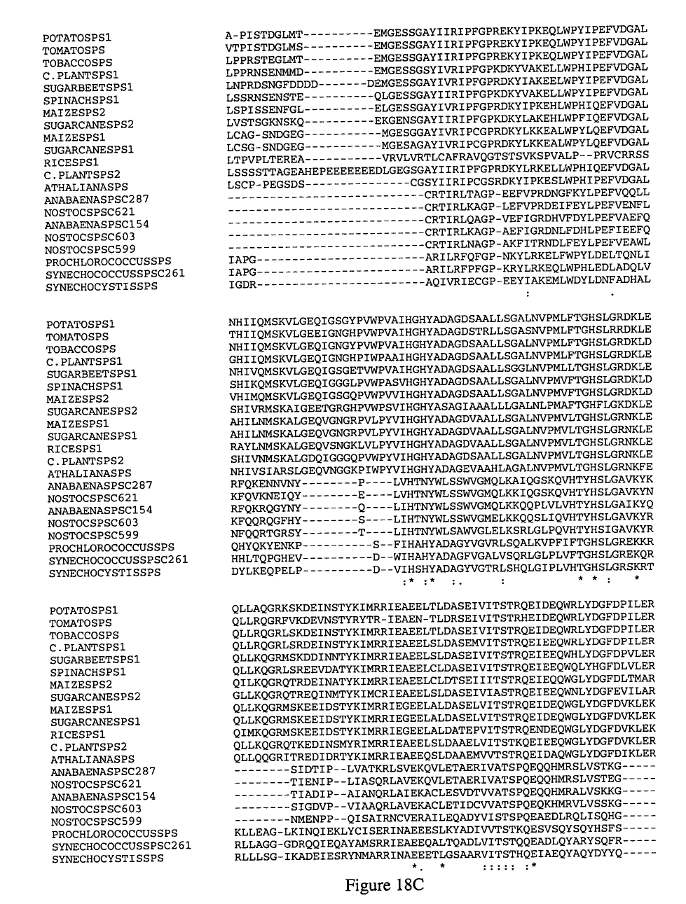

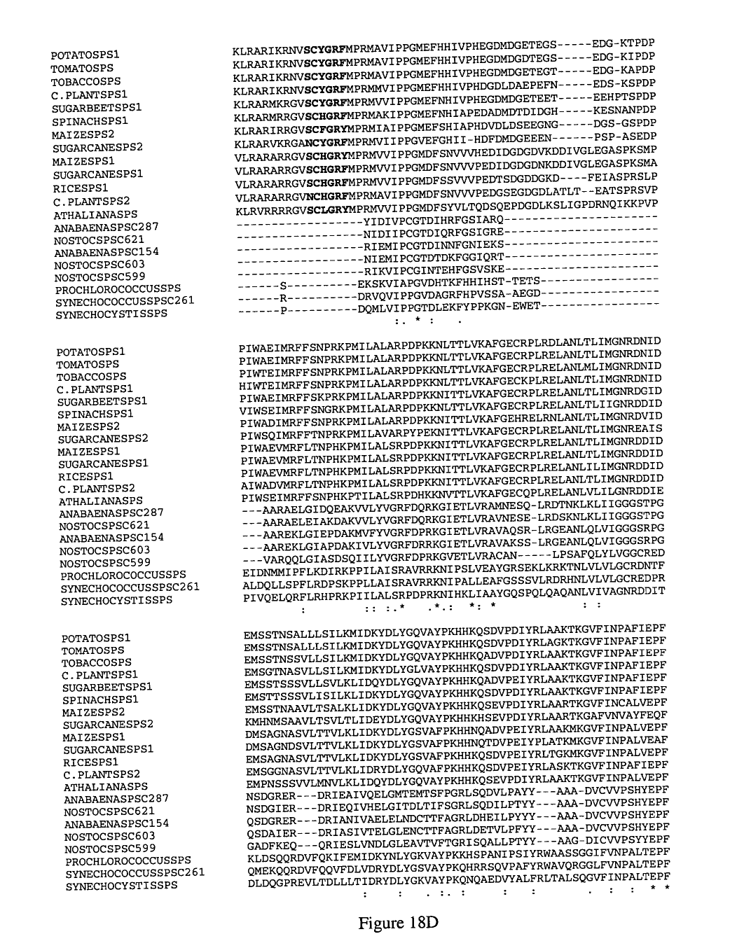

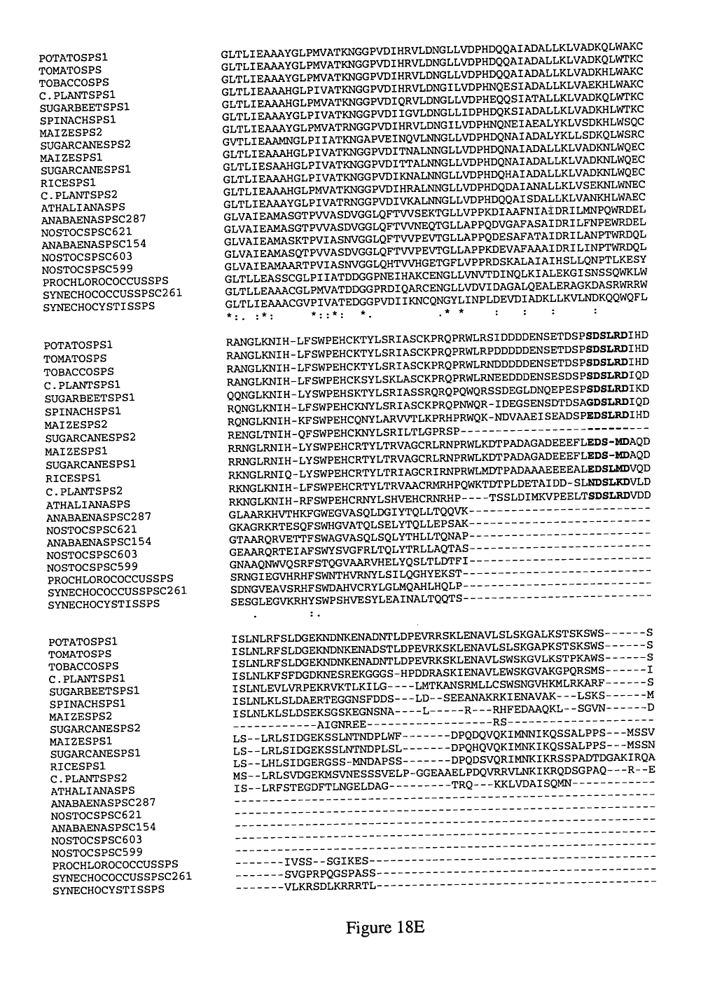

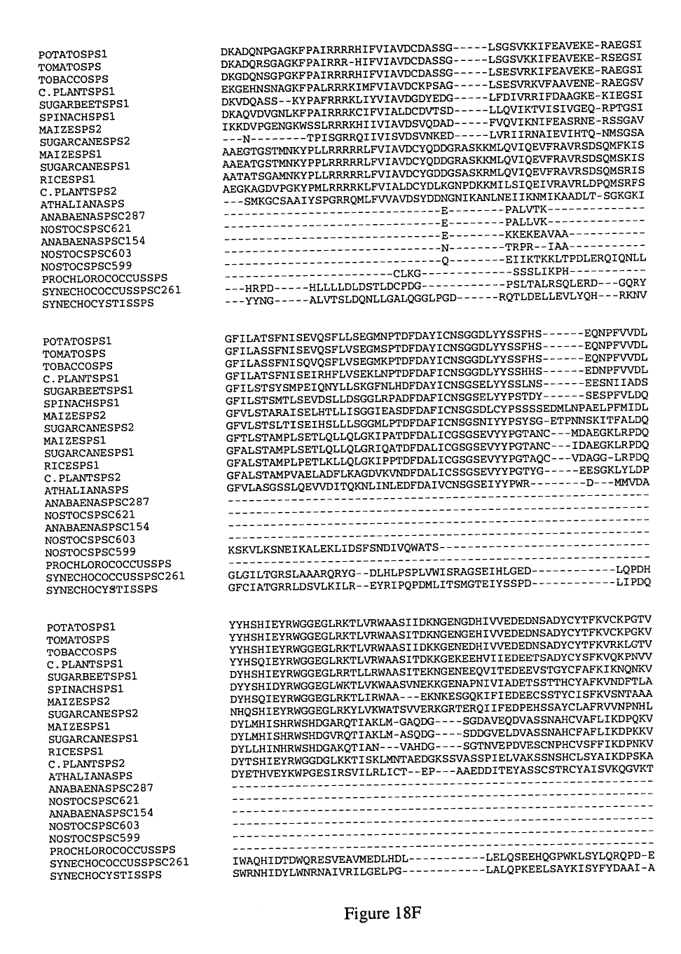

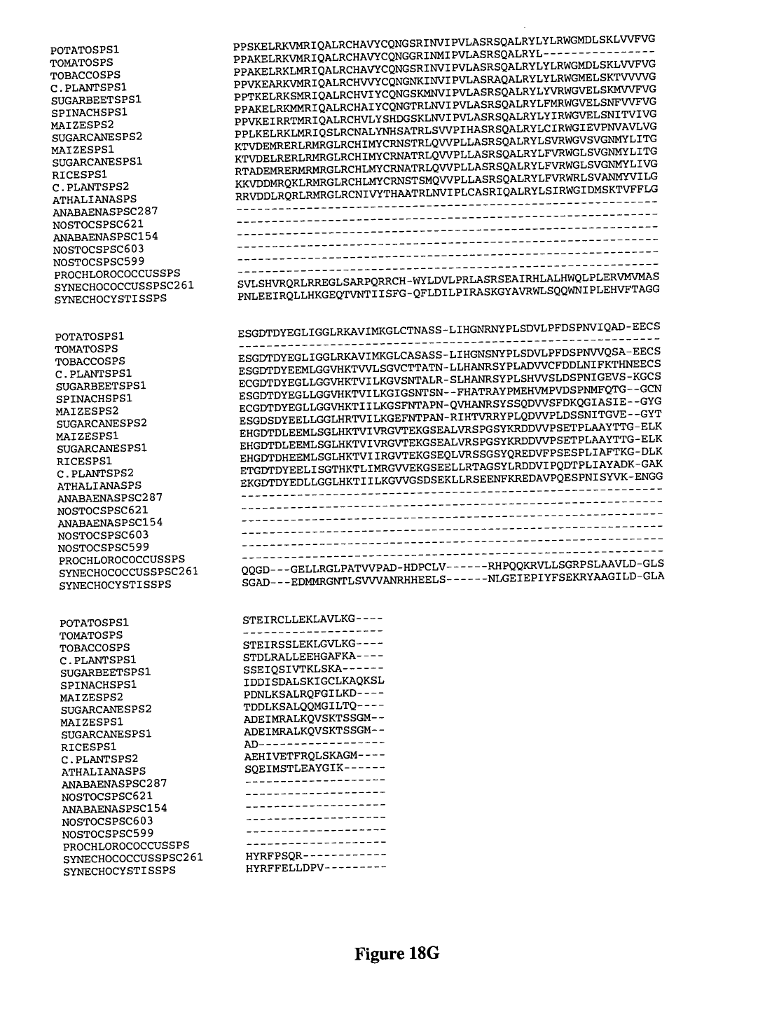

Protein alignment trees were created using Clustal X 1.8 (Thompson et. al., Nucleic Acids Research 24: 4876-4882, 1997). SPS amino acid sequences from a cyanobacterium (Synechocystis) and other higher plants including maize, rice, tomato, potato, sugarcane, sugarbeet, spinach and Arabidopsis thaliana were first aligned under default conditions for a complete alignment and adjustments made if necessary. These sequence alignments were then used to produce a Neighbor joining bootstrap protein tree in the same application using default parameters with exception of the use of 1000 iterations. The tree (not shown) showed that, although they were grouped together with other SPS proteins from higher plants, the maize SPS 1 and SPS2 were separated on different clades. This result suggests that they have some sequence differences. Gap (Needleman and Wunsch, GCG, Wisconsin Package, 1970) was used (default parameters) to compare the DNA and protein sequences in pairs. Provided in FIG. 18 is a multiple sequence comparison of maize SPS2 with maize SPS 1 and those SPS proteins from other higher plants.

### Example 12

### Sequencing and PCR Cloning of Maize SPS 2 Gene

All molecular biology was performed using standard protocols unless otherwise noted (Ausubel et al., Current Protocols in Molecular Biology, John Wiley & Sons, New York, N.Y., 2000); Sambrook et al., Molecular Cloning, A Laboratory Manual, Cold Spring Harbor Press, Plainview, N.Y., 1989). Library clones were received in pSport1 (Gibco, BRL) and these vectors were utilized as template in standard sequencing reactions. Primers used to perform PCR and to sequence 700072387H1 can be found in Table 4. PCR conditions for these manipulations included GibcoBRL Platinum Taq High Fidelity polymerase with its supplied buffer at suggested concentration, 2.0 mM MgSO₄, 10 uM primers, 10 ng template either 70002387pSport1 or pMON52915 in a total of 50 uL. PCR thermal cycling conditions were: initial denaturation at 94° C. for 2 minutes, followed by 94° C. for 30 seconds, with annealing at 55° C. for 30 seconds and extension at 68° C. for 3.5 repeating steps 2-4 35 cycles. Samples were then held at 4-10° C. until use.

The entire coding region of maize SPS found with EST 700072387H1 was sequenced out of pSport1 to 4 times coverage using the sequencing primers in Table 4. This sequence is the complete unmodified full length maize SPS (SEQ ID NO: 53). This sequence was used as a platform for the following manipulation.

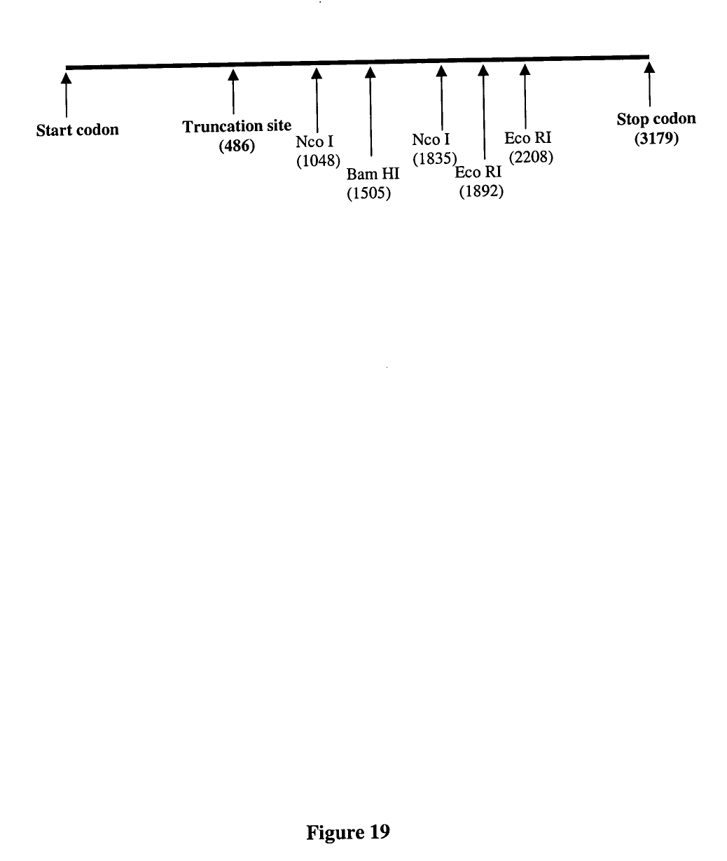

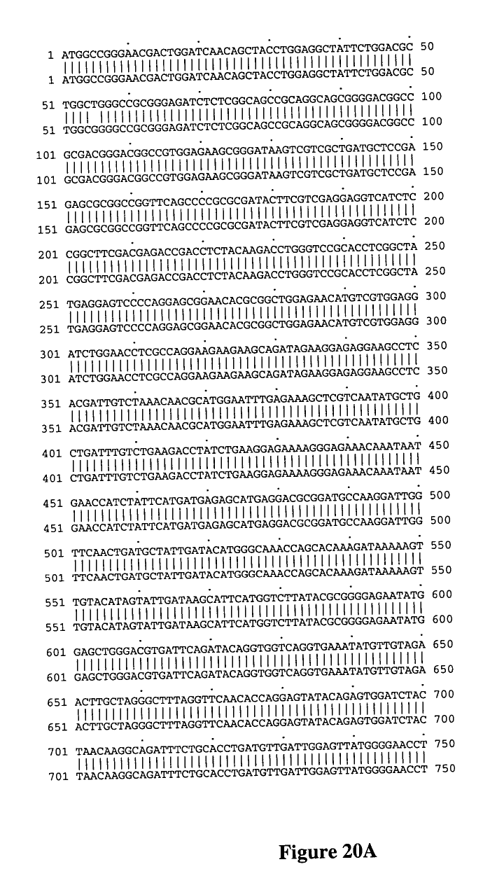

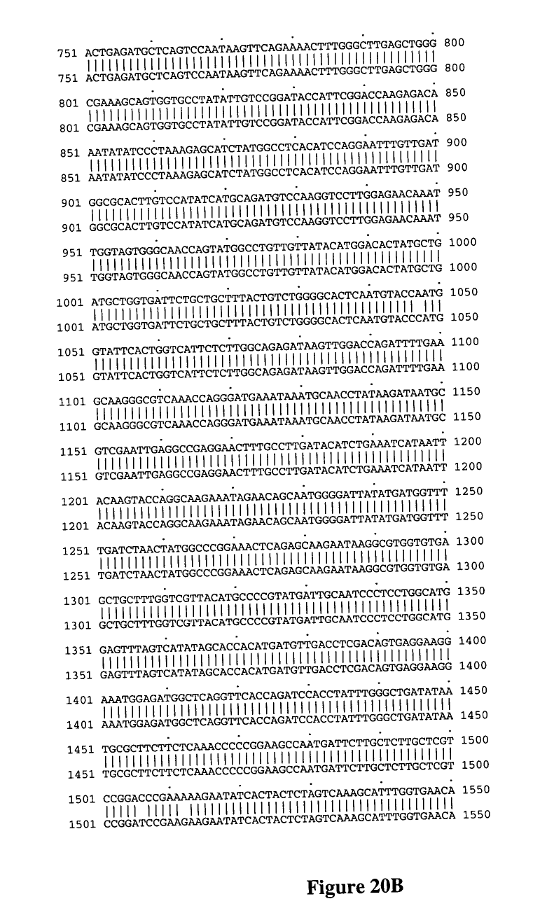

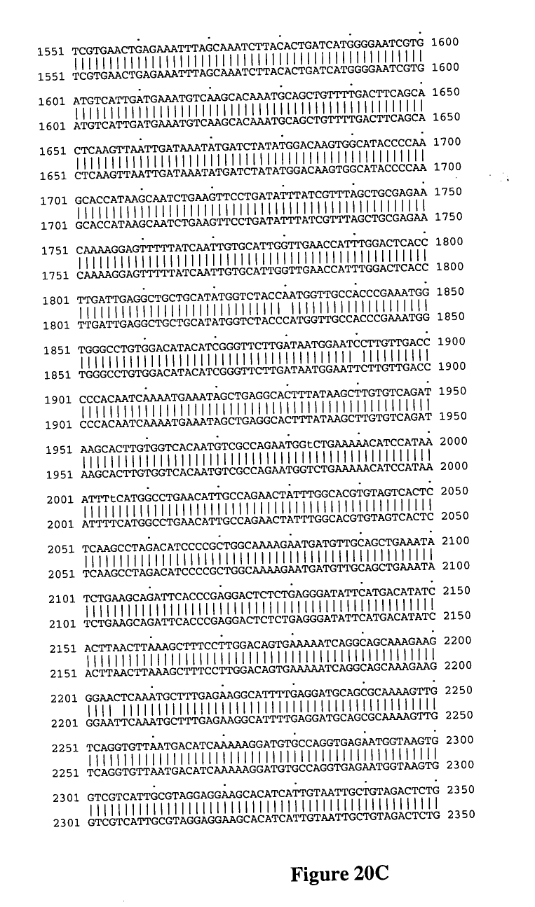

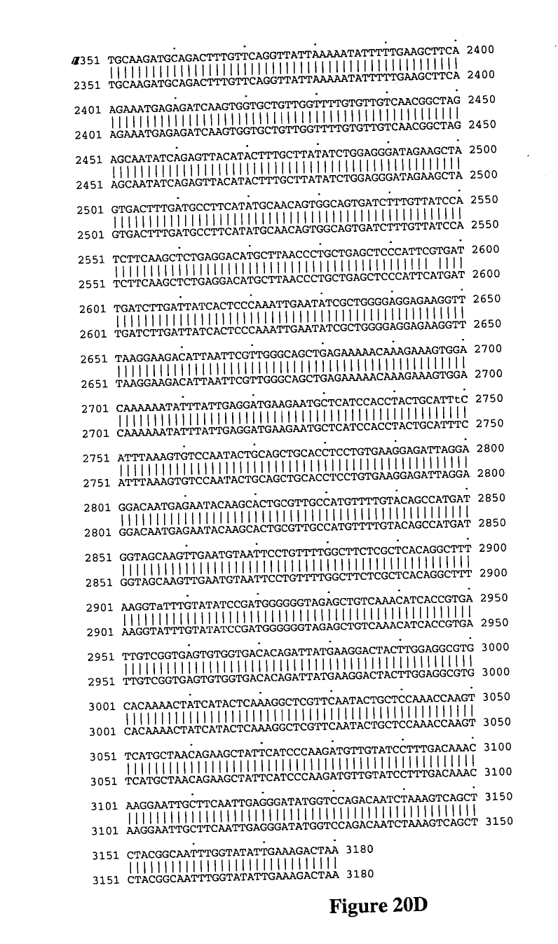

The maize SPS2 gene was modified to remove internal restriction enzyme sites in order to make it more amenable to subcloning. Mutagenesis was carried out with Stratagene's QuickChange site-directed mutagenesis kit (Catalog #200518) following the manufacture's standard protocol unless otherwise noted. All primers used can be found in Table 4. The first stage of modification was to remove five internal restriction enzyme sites (Nco I, BamH I, and EcoR I) detailed in the FIG. 19. BamH I 1505 was removed by making the point mutation from GGATCC to GGACCC, which did not change the protein coding sequence. The desired change was confirmed by a BamH I digestion. The PCR product was then used in a second round of modification to remove the Nco 1 (1048). This point mutation changed the CCATGG to CAATGG, which did not change the protein coding sequence. The mutation was confirmed by a Nco I digestion. The PCR product was then used in a third mutagenesis step to remove Nco I (1835). The point mutation changed the CCATGG to CAATGG, which did not change the protein coding sequence. The mutation was confirmed by a NcoI digestion. The PCR product was then used in a fourth mutagenesis step to remove Eco RI (1892). The point mutation changed the GAATTC to GAATCC, which did not change the protein coding sequence. The mutation was confirmed by a EcoRI digestion. This PCR product was then used in a fifth round of mutagenesis to remove Eco RI (2208). The point mutation changed the GAATTC to GAACTC, which did not change the protein coding sequence. The PCR product was then confirmed by a Eco RI digestion. This final product was the SPS2 gene with the internal restriction enzyme sites removed. Sequencing of this product proved that a deletion was introduced during the process by PCR error. This was repaired by using primers to insert the missing nucleotide into the sequence with Stratagene's QuikChange site-directed mutagenesis kit (Catalog #200518) and following their standard protocol. The PCR product was sequenced and the repair of the error was confirmed. The sequencing showed that the gene was at this point error free at the amino acid translation level with two exceptions. Residue 19 (codon) was changed from a glycine to a tryptophan and residue 866 (codon) was changed from a methionine to a valine (SEQ ID NO: 55). This gene is in the plasmid pMON52915. The comparison of the original and mutated SPS nucleotide sequences is shown in FIG. 20.

At this point, the gene was truncated since it has been observed with maize SPS 1 that truncation of the plant secretory leader sequence has provided better expression results in E coli. The first 486 bases of the coding region were removed and the remaining sequence was subcloned into Invitrogen's pCR2.1-TOPO vector. Restriction enzyme sites were added to the 5′ and 3′ ends for cloning (Nco I and Eco RI/Bam HI, respectively). These mutations facilitated cloning into both the overexpression vectors for E. coli overexpression and would allow for insertion into binary vector for plant transformation as well. This PCR product was then subcloned into Invitrogen's TOPO TA cloning kit using standard protocol supplied with the kit. This construct (pCR2.1-Topo tSPS-2) was fully sequenced. The product was made using the Sense Truncation and Antisense Truncation primers as listed in Table 4. Primers had NcoI sites incorporated into their 5′ regions and EcoRI incorporated into their 3′ region. Products from these reactions were analyzed by analytical agarose gel electrophoresis (1.0% TAE). Products were then purified using Qiagen PCR clean up kits following the manufacturer's protocol. Recovered products and pAWSM-YCAAD 1 (Monsanto vector) were digested with NcoI and EcoR I and purified by gel electrophoresis (0.7-1.0% TAE). pAWSM-YCAAD1 was treated with calf intestinal phosphatase (CIP) prior to gel purification. Gel purified digested vector and PCR products from the excised bands were then recovered using the Gene Elute columns from Sigma (St. Louis, Mo.) following the manufacturers protocol. The recovered purified vectors and fragments were then ligated using T4 Ligase (NEB) under standard conditions or by utilizing the rapid ligation kit (Roche) following manufacturer's protocol. The resultant ligation mixture was electroporated or chemically transformed using the manufacture's protocol (Gibco, BRL) into DH5α or DH0β cells for propagation of the DNA. The transformed cells were plated on the appropriate antibiotic and resultant colonies containing the insert of interest were selected by colony PCR using the same PCR primers that were used to produce original product. Plasmids were further confirmed by miniprep and specific restriction digestion to remove the inserted fragment. This deletion is called the A469 mutation (SEQ ID NO: 57).

TABLE 7

Primers for PCR and Sequencing.
All primers are from 5′ to 3′.
Table 7a. PCR primers.

| | | |
|---|---|---|
| Sense BamHI 1505 | GCTCTTGCTCGTCCGGACCCG | SEQ ID NO: 72 |
| | AAGAAG | |
| Antisense BamHI | GTGATATTCTTCTTCGGGTCC | SEQ ID NO: 73 |
| 1505 | GGACGA | |
| Sense NcoI 1048 | GGGGCACTCAATGTACCAATG | SEQ ID NO: 74 |
| | GTATTCACTGG | |
| Antisense NcoI | CCAGTGAATACCATTGGTACA | SEQ ID NO: 75 |
| 1048 | TTGAGTGCCCC | |
| Sense NcoI 1835 | GCTGCATATGGTCTACCAATG | SEQ ID NO: 76 |
| | GTTGCCACCCG | |
| Antisense NcoI | CGGGTGGCAACCATTGGTAGA | SEQ ID NO: 77 |
| 1835 | CCATATGCAGC | |
| Sense Ecor I | CGGGTTCTTGATAATGGAATC | SEQ ID NO: 78 |
| 1892 | CTTGTTGACCCCCAC | |
| Antisense Ecor | GTGGGGGTCAACAAGGATTCC | SEQ ID NO: 79 |
| 1892 | ATTATCAAGAACCCG | |
| Sense Ecor I | GGCAGCAAAGAAGGGAACTCA | SEQ ID NO: 80 |
| 2208 | AATGCTTTGAGAAGGC | |
| Antisense Ecor | GCCTTCTCAAAGCATTTGAGT | SEQ ID NO: 81 |
| 2208 | TCCCTTCTTTGCTGCC | |
| Sense Repair | GCTCGTCCGGACCCGAAGAAG | SEQ ID NO: 82 |
| | AATATCACTACTC | |
| Antisense | GAGTAGTGATATTCTTCTTCG | SEQ ID NO: 83 |
| repair | GGTCCGGACGAGC | |
| Sense | GGACGCCCATGGCAAG | SEQ ID NO: 84 |
| Truncation | GATTGG | |
| Antisense | GGATCCGAATTCTTAGTCTTT | SEQ ID NO: 85 |
| Truncation | CAATATAC | |

TABLE 7b
Sequencing primers

| Msps2p114 | CGTGGAGAAGCGGGATAAGTC | SEQ ID NO: 86 |
|---|---|---|
| Msps2p191 | TCGAAGCCGGAGATGACCT | SEQ ID NO: 87 |
| Msps2p422 | CTGAAGGAGAAAAGGGAGAAACA | SEQ ID NO: 88 |
| Msps2p992 | ACTATGCTGATGCTGGTGATTCTG | SEQ ID NO: 89 |
| Msps2p1535 | AAGCATTTGGTGAACATCGTG | SEQ ID NO: 90 |
| Msps2p2105 | AAGCAGATTCACCCGAGGACT | SEQ ID NO: 91 |
| Msps2p2565 | GGACATGCTTAACCCTGCTGAG | SEQ ID NO: 92 |
| Msps2p2875 | GTTTTGGCTTCTCGCTCACAG | SEQ ID NO: 93 |
| XAT553 | GTTTGTCAAAGGATACAACA | SEQ ID NO: 94 |
| | TCTTGG | |
| ZPU.conp449 | CCCCAGGAGCGGAACAC | SEQ ID NO: 95 |
| RS7607-2p446 | CCTGATGTTGATTGGAGTTATGG | SEQ ID NO: 96 |
| RS7607-1p421 | AGGTGCAGCTGCAGTATTGGACAC | SEQ ID NO: 97 |
| RS7673-3p468 | CATTCTCACCTGGCACATCCTT | SEQ ID NO: 98 |
| RS7748-1p414 | TCAAGAACCCGATGTATGTCCAC | SEQ ID NO: 99 |
| RS7748-3p413 | CGAATTGAGGCCGAGGAACT | SEQ ID NO: |
| | | 100 |

TABLE 8
Constructs made.

| Constructs | Description |
|---|---|
| PsportI 70002387 | Full length SPS2 cDNA |
| PM0N52915 | (topo vector with full SPS2, modified) |
| pCR2.1-Topo-tSPS2 | Truncated modified tSPS2 |
| pAWSM-tSPS2 | Truncated modified tSPS2 |

### Example 13

### Overexpression in E. coli.

Over expression analysis for these constructs was carried out under non-standard conditions. A much lower amount of IPTG (50 μM) was required to induce detectable expression. Briefly, a truncated maize SPS gene (SEQ ID NO: 57) in pAWSM E. coli. was transformed into MM294 cells. A 3 mL starter culture of these cells in LB/spec was grown for 8 to 12 hours after which 2.5 mL of this culture was added to a fresh sample of LB/spec (100 mL). These cells were grown at 37° C. with shaking at 200 rpm to an OD 600 nm 0.9-1.2 at which time they were induced with 50 μM IPTG. Cells were grown for an additional 2.5h, harvested by centrifugation (6500×g) and stored at −80° C. until analysis.

### Example 14

### Expression Analysis and Activity Determination

The resultant cell pellets were analyzed by SDS-PAGE (Laemmli, Nature 227: 680-685, 1970) for the presence of the bands expected for an overproduced protein with an apparent molecular weight of approximately 99.8 KDa (tSPS2). These proteins were not overproduced to the extent that observation by SDS-page was definitive. Cell extracts were made by sonication (Branson Model 150 50% duty cycle, power level 1, 3×30 second on ice) in 50 mM Tris-HCl pH 7.5 200 mMNaCl, 0.5% CHAPS and 2 mM AEBSF. Typically approximately 100 μL of extract buffer was used per 10 mL of cell culture (prior to centrifugation). These crude extracts were tested for SPS activity (assay as described in assay section below) and analyzed for protein concentration (Bradford, Anal. Biochem., 72: 248-254, 1976).

Radio HPLC Activity Assays containing 30 mM Bis Tis pH 6.5, 0.5 mM EDTA, 10 mM F6P and UDP-glc, 5 mM or 10 mM MgCl₂ and 5 μL of enzyme extracts (25 μL total volume), were run for 0.5 or 1 h at 30° C. and quenched with 100 mM NaOAc 95% ethanol, pH 4.7, to a total volume of 200 μL. Quenched reactions were centrifuge at 14000×g for 5 minutes to clear solutions and pellet any debris in preparation for injection. One quarter of this mixture (50 μL) was analyzed by HPLC (HP 1100 System interfaced with a Packard Flo-One Model D515 flow scintillation detector) injected onto a Synchropak AX-100 anion exchange column (250×4.6 mm) running at a flow rate of 1.0 mL/min with 70 mM NaH₂PO₄/NaOH pH 4.8 mobile phase. This isocratic elution affords very clear separation of UDP-glc (ca. 12.5 min) from S6P (ca. 5.5 min). Controls contained substrates only and/or E. coli extract in the identical extraction buffer incubated and quenched under the same conditions. Activity assays (duplicate or triplicate) determined the percent turnover (quantitation of the ratio of the respective peaks) of UDP-glc to S6P for a given injection. Specific activity is reported in U/mg (U=μmol/min). Radiolabled Uridine-diphospho-D-[U ¹⁴C]glucose, ammonium salt (UDPglc, 0.025 to 0.050 μCi per reaction) with a specific activity of 330 mCi/mmol was used in these assays.

Activity Analysis

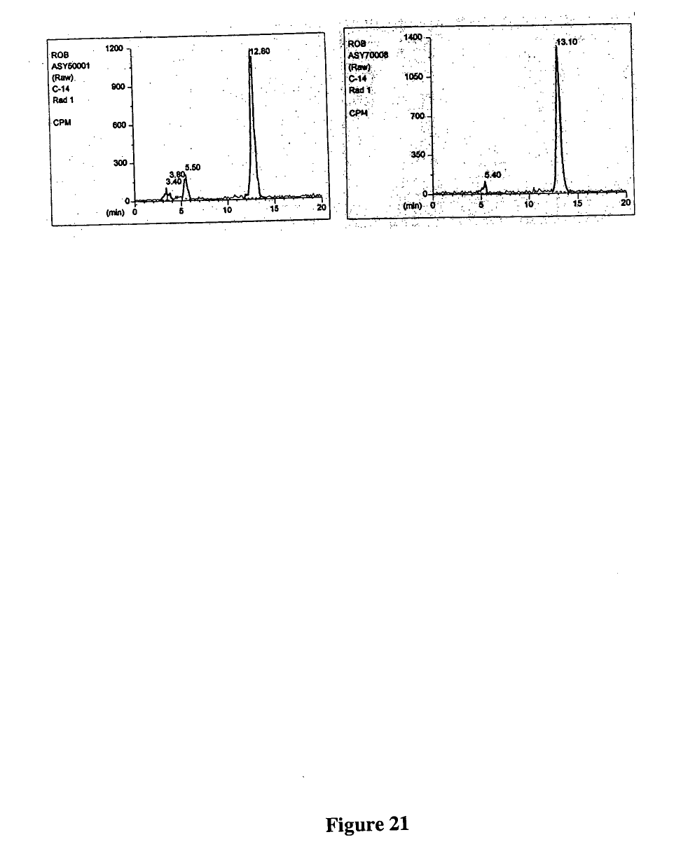

An activity analysis was performed to determine if this gene encoded an active viable SPS protein. We produced and used the modified tSPS2 gene because it facilitated movement of the gene and E. coli expression studies. These genes showed activity above background in the standard SPS enzyme assay (see Table 9 and FIG. 21). This analysis indicated that this gene was the viable SPS gene.

TABLE 9
Activity analysis

| | | | | | | Specific |
|---|---|---|---|---|---|---|
| | Vector | | | | | activity |
| | and | Cell | | | | (umol/min/ |
| Gene | Promoter | Line | Temperature | IPTG/OD | Duration | mg) |
| tSPS2 with | pAWSM | MM294 | 30° C. | 1.0 OD | 2.5 h | 0.01 |
| stop | ptac | | | 600 | | |
| | | | | 50 uM | | |
| | | | | 200 um | | |

### Example 15

### Protein Purification

MaizeSPS2 protein was purified by utilization of a HisTag fusion to the C-terminal end of the protein. A sample was extracted as above for activity assay. Purification was carried out following the manufacturer's protocol for gravity purification with the exception that elution was performed in 250 mM Imidazole instead of 500 mM (Pharmacia, HisTrap Column, 1.0 mL). Gel filtration was carried out on a PD-10 columns following manufacture's directions (Pharmacia) to exchange buffer from the high imidazole concentration of the Histag purified samples into 30 mM Bis-Tris pH 6.5 0.5 mM EDTA, 0.1% CHAPS for activity assays and storage. The sample was subject to activity assay as described above as well as analysis by SDS-PAGE.

### Example 16

### Transformation Vector Construction

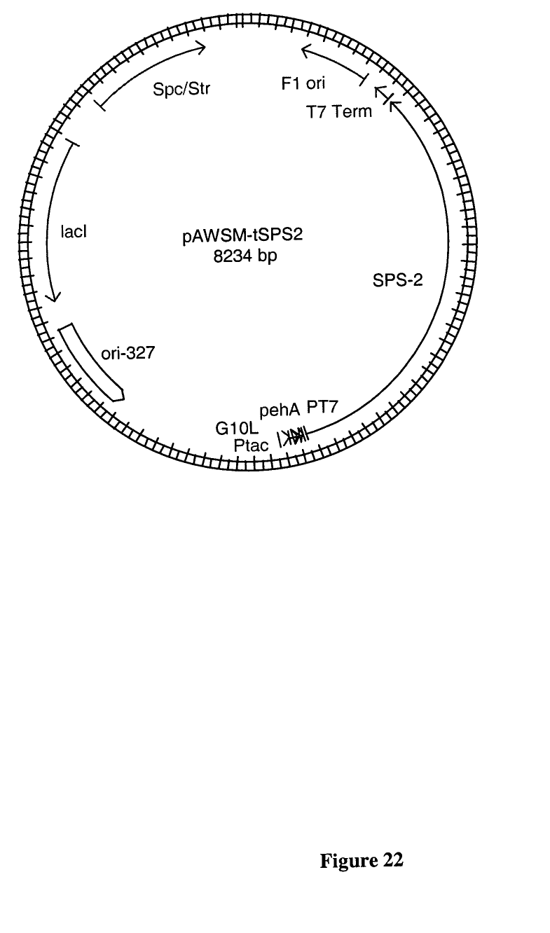

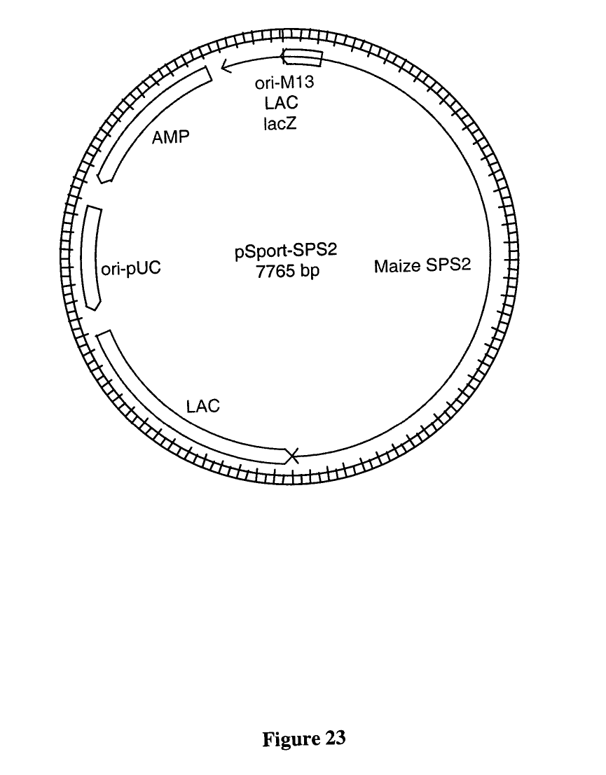

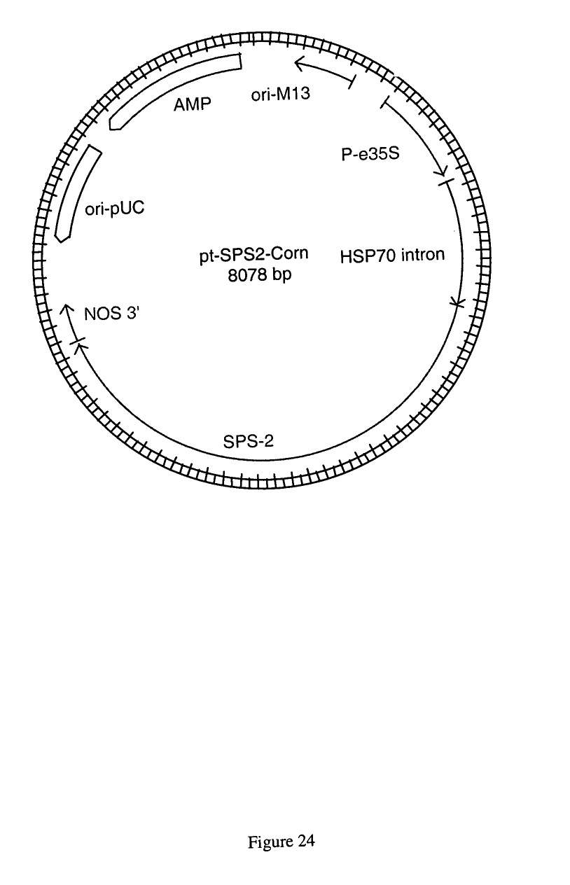

Transformation vectors could be made using any form of the SPS2 gene. For example the t-SPS2 gene could be subcloned from pMON52915 by excising the NcoI BamHI fragment, gel purification and subcloning into pMON13912 digested with the same enzymes to produce pt-SPS2-corn construct (FIG. 22). This construct can be used to produce, for example, a construct to contain the HSP 70 intron and 35S promoter (FIG. 23). Furthermore a similar procedure utilizing either additional PCR based on specific primers designed with these sequences and/or subcloning could be used to insert any form of this SPS2 gene, for example, a full-length, a truncated or a mutated SPS2 gene sequence, behind specific promoter and intron combinations (FIG. 24). Examples of the promoters to be used include PPDK and CAB (chlorophyll A/B binding protein) or PPDK promoter alone for leaf mesophyll cell expression, and the 35S and e35S—SSP promoters for maize protoplast transformation.

### Example 17

### Preparation and Transfection of Corn Leaf Protoplasts

All chemicals used in the following experiments are obtained from Sigma Chemical Company (St. Louis, Mo.) except as indicated. Corn leaf protoplast isolation is performed using modifications to the protocol of Sheen et al. (Plant Cell 3: 225-245, 1991). Seeds (Fr27 X FrMO17 from Illinois Foundation Seeds) are sterilized in a 500 ml sterile Corning storage bottle, polystyrene with a plug seal cap. Sterilization is performed by covering the seeds with 95-100% ethanol for 2 min. The seeds are then rinsed twice with sterile distilled water. Two drops of Tween 20 are added to the bottle, and the seeds are then covered with 50% Clorox® bleach (sodium hypochlorite) and allowed to sit for 30 min. The seeds are then rinsed four times with sterile distilled water, treated with 0.25 tsp Orthocide® (Captan Garden Fungicide, Chevron Chemical Co., San Ramon, Calif.) and 1 tsp Benlate® (50% benomyl, 50% inert ingredients; E.I. du Pont de Nemours and Company Agricultural Products, Wilmington, Del.), covered with sterile distilled water, and allowed to sit for 5 min.

Seedlings are germinated, 8 per Phytatray II™, on ½ MS medium (2.2 g/L MS Basal Salts (M-5524), 2.5 g/L Phytagel™) at approximately 80 mL per Phytatray II™. The seedlings are germinated embryo side down for 5 days in the light (incubator at 26° C. with a 16 hr day/8 hr night cycle under cool white fluorescent bulbs, 10-25 μE) followed by 7 to 8 days in the dark (26-28° C.). The procedure by Sheen et al. (The Plant Cell 3: 225-245, 1991) is modified for the use of completely etiolated tissue by omitting the final light treatment from the seed germination portion of the protocol.

After germination, the second true leaf (third emergent structure) is used for subsequent experimentation. The tips of the second true leaves are removed and the remainder cut into pieces that readily fit into 100 mm×25 mm petri dishes. The tissue is then wounded with a triple-bladed scalpel parallel to the direction of growth.

Wounded tissue is then placed in about 40 mL of enzyme mix (1% cellulase RS (Yakult Pharmaceutical, Tokyo, Japan; or Karlan, Santa Rosa, Calif.), 0.1% macerozyme (Yakult Pharmaceutical or Karlan), 0.6 M mannitol, 10 mM MES (2-[N-morpholino] ethanesulfonic acid), 1 mM CaCl₂, 1 mM MgCl₂, 0.1% bovine serum albumin, and 17 mM Beta-mercaptoethanol, pH 5.7). Seven to eight grams of leaf tissue is used per 40 mL of enzyme digestion media for a total of 4 separate enzyme digests. Digestion is performed in the light (cool white fluorescent bulbs, 10-25 μE) for 135 min at 50 rpm on an Orbit™ platform shaker at 26° C. After digestion, plates are swirled by hand at about 100 rpm for 50 seconds to release protoplasts from the tissue mass. Protoplasts are separated out by straining the enzyme mix through a 190 μm sieve, transferred to a 50 mL conical bottom centrifuge tube, and pelleted by centrifugation at 200×g for 8 min. The pellet is resuspended in 10 mL 0.6 M mannitol and centrifuged again at 200×g for 8 min.

The pellet is then resuspended in 10 mL of electroporation buffer (0.6 M mannitol, 4 mM MES (pH 5.7), 1.0 mM Beta-mercaptoethanol, 25 mM KCl, pH 5.7), the four tubes are pooled together and the cells are counted with a Hausser Scientific Bright-Line™ hemacytometer. Typical yields are 3-4×10⁶ protoplasts/g fresh weight of tissue. The protoplasts are then pelleted again and resuspended in electroporation buffer at a density of 4.5×10⁶ cells/mL.

In preparation for transfection with a plasmid of interest, 750 μl of protoplasts at 4.5×10⁶ cells/mL are added to each BioRad Gene Pulser® cuvette (0.4 cm gap) followed by the addition of DNA. Transfection is performed by electroporation at 125 μF and 260 V on a BioRad Gene Pulser™ Model No. 1652076, BioRad Capacitance Extender Model No. 1652087. Prior to and post transfection the cuvettes are placed on ice for 10 minutes. After being on ice for 10 minutes prior to electroporation the protoplasts and DNA are mixed by inverting the cuvettes twice.

After transfection, protoplasts are cultured overnight in agarose layered plates (MS Fromm+0.6 M mannitol+15 g/L SeaPlaque® agarose (FMC® Bioproducts)) in 7 mL of MS Fromm+0.6 M mannitol (4.4 g/L MS salts (Gibco, 500-1117EH), 1 mL/L 1000× vitamins (1.3 g/L nicotinic acid, 250 mg/L thiamine HCl, 250 mg/L pyridoxine HCl, 250 mg/L calcium panthothenate), 20 g/L sucrose, 2 mg/L 2,4-D, 0.1 g/L inositol (myo-inositol), 0.13 g/L asparagine, 109 g/L mannitol). This overnight culture is performed in an incubator at 26° C. with a 16 hr day/8 hr night cycle utilizing cool white fluorescent bulbs, 10-25 μE.

Protoplasts are harvested after one day; culture time was 18-22 hr. Protoplasts are removed from the plate using a 10 mL serological pipette, with care taken not to draw up the agarose layering. Protoplasts are then put in 15 mL conical bottom centrifuge tubes and centrifuged at 200×g for 8 min. The supernatant is removed and the pellets are placed immediately on dry ice. All pellets are then stored in a −80° C. freezer until assayed.

### Example 18

### Plant Transformation and Regeneration

Agrobacterium Induction and Inoculation

Agrobacterium tumefaciens (ABI strain) is grown in LB liquid medium (50 ml medium per 250 ml flask) containing 100 mg/L kanamycin and 50 mg/L spectinomycin for an initial overnight propagation (on a rotary shaker at 150 to 160 rpm) at 27° C. Ten ml of the overnight Agro suspension is transferred to 50 ml of fresh LB in a 250 ml flask (same medium additives and culture conditions as stated above) and is grown for approximately 8 hours. Suspension is centrifuged around 3500 rpm and pellet resuspended in AB minimal medium (now containing ½ the level of spectinomycin and kanamycin used for LB) containing 100 uM acetosyringone (AS, used for the induction of virulence) so a final concentration was 0.2×10⁹ cfu/mL (or an OD of 0.2 at 660 nm). These Agro cultures are allowed to incubate as described above for approximately 15 to 16 hours. The Agrobacterium suspension is harvested via centrifugation and washed in ½ MS VI medium containing AS. The suspension is then centrifuged again before being brought up in the appropriate amount of ½ MS PL (also containing AS) so that the final concentration of Agrobacterium is 1×10⁹ cfu/ml (which is equal to an OD of 1.0 at 660 nm).

Corn plant tissue pieces are put into a 1.5-ml Eppendorf tube with ½ MS PL containing Agrobacterium at an OD of 1.0. The eppendorf tube is capped tight and inverted a few times so that the tissue pieces are mixed well with the Agrobacterium suspension solution. The solution is poured into 2-3 layers of sterile Baxter filter paper (5.5 cm in diameter). The tissue pieces are removed from the filter paper by flipping the filter paper over and slightly pressing it against the co-culture medium in the petri dish. The ½ MS co-culture medium contains 3.0 mg/L 2,4-D, 200 uM acetosyringone, 2% sucrose, 1% glucose, 115 mg/L proline and 20 uM silver nitrate. The tissues are cultured at 23 C for 1 day and then are transferred to the first selection medium.

Regeneration

Paromomycin resistant callus is first moved to MS/6BA medium (crn 178) for 5 to 7 days. One to four pieces of callus is put in one plate. The medium contains essentially the same ingredients as selection medium except with 3.5 mg/l BA. After 6 BA pulse, callus with green shoot tips are moved to MSOD/P100 (crn 201) plate and are cultured for another 10 to 12 days. Usually 1 to 3 events are placed in one plate. The medium contained the following special ingredients: 0.3 g/l 1-asparagine, 0.2 g/l myo-inositol, 40 g/L maltose and 20 g/L glucose. Sucrose is replaced by maltose and glucose. This is the same medium as used in phytatray. After this stage, green shoots starts to grow out as well as white roots. Those small plantlets are transferred to phytatray (1 event per phytatray). After 2 to 3 weeks, as plantlets reach to the top of the lid inside the phytatray, plants are ready to be transplanted into soil. Usually 3 plants are selected from each event to be transplanted to soil. Plants are acclimated in the growth chamber for 1 week and then moved to greenhouse for hardening.

### Example 19

### Sucrose and Starch Measurements

A. The basic principle

1) Extract soluble sugars in hot water and analyze for glucose, fructose, and sucrose
2) Digest pellet with Amyloglucosidase (Sigma A-7255) and analyze supernatant for glucose to calculate starch content
   B. Sugar Extraction
1. Weigh 3-5 leaf punches (˜0.03-0.05 g per disc) into an eppendorf
2. Crush to a powder with a wooden applicator stick
3. Add 1 ml of 85 C water (the potato folks have a water bath)
4. Incubate at 85 C for 30 minutes
5. Spin in a microfuge for 10 minutes
6. Transfer supernatant to 15 ml conical on ice (this contains soluble sugars)
7. Add 1 ml of 85 C water to the pellet and repeat steps 4-6 for a total of 3 extractions. Combine all 3 supes (soluble sugars) in one 15 ml conical. Proceed to Glucose, Fructose, Sucrose microtiter assay.
   C. Starch Analysis
1. To the pelleted leaf tissue material (from Step 7 above) add 1 mL 0.2 N KOH. Vortex and incubate at 80° C. for 30 minutes. Also prepare a blank with no leaf tissue. (Use cap “locks”)
2. Add 250 μl of 0.5 M NaAcetate Buffer, pH 5.5 and 15 μl of Acetic Acid. Vortex well. (Make 15 ml per 50 samples, mix 15 ml of NaAc+0.9 ml Acetic acid, mix and add 250 per sample)
3. Add 20 Units of Amyloglucosidase in NaAcetate buffer (IU/μl is convenient, add 10 μl) and vortex again.
4. Incubate at 37° C. for 30 minutes.
5. Spin the tube at 3,000×g for 10 minutes in table top centrifuge.
6. Wash pellet 2× with 1 mL water. Combine the supernatant from each with the supe in Step 3.
7. Analyze for glucose content.
   D. Glucose, Fructose, Sucrose by Microtiter Plate Method (Using Boehringer Mannheim Enzyme Kits)
   Use following kit enzyme and buffers:
   Sucrose/D-Glucose/D-Fructose (Cat. No. 716 260)
   D-Glucose/D-Fructose (Cat. No. 139 106)
   Note that solutions in protocol refer to the Sucrose/Glucose/Fructose kit solutions
Final assay volume is 320 ul
Sample volumes for sucrose determination should be 10 ul
Sample volumes for glucose and fructose determination can range from 10 to 100 ul
   Step 1 (Sucrose Inversion)
For sucrose determination, sucrose is first inverted to glucose and fructose with B-fructosidase (invertase) and glucose is then determined
   Step 2 (Glucose Determination)
For glucose determination, glucose is phosphorylated with hexokinase to glucose-6-phosphate, which is then oxidized to gluconate-6-phosphate with glucose-6-phosphate dehydogenase. Hexokinase also phosphorylates fructose (to fructose-6-phosphate). The reduction of NADPH is measured at 340 nm.
   Step 3 (Fructose Determination)
For fructose determination, fructose-6-phosphate is isomerized to glucose-6-phosphate, which is then oxidized by glucose-6-phosphate dehydrogenase.
   Sucrose Determination
   Bring Solutions 1 and 2 to 25 C Before Use
   Aliquot Samples
(1) 10 μl samples per well (can do 40 samples in duplicate per plate)
   Note: can use up to 20 μl for sucrose
      Invert Sucrose
(2) 20 μl Solution 1 (B-fructosidase, pH 4.6)
   Mix on vortex (protect bottom of plate from vortex with its lid)
   Incubate 15 min at 25 C
      Assay Buffer
(3) 100 μl Solution 2 (buffer, pH 7.6, NADP, ATP) to all sample wells (10 ml per plate)
(4) add 170 μl H2O to all wells (bring to final volume of 300 μl)
(5) Preread absorbance (340 nm) on plate reader with automix on (to preread, go into setup, details and check the “preread” box)
   Glucose Determination
(6) Dilute Solution 3 (hexokinase) 1:7:
   1 plate: 150 μl Solution 3+1050 μl H2O
   2 plates: 300 μl Solution 3+2100 μl H2O
   3 plates: 450 μl Solution 3+3150 μl H2O
(7) 10 μl diluted Solution 3 to all wells
   Automix
   Incubate 15 min at 25 C
(8) Read absorbance (340 nm) on plate reader
   Glucose and Fructose Determination
   Aliquot Samples
(1) 30 μl samples to sample wells and standards (see end)
   Note: can use up to 100 μl for glucose, fructose
      Assay Buffer
(2) 100 μl Solution 2 (buffer, pH 7.6, NADP, ATP) to all sample wells (9700 μl per plate)
(3) add 170 μl H2O to all wells (bring to final volume of 300 μl)
(4) Preread absorbance (340 nm) on plate reader with automix on (to preread, go into setup, details and check the “preread” box)
   Glucose Determination
(5) Dilute Solution 3 (hexokinase) 1:7:
   1 plate: 125 μl Solution 3+875 μl H2O
   2 plates: 250 μl Solution 3+1750 μl H2O
(6) 10 μl diluted Solution 3 to all wells
   Automix
   Incubate 15 min at 25 C
(7) Read absorbance (340 nm) on plate reader
   Fructose Determination
(8) Dilute Solution 4 (phosphogluco isomerase) 1:7:
   1 plate: 150 μl Solution 3+1050 μl H2O
   2 plates: 300 μl Solution 3+2100 μl H2O
   3 plates: 450 μl Solution 3+3150 μl H2O
(9) 10 μl diluted Solution 4 to each well
   Automix
   Incubate 15 min at 25 C
(10) Read absorbance (340 nm) on plate reader

Standard Curves:

Aliquot 10 μl of standard (Standard curve 0.1 to 8 μg)

| Final | Stock | |
|---|---|---|
| μg/μl | (1 μg/μl) | H20 |
| 0.8 | 800 | 200 |
| 0.4 | 400 | 600 |
| 0.2 | 200 | 800 |
| 0.1 | 100 | 900 |
| 0.08 | 80 | 920 |
| 0.04 | 40 | 960 |
| 0.02 | 20 | 980 |
| 0.01 | 10 | 990 |

### Example 20

### SPS Activity in Transgenic Maize

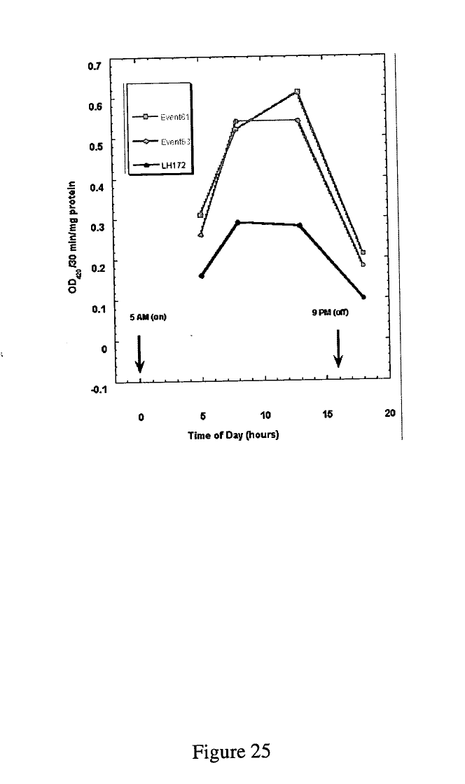

SPS samples were measured from some of the maize leaf samples from the ppdk-Δ469 events (corn plants containing this construct are called Pat; the construct contains a truncated SPS being driven by the ppdk promoter) at various time points throughout the day. The samples from which the activity assays were performed were exactly the same samples, which had been analyzed by protein immunoblots. The band for the transgenic truncated maize SPS was present in the samples when they were analyzed by protein immunoblots. FIG. 25 shows the activity of leaf SPS from two plants positive for the SPS transgene as well as wild type (LH172) plants. FIG. 25 shows that the transgenic maize plants had considerably higher SPS activity through out the diurnal cycle, but the increase in SPS activity was especially great during the middle of the light period (1 PM and 5 PM).

### Example 21

### SPS Greenhouse Efficacy Experiment

Several experiments were performed to test the source efficacy of ppdk-Δ469 and CAB-Δ469 (corn events containing this construct are called Zeke; the construct contains a truncated SPS being driven by the CAB promoter) SPS events in inbred (LH172) maize grown in the greenhouse. While these experiments varied in some of the details the basic plan of all of these experiments was always the same. F1 seed was planted in trays and then transferred to 6″ pots at the V1 or V2 stage. PCR assays were performed to identify plants positive and negative for the transgene. All plants from a single event were blocked together and surrounded by LH 172 wild type plants to prevent border effects. Between V6 and V10 the entire uppermost fully expanded leaf was sampled at several time points through out the day. Each plant was sampled only once so the sample size varied with the number of plants from each event. Plants which different phenotypically or visually from the average were not included in the study.

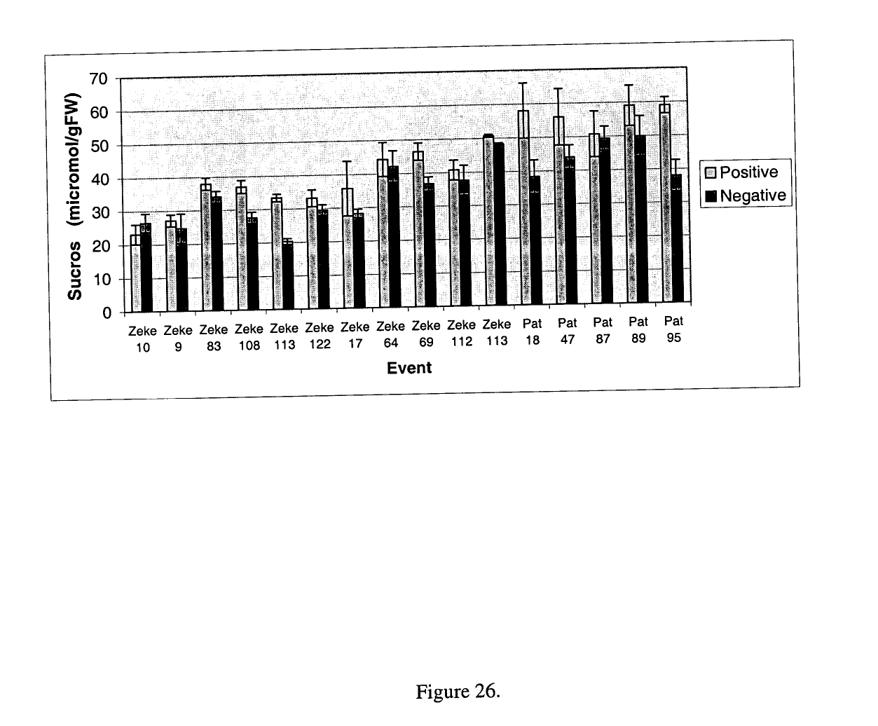

All of the ppdk-Δ469 and CAB-Δ469 SPS events tested had a trend toward higher steady state sucrose in the afternoon (positive vs. negative comparison); in the majority of these events the higher sucrose levels in the positive plants compared to the negative plants was statistically significant (FIG. 26). FIG. 26 shows the sucrose levels for all the events tested at 6 PM. This data comes from several different similar experiments. The greatest increase in sucrose was 60% in Pat 18 (FIG. 26). Most of these events also showed a trend toward decreased leaf starch levels but this change in starch levels was not as consistent (across all the events) as the increase in sucrose levels. Many of these events also had increased sucrose levels at other time points during the day. However, the 6 PM time point was the only time point in which all events showed the trend toward increased sucrose. In addition at least for the ppdk-Δ469 events the trend toward higher sucrose was greatest at 6 PM.

### Example 22

### Expression of Transgenic Maize SPS in Hybrid Maize

The next step in testing the SPS transgene was to make hybrids by crossing the LH172 plants expressing the truncated SPS with LH244 tester plants to make hybrids. It was necessary to confirm that these hybrids still expressed the truncated SPS in their leaves.

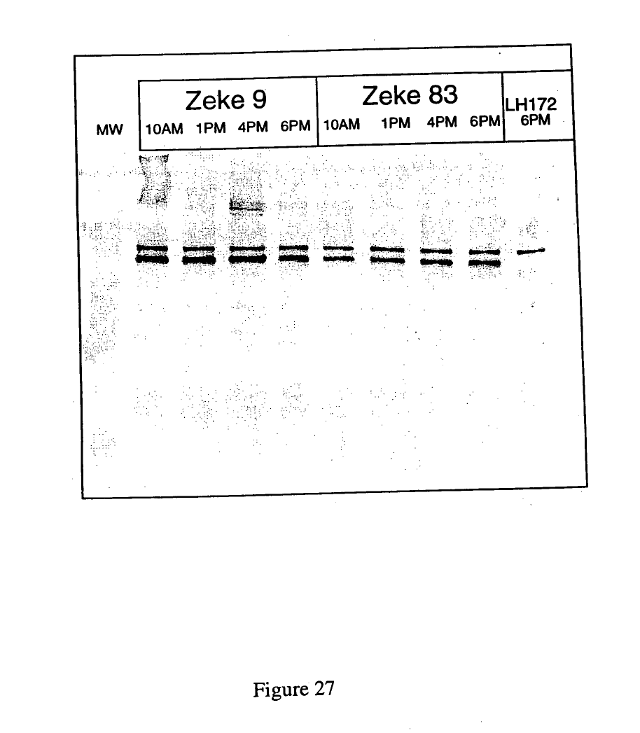

Western Blot analysis of leaf samples from selected field-grown hybrid plants proves that Δ469 SPS protein accumulates in maize leaves throughout the day (FIG. 27.)

### Example 23

### Field Efficacy Experiments

Since the truncated SPS was expressed in maize leaves of hybrid plants, five field experiments were performed to test the efficacy of the SPS transgene. They include 4 one-location experiments to test source efficacy and a six-location experiment to test yield and yield components (to be described in the next section). Of the four source efficacy experiments three of the source efficacy experiments were performed on hybrids made by crossing LH172 plants homozygous for maize SPS with LH244 testers. The final experiment was performed using inbred maize plants homozygous for the SPS transgene. The source efficacy experiment consists of comparing the steady-state sucrose, starch, fructose and glucose levels in plants positive or negative for the transgene. Other than the fact that these experiments were performed on hybrids in the field the plan of the experiment was very similar to those previously performed in the greenhouse.

In the three hybrid experiments several ppdk-Δ469 SPS events and CAB-Δ469 SPS events overexpressing SPS were tested. We also tested 2 selections each of 2 CAB-Δ469 SPS events, which were cosuppressed for the SPS gene (both endogenous SPS and transgenic SPS are not visible on Western Blots).

Source at V8

In the first experiment, hybrid maize plants positive or negative for Δ469 SPS were sampled at various time points throughout two consecutive days. 6 ppdk-Δ469 SPS events and 6 CAB-Δ469 SPS events were tested. They represent a range of expression levels for Δ469 SPS. We sampled the entire uppermost fully expanded leaf. The sample size was 8 (statistically n=8). The first day we sampled at four-time points (9 AM, 1 PM, 3 PM and 5 PM). On the second day a separate set of plants were sampled at 1 PM, 6 PM and 7 PM. The second separate set of plants was originally intended to act as insurance in case of the plants were damaged by weather. Each plant was sampled only once. From previous non-transgenic experiments we calculated that we should be able to resolve an 8-10% increase in sucrose (p=0.1). Table 10 depicts a generalized map of one of the two sets of plants used for this experiment.

TABLE 10
Plan for V8 Source Efficacy Experiment in field.

| | Range 1 | Range 2 | Range 3 | Range 4 | Range 5 | Range 6 | Range 7 | Range 8 | Range 9 |
|---|---|---|---|---|---|---|---|---|---|
| Row 1 | LH172 × LH244 | LH172 × | LH172 × | LH172 × LH244 | LH172 × | LH172 × LH244 | LH172 × | LH172 × | LH172 × LH244 |
| | | LH244 | LH244 | | LH244 | | LH244 | LH244 | |
| Row 2 | LH172 × LH244 | PatE1+ | PatE3+ | LH172 × LH244 | PatE5+ | ZekeE1+ | ZekeE3+ | ZekeE5+ | LH172 × LH244 |
| Row 3 | LH172 × LH244 | PatE1+ | PatE3+ | LH172 × LH244 | PatE5+ | ZekeE1+ | ZekeE3+ | ZekeE5+ | LH172 × LH244 |
| Row 4 | LH172 × LH244 | PatE1− | PatE3− | LH172 × LH244 | PatE5− | ZekeE1− | ZekeE3− | ZekeE5− | LH172 × LH244 |
| Row 5 | LH172 × LH244 | PatE1− | PatE3− | LH172 × LH244 | PatE5− | ZekeE1− | ZekeE3− | ZekeE5− | LH172 × LH244 |
| Row 6 | LH172 × LH244 | PatE2+ | PatE4+ | PatE6+ | ZekeE2+ | LH172 × LH244 | ZekeE4+ | ZekeE6+ | LH172 × LH244 |
| Row 7 | LH172 × LH244 | PatE2+ | PatE4+ | PatE6+ | ZekeE2+ | LH172 × LH244 | ZekeE4+ | ZekeE6+ | LH172 × LH244 |
| Row 8 | LH172 × LH244 | PatE2− | PatE4− | PatE6− | ZekeE2− | LH172 × LH244 | ZekeE4− | ZekeE6− | LH172 × LH244 |
| Row 9 | LH172 × LH244 | PatE2− | PatE4− | PatE6− | ZekeE2− | LH172 × LH244 | ZekeE4− | ZekeE6− | LH172 × LH244 |
| Row 10 | LH172 × LH244 | LH172 × | LH172 × | LH172 × LH244 | LH172 × | LH172 × LH244 | LH172 × | LH172 × | LH172 × LH244 |
| | | LH244 | LH244 | | LH244 | | LH244 | LH244 | |

We expect that all the events should show a trend toward increase in sucrose (positive vs. negative) at the later-day time points. Since we should be able to resolve a 10% increase in sucrose, statistically it is probable that the majority of these events will show a statistically significant increase in steady-state sucrose levels at the 5 PM and 6 PM time points. A general trend toward decreasing starch may also be observed but this may not be as consistent as the sucrose results. This result will confirm that the over-expressing SPS in maize will increase source capacity in hybrid maize at around the same levels as we previously observed in inbred maize (10-60%) and prove that SPS is the rate-limiting enzyme for sucrose production in hybrid maize.

Source Capacity in Co-Suppressed Events

It was observed earlier in inbred maize that certain events not only did not express the SPS transgene but also were also deficient in leaf SPS protein and activity. Since these plants are experiencing lower SPS levels and SPS is thought to be the crucial enzyme of sucrose synthesis it was of interest to observe the effects of cosuppressing leaf SPS on the plants growth, development and sucrose levels. Early experiments with inbred maize using plants completely deficient in leaf SPS activity suggested that the co-suppression of SPS leads to decreased leaf sucrose and increased leaf starch the opposite of what is observed in the overexpressing events. None of these effects was shown to be statistically significant and interestingly no obvious changes in plant growth, size or phenotype were seen.

In two events (Zeke 10 and Zeke59) SPS protein and activity was almost completely eliminated. Hybrid plants (LH172×LH244) from 2 positive and 2 negative selections for both of these events were tested for phenotype and source capacity. The field plan for this experiment is shown in Table 11. We have not yet proven that these hybrid plants have lower levels of SPS activity or protein although these studies would be done along with any further work.

TABLE 11
Field Plant for V8 Co-suppressed Efficacy Experiment at Jerseyville

| | | | | | | | | |
|---|---|---|---|---|---|---|---|---|
| LH172 × LH244 | LH172 × | LH172 × | LH172 × | LH172 × | LH172 × LH244 | LH172 × LH244 | LH172 × LH244 | LH172 × LH244 |
| | LH244 | LH244 | LH244 | LH244 | | | | |
| LH172 × LH244 | E1+ | E2+ | E3+ | E4+ | LH172 × LH244 | LH172 × LH244 | LH172 × LH244 | LH172 × LH244 |
| LH172 × LH244 | E1+ | E2+ | E3+ | E4+ | LH172 × LH244 | LH172 × LH244 | LH172 × LH244 | LH172 × LH244 |
| LH172 × LH244 | E1− | E2− | E3− | E4− | LH172 × LH244 | LH172 × LH244 | LH172 × LH244 | LH172 × LH244 |
| LH172 × LH244 | E1− | E2− | E3− | E4− | LH172 × LH244 | LH172 × LH244 | LH172 × LH244 | LH172 × LH244 |
| LH172 × LH244 | LH172 × | LH172 × | LH172 × | LH172 × | LH172 × LH244 | LH172 × LH244 | LH172 × LH244 | LH172 × LH244 |
| | LH244 | LH244 | LH244 | LH244 | | | | |

We sampled the entire uppermost fully expanded leaf at 9 AM, 1 PM, 3 PM and 5 PM. The sample size was 8 and each plant was sampled only once. There were no apparent differences in phenotypes between positives and negatives.

Limited previous results using co-suppressed inbred SPS events suggested that co-suppression of SPS would lead to higher levels of leaf starch and lower levels of leaf sucrose which is just opposite of what we observed when the gene is over-expressed. We expect a similar result in these hybrid plants, however the magnitude of the response might be greater since the hybrid plants are presumably more optimized with respect to source capacity. Just as we observed with the inbred co-suppressed maize no changes in plant size, growth rate or phenotype were observed.

Source Capacity at Kernel Fill in Hybrid Maize

Since all previous experiments had looked at the source capacity in early vegetative-stage maize (e.g. V6 to V10) it is of interest to know whether or not the transgene can increase source capacity at kernel fill when yield is being determined. In order to test this the same 12 events that were tested at V8 (see section A) were also tested at about 20 days after pollination. This is during early kernel fill when source requirements are at a maximum. It was also of interest to determine the effect of density on any potential source effect of the SPS transgene and therefore rows of plants from each event were thinned to two densities—a normal planting density (25,000 plants/acre) and a higher planting density (37,000 plants/acre). The higher density was calculated to reduce overall grain yield since it would be stressful to the plants. A generalized field map for this experiment is shown in Table 12.

TABLE 12
Field Map for Kernel Fill Source Efficacy Experiment at a Single Density

| | Range 1 | Range 2 | Range 3 | Range 4 | Range 5 | Range 6 | Range 7 | Range 8 |
|---|---|---|---|---|---|---|---|---|
| Row 1 | LH172 × LH244 | LH172 × | LH172 × LH244 | LH172 × | LH172 × LH244 | LH172 × LH244 | LH172 × LH244 | LH172 × LH244 |
| | | LH244 | | LH244 | | | | |
| Row 2 | LH172 × LH244 | PatE1+ | LH172 × LH244 | PatE3+ | PatE5+ | ZekeE1+ | ZekeE3+ | ZekeE5+ |
| Row 3 | LH172 × LH244 | PatE1− | LH172 × LH244 | PatE3− | PatE5− | ZekeE1− | ZekeE3− | ZekeE5− |
| Row 4 | LH172 × LH244 | PatE2+ | LH172 × LH244 | PatE4+ | PatE6+ | ZekeE2+ | ZekeE4+ | ZekeE6+ |
| Row 5 | LH172 × LH244 | PatE2− | LH172 × LH244 | PatE4− | PatE6− | ZekeE2− | ZekeE4− | ZekeE6− |
| Row 6 | LH172 × LH244 | PatE1+ | PatE3+ | PatE5+ | ZekeE1+ | ZekeE3+ | LH172 × LH244 | ZekeE5+ |
| Row 7 | LH172 × LH244 | PatE1− | PatE3− | PatE5− | ZekeE1− | ZekeE3− | LH172 × LH244 | ZekeE5− |
| Row 8 | LH172 × LH244 | PatE2+ | PatE4+ | PatE6+ | ZekeE2+ | ZekeE4+ | LH172 × LH244 | ZekeE6+ |

We harvested the entire leaf one node above the ear leaf. Based on the previous results, we expect to see increased levels of SPS activity and source capacity during kernel fill which means we should see significant increases in steady-state leaf sucrose levels and potentially decreases in steady state leaf starch levels in these plants. Given that these transgenic plants appear to perform somewhat more poorly at higher density with respect the yield (please see data below) we might expect to see that in terms of source the gene also performs more poorly at the higher density.

Source Effect of SPS on Field Grown Inbreds

Since it is possible that the previous three field experiments would not show that the effect of SPS transgene on greenhouse grown maize translates to hybrid field-grown maize a bridging experiment was performed to look at the effect of the over-expression of maize SPS on field grown inbred maize. This experiment should allow us to separate the two parameter changes in the previous experiment (greenhouse→field and inbred→hybrid)

6 ppdk-Δ469 SPS events and 6 CAB-Δ469 were tested with comparisons between homozygous positive and negative plants. Some but not all of these events were the same as were tested in the hybrid maize (Sections A and C). The map for the inbred efficacy trial is shown in Table 13. This experiment was harvest when the plants were at the V8 stage. We had planned to take samples at 3 time points (1 PM, 4 PM and 6 PM) however; poor germination reduced the number of plants we could harvest. Therefore, most events had leaf samples harvested from only one or two time points.

TABLE 13
Field Map for Inbred Efficacy Trial Including ppdk- E. coli FDAII events

| | Range 1 | Range 2 | Range 3 | Range 4 | Range 5 | Range 6 | Range 7 | Range 8 | Range 9 |
|---|---|---|---|---|---|---|---|---|---|
| Row 1 | LH172 × LH244 | LH172 × | LH172 × | LH172 × | LH172 × | LH172 × | LH172 × | LH172 × | LH172 × |
| | | LH244 | LH244 | LH244 | LH244 | LH244 | LH244 | LH244 | LH244 |
| Row 2 | LH172 × LH244 | LH172 | LH172 | LH172 | LH172 | LH172 | LH172 | LH172 | LH172 |
| Row 3 | LH172 × LH244 | LH172 | PatE1+ | PatE3+ | PatE5+ | ZekeE2+ | ZekeE4+ | ZekeE6+ | LH172 |
| Row 4 | LH172 × LH244 | LH172 | PatE1+ | PatE3+ | PatE5+ | ZekeE2+ | ZekeE4+ | ZekeE6+ | LH172 |
| Row 5 | LH172 × LH244 | LH172 | PatE1− | PatE3− | PatE5− | ZekeE2− | ZekeE4− | ZekeE6− | LH172 |
| Row 6 | LH172 × LH244 | LH172 | PatE1− | PatE3− | PatE5− | ZekeE2− | ZekeE4− | ZekeE6− | LH172 |
| Row 7 | LH172 × LH244 | LH172 | PatE2+ | PatE4+ | PatE6+ | FredE1+ | FredE3+ | FredE5+ | LH172 |
| Row 8 | LH172 × LH244 | LH172 | PatE2+ | PatE4+ | PatE6+ | FredE1+ | FredE3+ | FredE5+ | LH172 |
| Row 9 | LH172 × LH244 | LH172 | PatE2− | PatE4− | PatE6− | FredE1− | FredE3− | FredE5− | LH172 |
| Row 10 | LH172 × LH244 | LH172 | PatE2− | PatE4− | PatE6− | FredE1− | FredE3− | FredE5− | LH172 |
| Row 11 | LH172 × LH244 | LH172 | ZekeE1+ | ZekeE3+ | ZekeE5+ | FredE2+ | FredE4+ | FredE6+ | LH172 |
| Row 12 | LH172 × LH244 | LH172 | ZekeE1+ | ZekeE3+ | ZekeE5+ | FredE2+ | FredE4+ | FredE6+ | LH172 |
| Row 13 | LH172 × LH244 | LH172 | ZekeE1− | ZekeE3− | ZekeE5− | FredE2− | FredE4− | FredE6− | LH172 |
| Row 14 | LH172 × LH244 | LH172 | ZekeE1− | ZekeE3− | ZekeE5− | FredE2− | FredE4− | FredE6− | LH172 |
| Row 15 | LH172 × LH244 | LH172 | LH172 | LH172 | LH172 | LH172 | LH172 | LH172 | LH172 |
| Row 16 | LH172 × LH244 | LH172 × | LH172 × | LH172 × | LH172 × | LH172 × | LH172 × | LH172 × | LH172 × |
| | | LH244 | LH244 | LH244 | LH244 | LH244 | LH244 | LH244 | LH244 |

We expect to observed increases in steady state sucrose levels and decreases in steady state starch levels at the afternoon time points (especially 4 and 6 PM) in all SPS events in this experiment. If this source efficacy is observed in the hybrid experiments (Sections A and C) we may choose not to analyze these samples.

### Example 24

### Yield and Yield Components Trial

A six-location yield trial was designed to test the effect of SPS on yield, yield components, agronomics and kernel chemistry (proximate analysis). At this point the only data available is yield, kernel moisture and plant height. The same 12 events, which were tested in the hybrid efficacy study, were used in the six-location yield trial. One positive and one negative selection was used for each event. Only a single hybrid (LH172×LH244) was used in the study. The experiment was performed at two separate densities (25,000 plants/acre and 37,000 plants/acre). It was calculated that this trial has the power to detect a 15% increase in yield at p<0.1 of 82%. Results were analyzed in two ways. In the first case, positive and negative comparisons were made for each event across densities and in the second case, positive and negative comparisons were made for each event at each density.

Table 14 shows the yield results in which results were analyzed at both densities. From this figure it can be seen that the only significant effect on yield was that Pat 95 positives had 6.9% higher yield compared to the negatives at the normal planting density. At high density Pat 95 positives had a 4.3% higher yield (non significant) compared with the negatives. When the results were analyzed across densities the only significant yield effect was that Pat 95 positives had a 5.6% increase in yield compared to the negatives.

Kernel water content and plant height was also analyzed in this study. Zeke 112 had significantly higher kernel water content at high density and across densities while Pat 87 plants were significantly larger both at high density and across densities.

Overall the conclusion from all of this data is that over-expression of maize SPS in maize leads to an increase in source capacity. In certain cases this increase in source capacity can translate into higher grain yields, larger plants or seed with higher water content.

TABLE 14
Yields Differentials of Hybrid Maize Overexpressing SPS

| | | | | POS | |
|---|---|---|---|---|---|
| | | | | minus | |
| Density | Event | EST_POS | EST_NEG | NEG | PVALUE |
| High | Pat018 | 167.78 | 174.65 | −6.87 | 0.3396 |
| High | Pat066 | 173.62 | 178.1 | −4.49 | 0.4954 |
| High | Pat085 | 187.6 | 183.09 | 4.51 | 0.4933 |
| High | Pat087 | 176.91 | 184.04 | −7.13 | 0.2791 |
| High | Pat089 | 173.28 | 174.62 | −1.34 | 0.8388 |
| High | Pat095 | 184.17 | 176.54 | 7.63 | 0.2474 |
| High | Zeke011 | 176.81 | 185.3 | −8.49 | 0.198 |
| High | Zeke017 | 180.93 | 179.72 | 1.21 | 0.8537 |
| High | Zeke019 | 183.13 | 181.82 | 1.31 | 0.842 |
| High | Zeke064 | 186.85 | 184.73 | 2.12 | 0.7471 |
| High | Zeke069 | 182.39 | 184.09 | −1.7 | 0.7958 |
| High | Zeke112 | 170.88 | 181.57 | −10.68 | 0.1062 |
| Low | Pat018 | 170.62 | 171.68 | −1.07 | 0.871 |
| Low | Pat066 | 165.39 | 171.53 | −6.15 | 0.3507 |
| Low | Pat085 | 167.76 | 174.01 | −6.25 | 0.3431 |
| Low | Pat087 | 167.55 | 165.72 | 1.83 | 0.7808 |
| Low | Pat089 | 162.63 | 170.5 | −7.87 | 0.233 |
| Low | Pat095 | 178.23 | 166.79 | 11.44 | 0.0839 |
| Low | Zeke011 | 165.7 | 167.33 | −1.63 | 0.8043 |
| Low | Zeke017 | 168.37 | 169.52 | −1.15 | 0.8613 |
| Low | Zeke019 | 171.26 | 167.65 | 3.61 | 0.5828 |
| Low | Zeke064 | 163.63 | 161.71 | 1.93 | 0.7697 |
| Low | Zeke069 | 164.7 | 166.66 | −1.96 | 0.7658 |
| Low | Zeke112 | 164 | 163.64 | 0.36 | 0.9563 |

### Example 25

### Sequences of 7 Maize SPS Genes

The prior examples show the analysis of one several SPS enzymes. We also include herein several other SPS enzymes we have discovered. These enzymes would also be expected to function in the present invention. Based on our results in the foregoing examples, we expect that causing heterologous expression of SPS in mesophyll of corn may be one important aspect of increasing source capacity in a plant which contains this tissue.

Based on all available evidence including public databases and Monsanto internal databases we have identified 7 unique SPS sequences from maize. 5 of these sequences are full length, and two are partial sequences (SEQ ID NOs: 59-71). In addition we have found the sequence denoted as SEQ ID NO:53 and its variants (SEQ ID Nos: 55 and 57).

Table 15 shows the tissue distribution of these sequences in maize (SEQ ID NOs: 59-71).

TABLE 15
EST distribution maize SPS sequences in different
tissues used to construct cDNA libraries.

| | Root | | Stem | | Leaf | | Ear | | Tassel | | Most | | | Total | |
|---|---|---|---|---|---|---|---|---|---|---|---|---|---|---|---|
| SPS | EST | % | EST | % | EST | % | EST | % | EST | % | Tissue | EST | % | EST | % |
| ZmSPS1F | 0 | 0.0 | 5 | 8.6 | 34 | 58.6 | 10 | 17.2 | 9 | 15.5 | Leaves | 34 | 58.6 | 58 | 100 |
| ZmSPS2F | 12 | 18.2 | 4 | 6.1 | 29 | 43.9 | 21 | 31.8 | 0 | 0.0 | Leaves | 29 | 43.9 | 66 | 100 |
| ZmSPS3F | 13 | 10.5 | 25 | 20.2 | 30 | 24.2 | 42 | 33.9 | 14 | 11.3 | Ear | 42 | 33.9 | 124 | 100 |
| ZmSPS4F | 5 | 17.2 | 3 | 10.3 | 10 | 34.5 | 11 | 37.9 | 0 | 0.0 | Ear | 11 | 37.9 | 29 | 100 |
| ZmSPS5F | 8 | 29.6 | 1 | 3.7 | 7 | 25.9 | 11 | 40.7 | 0 | 0.0 | Ear | 11 | 40.7 | 27 | 100 |
| ZmSPS6 | 0 | 0.0 | 0 | 0.0 | 0 | 0.0 | 12 | 92.3 | 1 | 7.7 | Ear | 12 | 92.3 | 13 | 100 |
| ZmSPS7 | 1 | 4.2 | 2 | 8.3 | 6 | 25.0 | 11 | 45.8 | 4 | 16.7 | Ear | 11 | 45.8 | 24 | 100 |

Each ZmSPS DNA sequences were used as queries to BLAST search Monsanto maize EST database.
Hit sequences from each search that has 97% or higher identity to the query sequences were taken as representative sequences of the query.
The cDNA library source of each of these hits were then traced and summarized in this table.

### Example 26

### Analysis of the Regulatory Sites of SPS

3.3. Regulatory Sites on Higher Plant SPS Proteins

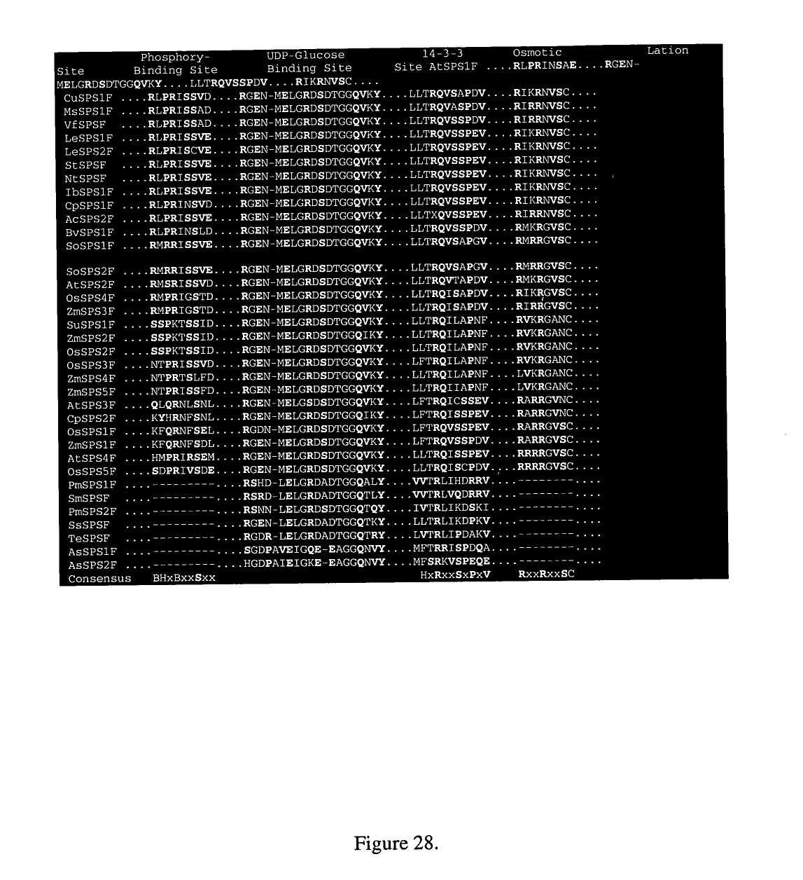

FIG. 28 shows a sequence alignment for four important regions in SPS including the UDP-glucose binding site, a 14-3-3 binding site and two regulatory phophorylation sites. A summary of these sites in each of the 7 groups of SPS is in Table 16. A tree has been developed to look at the evolutionary relationship between SPS enzymes. This analysis has shown that the SPS enzymes fall into 7 groups. It is important to note that most of the major regulatory sites that have been identified in the sequence of SPS are found in all the major higher plant SPS genes but not in the bacterial forms of the enzyme. An example of this includes the major light/dark phosphorylation site (Ser158 in spinach) which is found in all SPS proteins (FIG. 28) including those which do not appear to have an SPS which undergoes reversible phosphorylation in response to light/dark changes in the leaf (e.g. tomato). It has been reported that there is an isozyme in rice, which does not appear to undergo reversible phosphorylation. Phosphorylation of the enzyme at this site is inhibitory (3). All of the rice sequences in this report contain this regulatory phosphorylation site. A second phosphorylation site that is phosphorylated during osmotic stress (Ser 428 in spinach) is not found in all isoforms (FIG. 28) giving rise to the possibility that a distinct form of SPS in plants is regulated in response to stress. It has been shown that the SPS genes, which contain this site, come from all branches except branches of Group 3 and 5. Interestingly, members in Group 3 and 5 all lack this site. A third phosphorylation site (Ser 229) is suggested to be the site of interaction between SPS and a 14-3-3 protein, however the physiological significance of such site is not clear. This phosphorylation site is only found in some SPS proteins (FIG. 28) and seems to be missing in all members of Group 5. Two other phosphorylation sites Ser127 and Ser689 in spinach leaf SPS also exist but phosphorylation on these sites are not thought to be of regulatory significance. These two sites are also not universally found in all higher plant SPS proteins.

TABLE 16
Summary of some features of each SPS groups.

| | | | UDP- | | |
|---|---|---|---|---|---|
| | | | Glucose | 14-3-3 | Osmotic |
| | | Phosphorylation | Binding | Binding | Regulation |
| Group | Distribution | Site | Site | Ste | Site |
| 1 | Microbe | No | Yes | No | No |
| 2 | Dicot | Yes | Yes | Yes | Yes |
| 3 | Dicot | Yes | Yes | Yes | No |
| 4 | Monocot | Yes | Yes | Yes | Yes |
| 5 | Monocot | Yes | Yes | No | No |
| 6 | Monocot | Yes | Yes | Yes | Yes |
| 7 | Dicot and | Yes | Yes | Yes | Yes |
| | Monocot | | | | |

### Example 27

### Hypothesis for Cold and Drought Tolerance of SPS Transgenic Maize

Expression of transgenic SPS in maize leaves may result in plants with increased drought or cold tolerance.

Plant adaptation to low temperature stress often involves the accumulation of sucrose (Guy et al., Plant Physiology: 502-508, 1992). This increase in sucrose in both potato tubers (Geigenberger et al., The regulation of sucrose synthesis in leaves and tubers of potato plants. Sucrose Metabolism, Biochemistry, Physiology and Molecular Biology. Rockville, Md., American Society of Plant Physiologists, 1995) and photosynthetic tissues (Guy et al., Plant Physiology: 502-508, 1992) has been linked with an increase in SPS activity. In spinach leaves de novo synthesis of SPS was shown to be at least partially responsible for this increase in activity (Guy et al., Plant Physiology: 502-508, 1992) while in potato tubers the increase in activity correlated with the appearance of a new isoform of SPS (Reimholtz et al., Plant Cell Environ 20:291-305, 1997).

Leaf specific overexpression of maize SPS in tomato increased the oxygen sensitivity of photosynthesis. The temperature at which photosynthesis was no longer stimulated by low O₂ was decreased by an average of 3° C. in one transgenic line relative to the wild type (Laporte et al., Planta 212:817-822, 2001). This suggests that sucrose synthesis is limited by oxygen at low temperatures. Increasing the rate of sucrose synthesis under these conditions may result in enhanced growth at lower temperatures. Another study found that SPS activities increased 2-fold in Arabidopsis in the leaves of plants grown at 5° C. compared to 23° C. (Strand et al., Plant Physiol 199:1387-1397, 1999). Thus, the increase in activity of SPS may be part of a general response to cold stress.

When spinach leaves or potato tubers are incubated in hyperosmotic solutions to induce osmotic stress, activation of SPS occurs (Winter and Huber, Crit Rev in Plant Science 19:31, 2000). It is thought that this increase in activity results from the regulatory phosphorylation of the enzyme on Ser-424. This positive regulation may act by an antagonistic effect on the negative regulation by phosphorylation on Ser-158. It has been suggested that the kinase responsible for this phosphorylation may be involved in a drought stress response (Winter and Huber, Crit Rev in Plant Science 19:31, 2000).)

It is well known that the expression of a number of genes can be induced by both drought and cold stress even though these two stresses appear to be quite different (Liu et al. 1998). Therefore overexpression of SPS in maize may result in plants, which have increased sucrose production, cold tolerance drought tolerance and yield.

### Example 28

### Hypothesis for the Overexpression of SPP, FDA and UGPase

Evidence exists for an in vivo association between SPS and SPP in leaves (Echeverria et al., Plant Phys 115:223, 1997). This complex between SPS and other proteins will require additional efforts to allow us to manipulate this pathway in vivo. First, if most of the carbon is channeled through this pathway additional exogenous overexpressed SPS would have reduced access to its substrate. Second, associated proteins might activate, stabilize or promote the synthesis of SPS. In either event, coexpression of SPP along with SPS would allow the overexpressed proteins to associate in vivo under the same conditions that the endogenous enzymes would associate and therefore increase the flow of carbon through this pathway. It is expected that this should lead to further increases in sucrose production over and above that which is observed with the expression of SPS alone.

Another enzyme in the sucrose synthesis pathway is UDPglucose pyrophosphorylase. UDP glucose pyrophosphorylase is the first enzyme in the pathway and it provides substrate for SPS. Recent evidence suggests that these two enzymes are both 14-3-3 binding proteins and this association with 14-3-3 cements them together into a protein complex in vivo (Winter and Huber, Crit Rev in Plant Science 19:31, 2000). It is therefore possible that a complex consisting of all three enzymes exist in plants. If such a complex exists then all members of this complex are logical targets for increased sucrose biosynthesis. In plants UDPglucose has been cloned in barley (Eimert et al., Gene 170:227-232, 1996). Recent results in this laboratory suggest that UDPglucose pyrophosphorylase is either closely related to SPP or may in fact be the same protein.

SPS is regulated by reversible phosphorylation (Winter and Huber, Crit Rev in Plant Science, 19:31, 2000) and there is some evidence that it may be associated with a protein kinase (Huber and Huber, Biochem Biophys Acta 1091:393, 1991). It may be that association with this protein kinase is also necessary for optimal SPS activity, stability, and expression.

Thus a sucrose synthesizing complex similar to the pyruvate dehydrogenase complex may exist in plants. The co-overexpression of any or all of these enzymes along with SPS might provide additional sucrose production in leaves.

### Example 28

1. Construction of Vectors for Soybean Transformation

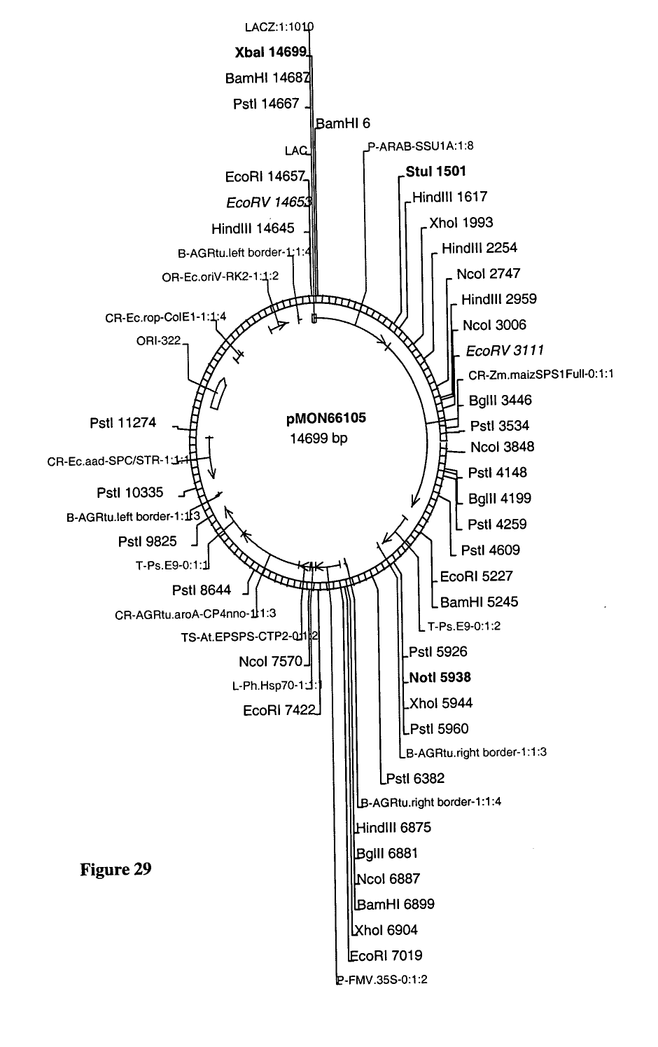

A binary vector, pMON66105 (FIG. 29), was made for over-expressing maize SPS 1 gene (SEQ ID NO: 53) in soybean under leaf specific promoter SSU. PMON66105 is a 2 T-DNA vector, where the selectable marker expression cassette [P-FMV/HSP70/CTP2/CP4/E9] and the SPS 1 expression cassette [SSU/mSPS/E9] are on two separate T-DNA's contained on a single binary vector. Using the 2-T vector was intended to produce marker-free soybean transformants. These were transformed into soybean as described in Example 29.

2. Plant Materials and Methods

R1 soybean including maize SPS positive and negative and wild-type control plants were grown in a standard growth chamber and in field of Jerseyville, Ill. In growth chamber plants were grown in 10-inch pots filled with Metro 350 with 14 hours light (700 μmol s⁻¹ m⁻²) at 30° C. and 10 hours dark at 24° C., 60% humidity. Plants were watered daily, and fertilized once a day with Peters 15-16-17 fertilizer (from Hummert International, Earth City, Mo.). Soybean seeds were planted in the field in Jerseyville, Ill. on Jun. 11, 2002. The presence of maize SPS gene in transformants was checked by PCR and Western blot as for transgenic corn plants (see prior examples).

In order to measure leaf sucrose and starch levels a fully expanded mature leaflet at top 4^{th} node of a plant was excise at R3 stage, frozen immediately in dry ice and later powdered in liquid N2 in Lab. Procedures of extraction and measurement of sucrose and starch were similar to the methods used for transgenic corn except gelatinization of soybean starch was done with 0.2 N KOH at 80 C followed by neutralization (see Fondy and Geiger, 1982) instead of boiling as was done in analyzing corn starch.

Leaf sucrose and starch both showed significant changes. Two events out of six showed significantly increased starch in the leaf, and all events showed increased starch in the leaf in a growth chamber study, and most showed increased starch in the leaf in a study in the field. All but one event showed increased sucrose in the leaf in a growth chamber study (significant events showed a range 16-24% when compared to negative segregants), and all showed increased leaf sucrose when planted in the field (significant events showing a 21-63% increase when compared to negative segregants). The heterologous expression of SPS causes advantageous effects in a soybean plant, although initial results using the Anabaena enzyme in soy did not result in plants expressing the gene.

### Example 29

Soybean was transformed using the following method. Dry A3244 soybean seeds were germinated by soaking in sterile distilled water (SDW) for three minutes, drained and allowed to slowly imbibe for 2 hours at which time Bean Germination Media (BGM) was added. At approximately 12 hours, seed axis explants were isolated by removing seed coats and cotyledons. Inoculation occurred 14 hours after the addition of SDW.

Explants were placed into sterile Plantcons with 20 mL of the plasmid being transformed and resuspended to an optical density A660 of approximately 0.3 in 1/10 Gamborg's B5 media (Gamborg et al., Exp. Cell Res., 50:151-158, 1968) containing 3% glucose, 1.68 mg/L BAP, 3.9 g/L MES, 0.2M acetosyringone, 1 mM galactronic acid, and 0.25 mg/L GA3. Each Plantcon was sonicated for 20 seconds in a L&R Quantrex S 140 sonicator that contained SDW+0.1% Triton X100 in the bath. Plantcons were held in place at approximately 2.5 cm below the surface of the bath liquid. Following sonication, explants were inoculated for an additional hour while shaking gently on an orbital shaker at ˜90 RPM. After inoculation, the Agrobacterium was removed. One sheet of square filter paper and 3 mL of co-culture media containing 0-500 mM lipoic acid were added. Co-culture media consisted of 1/10 Gamborg's B5 media containing 5% glucose, 1.68 mg/L BAP, 3.9 g/L, 0.2M acetosyringone, 1 mM galactronic acid and 0.25 mg/L GA3. Explants were incubated at 23° C., dark for 3 days.

Shoots were cut 5-8 weeks post-inoculation and rooted on Bean Rooting Media (BRM) containing 25 mM glyphosate and 100 mg/L Timetin.

BEAN GERMINATION MEDIA (BGM 2.5%)

| COMPOUND: | QUANTITY PER LITER | |
|---|---|---|
| BT STOCK #1 | 10 | mL |
| BT STOCK #2 | 10 | mL |
| BT STOCK #3 | 3 | mL |
| BT STOCK #4 | 3 | mL |
| BT STOCK #5 | 1 | mL |
| SUCROSE | 25 | g |
| Adjust to pH 5.8. | | |

DISPENSED IN 1 LITER MEDIA BOTTLES, AUTOCLAVED

| | | |
|---|---|---|
| ADDITIONS PRIOR TO USE: | PER 1 | L |
| CEFOTAXIME (50 mg/mL) | 2.5 | mL |
| FUNGICIDE STOCK | 3 | mL |

BT STOCK FOR BEAN GERMINATION MEDIUM (BGM)
Make and store each stock individually. Dissolve each chemical
thoroughly in the order listed before adding the next. Adjust
volume of each stock accordingly. Store at 4° C..

| | | |
|---|---|---|
| Bt Stock 1 (1 liter) | | |
| KNO3 | 50.5 | g |
| NH4NO3 | 24.0 | g |
| MgSO4*7H2O | 49.3 | g |
| KH2PO4 | 2.7 | g |
| Bt Stock 2 (1 liter) | | |
| CaCl2*2H2O | 17.6 | g |
| Bt Stock 3 (1 liter) | | |
| H3BO3 | 0.62 | g |
| MnSO4-H2O | 1.69 | g |
| ZnSO4-7H2O | 0.86 | g |
| KI | 0.083 | g |
| NaMoO4-2H2O | 0.072 | g |
| CuSO4-5H2O | 0.25 | mL of 1.0 mg/mL stock |
| CoC14-6H2O | 0.25 | mL of 1.0 mg/mL stock |
| Bt Stock 4 (1 liter) | | |
| Na2EDTA | 1.116 | g |
| FeSO47H2O | 0.834 | g |
| Bt Stock 5 (500 mL) | | |
| Store in a foil wrapped container | | |
| Thiamine-HC1 | 0.67 | g |
| Nicotinic Acid | 0.25 | g |
| Pyridoxine-HC1 | 0.41 | g |

FUNGICIDE STOCK (100 mL)

| | | |
|---|---|---|
| chlorothalonile (75% WP) | 1.0 | g |
| benomyl (50% WP) | 1.0 | g |
| captan (50% WP) | 1.0 | g |
| Add to 100 mL of sterile | | |
| distilled water. | | |
| Shake well before using. | | |
| Store 4° C. dark for no more | | |
| than one week. | | |

BEAN ROOTING MEDIA (BRM) (for 4 L)

| | | |
|---|---|---|
| MS Salts | 8.6 | g |
| Myo-Inositol (Cell Culture | .40 | g |
| Grade) | | |
| Soybean Rooting Media Vitamin | 8 | mL |
| Stock | | |
| L-Cysteine (10 mg/mL) | 40 | mL |
| Sucrose (Ultra Pure) | 120 | g |
| pH 5.8 | | |
| Washed Agar | 32 | g |

ADDITIONS AFTER AUTOCLAVING:

| | | |
|---|---|---|
| BRM Hormone Stock | 20.0 | mL |
| Ticarcillin/clavulanic acid | 4.0 | mL |
| (100 mg/mL Ticarcillin) | | |

VITAMIN STOCK FOR SOYBEAN ROOTING MEDIA (1 liter)

| | | |
|---|---|---|
| Glycine | 1.0 | g |
| Nicotinic Acid | 0.25 | g |
| Pyridoxine HCl | 0.25 | g |
| Thiamine HCl | 0.05 | g |

Dissolve one ingredient at a time, bring to volume, store in foil-covered
bottle in refrigerator for no more than one month.

1-20. (canceled)

21. A recombinant DNA molecule comprising:
(i) a mesophyll specific promoter, operably linked to
(ii) a DNA that encodes an sucrose phosphate synthase enzyme.

22-54. (canceled)

55. A recombinant DNA molecule of claim 21 wherein the promoter is a pyruvate orthophosphate dikinase promoter.

56. A recombinant DNA molecule of claim 21 wherein the promoter is a chlorophyll a/b binding protein promoter.

57. A recombinant DNA molecule of claim 21 wherein the sucrose phosphate synthase enzyme is from a plant.

58. A recombinant DNA molecule of claim 21 wherein the sucrose phosphate synthase enzyme is from an alga.

59. A recombinant DNA molecule of claim 21 wherein the sucrose phosphate synthase enzyme is from a cyanobacteria.

60. A seed comprising the recombinant DNA molecule of claim 21.

61. The seed of claim 60 wherein said seed is selected from the group consisting of corn and soybean.

62. The seed of claim 60 wherein said seed is selected from the group consisting of monocots and dicots.

63. A plant grown from the seed of claim 60.

64. A field of plants comprising plants of claim 63.

65. Plants of claim 64 wherein said plants are corn plants.

66. A method for expressing a sucrose phosphate synthase enzyme in a plant, comprising a comprising:
(a) transforming a plant with the DNA molecule of claim 1;
(b) obtaining transformed plant cells containing the nucleic acid sequence of step (a); and
(c) regenerating from the transformed plant cells a genetically transformed plant that express the heterologous sucrose phosphate synthase in the transformed plant wherein the transformed plant demonstrates elevated sucrose phosphate synthase production.

67. A method of increasing starch production in plant leaves comprising growing a plant with the recombinant DNA molecule or claim 21.

68. A method of increasing sugar production in plant leaves comprising growing a plant with the recombinant DNA molecule of claim 21.

69. A method of increasing yield in plants comprising growing a plant with the recombinant DNA molecule of claim 21.

70. The method of claim 69 wherein a field of plants is grown.

71. Crossing a plant comprising a recombinant DNA molecule of claim 21 with another plant.

72. Introgressing a recombinant DNA molecule for expressing a sucrose phosphate synthase into a plant line by crossing plants comprising the DNA molecule of claim 21 with other plants.

73. A corn plant comprising a recombinant DNA molecule of claim 21.
